(19) United States
(12) Patent Application Publication
(10) Pub. No.: US 2007/0275981 A1
(43) Pub. Date: Nov. 29, 2007

(54) (Hetero)Cyclycarboxamides for Controlling Pathogenic Fungi

(76) Inventors: Markus Gewehr, Kastellaun (DE); Thomas Grote, Wachenheim (DE); Bernd Muller, Frankenthal (DE); Wassilios Grammenos, Ludwigshafen (DE); Anja Schwogler, Mannheim (DE); Joachim Rheinheimer, Ludwigshafen (DE); Carsten Blettner, Hong Kong (CN); Peter Schafer, Ottersheim (DE); Frank Schieweck, Hessheim (DE); Oliver Wagner, Neustadt (DE); Jan Rether, Kaiserslautern (DE); Siefgried Strathmann, Limburgerhof (DE); Reinhard Stierl, Freinsheim (DE); Maria Scherer, Godramstein (DE)

Correspondence Address: BIRCH STEWART KOLASCH & BIRCH, PO BOX 747, FALLS CHURCH, VA, 22040-0747, US

(21) Appl. No.: 11/661,922

(22) PCT Filed: Sep. 5, 2005
(86) PCT No.: PCT/EP05/09534

§ 371 Date: Mar. 5, 2007

(30) Foreign Application Priority Data
Sep. 6, 2004 (DE) 10 2004 043 046.2

(51) Int. Cl. A01N 43/40; A01N 43/54; A01P 3/00; C07D 239/28; C07D 401/04

(52) U.S. Cl. 514/256; 514/275; 514/341; 544/297; 544/328; 546/275.4

(57) ABSTRACT

The invention relates to the use of (hetero)cyclylcarboxamides of general formula (I) and to the agriculturally useful salts thereof for controlling plant pathogenic fungi, the variables in formula (I) having the following designations: A represents phenyl or an at least monosaturated five-membered or six-membered heterocycle with 1, 2 or 3 heteroatoms as ring members selected from N, O, S, S(═O) and S(═O)₂, where phenyl and the at least monosaturated five-membered or six-membered heterocycle can be unsubstituted or substituted according to the description; Y represents oxygen or sulphur; R¹ represents H, OH, alkyl, cycloalkyl, alkoxy, halogenalkyl, halogencycloalkyl or halogenalkoxy; R² and R³ represent H, halogen, nitro, CN, alkyl, cycloalkyl, alkenyl, alkinyl, alkoxy, halogenalkyl, halogencycloalkyl, halogenalkenyl, halogenalkinyl or halogenalkoxy; R⁴ represents halogen, nitro, CN, alkyl, cycloalkyl, alkenyl, alkinyl, alkoxy, halogenalkyl, halogencycloalkyl, halogenalkenyl, halogenalkinyl or halogenalkoxy; R⁵ represents hydrogen, halogen, nitro, CN, OH, alkyl, cycloalkyl, alkenyl, alkinyl, alkoxy, alkenyloxy, alkinyloxy, alkoxyalkoxy, halogenalkyl, halogencycloalkyl, halogenalkenyl, halogenalkinyl or halogenalkoxy; alklthio, halogenalkylthio, alkylsulfinyl, halogenalkylsulfinyl, alkylsulfonyl, halogenalkylsulfonyl, —(CR⁶)═NOR⁷, —C(O)R⁸, NR⁹R¹⁰, —C(O)NR⁹R¹⁰, —C(S)NR⁹R¹⁰, phenyl or phenylalkyl, the phenyl ring in the last two radicals optionally comprising between 1 and 4 of the radicals cited for R⁴; R⁶, R⁷, R⁸, R⁹ and R¹⁰ have the designations cited in the description; the two radicals R⁴ and R⁵ bound to adjacent carbon atoms can also represent an alkylene chain with 3 to 5 members, wherein 1 or 2 non-adjacent CH₂ groups can also be replaced by O or S, and part or all of the hydrogens can be replaced by halogen; Ar represents phenyl, naphtyl or a five-membered or six-membered heteroaromatic radical with 1, 2 or 3 heteroatoms as ring members selected from N, O, and S, that can optionally also carry a fused benzene ring; and n represents 0, 1, 2, 3, or 4. The invention also relates to crop protection agents containing said compounds.

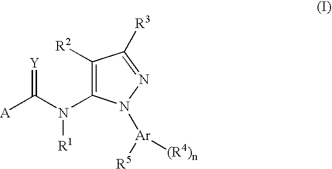

The present invention relates to a method for controlling harmful fungi, i.e. fungi which damage plants and which hereinbelow are also referred to as phytopathogenic fungi or plant-damaging fungi. Moreover, the invention relates to novel (hetero)cyclylcarboxamides and salts thereof, to crop protection compositions comprising them and to their use for controlling harmful fungi.

Fungicidally active heterocyclylcarboxamides of biphenylamines have been described in numerous publications, for example in EP-A 545099, EP-A 589301, EP-A 591699, WO 97/08148, WO 00/09482, WO 01/42223, WO 01/49665, JP 2001/302505, WO 02/059086, WO 02/064562, WO 03/066609, WO 03/069995 and WO 03/070705.

JP 08092223 describes fungicidally active heterocyclylcarboxanilides which carry a heteroaromatic radical on the phenyl ring.

Moreover, WO 00/09482 discloses trifluoromethylpyrrolecarboxamides of 3-aminothiophenes.

However, in particular at low application rates, the (heteroaryl)carboxanilides described in these publications are not entirely satisfactory with respect to their efficacy against phytopathogenic fungi.

WO 94/08999 describes herbicidally active 1-(1-H-pyrazol-3-yl)pyrazoles which may have an aminocarbonylphenyl radical in the 5-position.

WO 2005/023761 for its part describes benzamides of 1-phenylpyrazolamines which, by virtue of their action as cytokine inhibitors, are suitable for treating inflammatory disorders.

U.S. Pat. No. 6,548,512 and WO 98/28269 disclose amides of 1-phenyl-substituted pyrazoles which act as inhibitors of factor Xa.

An effect of the compounds described in the 4 last-mentioned publications on plant-damaging fungi is not mentioned.

Accordingly, it is an object of the present invention to provide fungicidally active compounds which overcome the disadvantages of the compounds known from the prior art and, in particular, have improved activity against phytopathogenic fungi at low application rates. Moreover, these compounds should be tolerated well by crop plants and, if possible, cause no or only little damage to useful animals.

We have found that this object is achieved by the (hetero)cyclylcarboxamides of the formula I described below and their agriculturally acceptable salts.

Accordingly, the present invention relates to a method for controlling phytopathogenic fungi which comprises treating the harmful fungi, their habitat or the plants, areas, materials or spaces to be kept free from them with a fungicidally effective amount of at least one (hetero)cyclylcarboxamide of the formula I defined below and/or at least one agriculturally acceptable salt thereof.

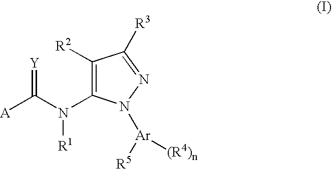

In formula I the variables n, A, Ar, Y, R¹, R², R³, R⁴ and R⁵ are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
   R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries 1, 2 or 3 radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R², R³ independently of one another are hydrogen, halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁴ is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁵ is hydrogen, halogen, nitro, CN, OH, C₁-C₆-alkyl, C₃-C₆-cycloalkyl, C₂-C₄-alkenyl, C₂-C₄-alkynyl, C₁-C₄-alkoxy, C₂-C₄-alkenyloxy, C₂-C₄-alkynyloxy, C₁-C₄-alkoxy-C₁-C₄-alkoxy, C₁-C₆-haloalkyl, C₃-C₆-halocycloalkyl, C₂-C₄-haloalkenyl, C₂-C₄-haloalkynyl or C₁-C₄-haloalkoxy; C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, —(CR⁶)═NOR⁷, —C(O)R⁸, NR⁹R¹⁰, —C(O)NR⁹R¹⁰, —C(S)NR⁹R¹⁰, phenyl or phenyl-C₁-C₄-alkyl, where the phenyl ring in the two last-mentioned radicals may optionally have 1, 2, 3 or 4 of the radicals mentioned under R⁴, where
   R⁶ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b};
   R⁷ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl and the phenyl group in phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl and phenyl-C₂-C₄-haloalkynyl may be unsubstituted or may carry one, two or three radicals R^{b};
   R⁸ is hydrogen, OH, C₁-C₄-alkyl, C₁-C₄-alkoxy, C₂-C₄-alkenyl, C₂-C₄-alkynyl, C₂-C₄-alkenyloxy, C₂-C₄-alkynyloxy, C₁-C₄-alkoxy-C₁-C₄-alkoxy, where some or all of the hydrogen atoms in the 7 last-mentioned groups may be replaced by halogen; and
   R⁹, R¹⁰ independently of one another are hydrogen, C₁-C₄-alkyl, C₂-C₄-alkenyl, C₂-C₄-alkynyl, where some or all of the hydrogen atoms in these groups may be replaced by halogen;
   and where two radicals R⁴ and R⁵ attached to adjacent carbon atoms may also be an alkylene chain having 3 to 5 members in which 1 or 2 non-adjacent CH₂ groups may also be replaced by oxygen or sulfur and in which some or all hydrogens may be replaced by halogen;
Ar is phenyl, naphthyl or a 5- or 6-membered heteroaromatic radical having 1, 2 or 3 heteroatoms selected from the group consisting of N, O and S as ring members, which radical may, if appropriate, also carry a fused-on benzene ring,
n is 0, 1, 2, 3 or 4;
   and their agriculturally useful salts.

Moreover, the present invention relates to the use of the (hetero)cyclylcarboxamides of the formula I and their agriculturally useful salts as fungicides, and to crop protection compositions comprising these compounds.

Some of the (hetero)cyclylcarboxamides of the formula I are known, for example from WO 94/08999, WO 2005/023761, U.S. Pat. No. 6,548,512 and WO 98/28269, or they are described in other publications as intermediates for preparing pharmaceutically active compounds. These are compounds of the formula I in which A is optionally substituted phenyl, and compounds of the formula I in which A is 1-phenylpyrazol-5-yl, 1-phenyl-3-methylpyrazol-5-yl, 1,3-dimethyl-4-chloropyrazol-5-yl, 5-nitropyrazol-2-yl, 1-ethyl-3-methyl-4-nitropyrazol-5-yl, 5-methyl-4-nitropyrazol-3-yl, 1-(4-chlorophenyl)-5-trifluoromethylpyrazol-4-yl, 2-thienyl, 2-furyl, 3-furyl, pyrazin-2-yl, 2,5-dihydropyrrol-2-yl, 2,3-dihydro-5-methyl-1,4-oxathian-6-yl or 5-methylisoxazol-3-yl, where Ar is phenyl.

Accordingly, the present invention also relates to (hetero)cyclylcarboxamides of the formula I and agriculturally useful salts thereof, where the variables n, Y, Ar, R¹, R², R³, R⁴ and R⁵ are as defined here and A is an at least monounsaturated 5- or 6-membered heterocycle having 1, 2 or 3 heteroatoms selected from the group consisting of N, O, S, S(═O) and S(═O)₂ as ring members, which heterocycle may be unsubstituted or may carry 1, 2 or 3 of the radicals R^{a} defined here, except for (hetero)cyclylcarboxamides of the formula I in which A is 1-phenylpyrazol-5-yl, 1-phenyl-3-methylpyrazol-5-yl, 1,3-dimethyl-4-chloropyrazol-5-yl, 5-nitropyrazol-3-yl, 1-ethyl-3-methyl-4-nitropyrazol-5-yl, 5-methyl-4-nitropyrazol-3-yl, 1-(4-chlorophenyl)-5-trifluoromethylpyrazol-4-yl, 2-thienyl, 3-methylthiophen-2-yl, 2-furyl, 3-furyl, pyrazin-2-yl, 2,5-dihydropyrrol-2-yl, 2,3-dihydro-5-methyl-1,4-thioxin-6-yl or 5-methylisoxazol-3-yl and Ar is phenyl.

The invention also provides (hetero)cyclylcarboxamides of the formula I′, 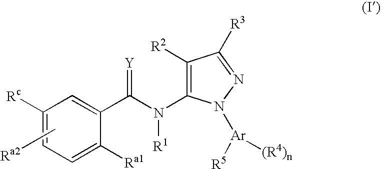
in which the variables Y, Ar, R¹, R², R³, R⁴ and R⁵ are as defined here, with the proviso that R⁵ is different from hydrogen, and in which
R^{a1} is C₁-C₄-alkyl, C₁-C₄-alkoxy, C₁-C₄-haloalkyl, C₁-C₄-haloalkoxy or halogen; and
R^{a2} is hydrogen, halogen, nitro, CN, C₁-C₄-alkyl, C₃-C₆-cycloalkyl, C₂-C₄-alkenyl, C₂-C₄-alkynyl or C₁-C₄-alkoxy, where the 5 last-mentioned groups may be substituted by halogen; and
R^{c} is selected from the group consisting of hydrogen, halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy
   and the agriculturally useful salts of I′.

Depending on the substitution pattern, the compounds of the formula I may contain one or more centers of chirality, in which case they are present as mixtures of enantiomers or diastereomers. The invention provides both the pure enantiomers or diastereomers and their mixtures. Suitable compounds of the formula I also include all possible stereoisomers (cis/trans isomers) and mixtures thereof.

Suitable agriculturally useful salts are especially the salts of those cations or the acid addition salts of those acids whose cations or anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ denotes in each case the possible number of carbon atoms in the respective substituent or substituent moiety. All carbon chains, i.e. all alkyl, haloalkyl, phenylalkyl, alkenyl, haloalkenyl, phenylalkenyl, alkynyl, haloalkynyl and phenylalkynyl moieties can be straight-chain or branched. Halogenated substituents preferably carry one to five identical or different halogen atoms. The term halogen denotes in each case fluorine, chlorine, bromine or iodine.

Examples of other meanings are:
C₁-C₄-alkyl and the alkyl moieties of C₁-C₄-alkylcarbonyl, C₁-C₄-alkoxycarbonyl, C₁-C₄-alkylsulfonyl, C₁-C₄-alkylthio, C₁-C₄-alkylaminocarbonyl, C₁-C₄-dialkylaminocarbonyl, etc.: CH₃, C₂H₅, CH₂—C₂H₅, CH(CH₃)₂, n-butyl, CH(CH₃)—C₂H₅, CH₂—CH(CH₃)₂ or C(CH₃)₃;
C₁-C₄-haloalkyl and the haloalkyl moieties of C₁-C₄-haloalkylcarbonyl, C₁-C₄-haloalkoxycarbonyl, C₁-C₄-haloalkylsulfonyl, C₁-C₄-haloalkylthio, etc.: a C₁-C₄-alkyl radical as mentioned above which is partially or fully substituted by fluorine, chlorine, bromine and/or iodine, i.e., for example, CH₂F, CHF₂, CF₃, CH₂Cl, CH(Cl)₂, C(Cl)₃, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, 1-(fluoro-methyl)-2-fluoroethyl, 1-(chloromethyl)-2-chloroethyl, 1-(bromo-methyl)-2-bromoethyl, 4-fluorobutyl, 4-chlorobutyl, 4-bromobutyl or nonafluorobutyl;
C₁-C₈-alkyl: a C₁-C₄-alkyl radical as mentioned above, or, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably CH₃, C₂H₅, CH₂—C₂H₅, CH(CH₃)₂, n-butyl, C(CH₃)₃, n-pentyl, n-hexyl, n-heptyl or n-octyl;
C₁-C₈-haloalkyl: a C₁-C₈-alkyl radical as mentioned above which is partially or fully substituted by fluorine, chlorine, bromine and/or iodine, i.e., for example, one of the radicals mentioned under C₁-C₄-haloalkyl or 5-fluoro-1-pentyl, 5-chloro-1-pentyl, 5-bromo-1-pentyl, 5-iodo-1-pentyl, 5,5,5-trichloro-1-pentyl, undeca-fluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;
C₂-C₄-alkenyl: unsaturated straight-chain or branched hydrocarbon radicals having 2 to 4 carbon atoms and a double bond in any position, for example ethenyl, 1-propenyl, 2-propenyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl;
C₂-C₆-alkenyl: C₂-C₄-alkenyl as mentioned above and also, for example: n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-1-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;
C₂-C₄-haloalkenyl: unsaturated straight-chain or branched hydrocarbon radicals having 2 to 4 carbon atoms and a double bond in any position (as mentioned above), where some or all of the hydrogen atoms in these groups are replaced by halogen atoms as mentioned above, in particular by fluorine, chlorine and bromine, i.e., for example, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl;
C₂-C₆-haloalkenyl: C₂-C₆-alkenyl as mentioned above which is partially or fully substituted by fluorine, chlorine, bromine and/or iodine, for example the radicals mentioned under C₂-C₄-haloalkenyl;
C₂-C₄-alkynyl: straight-chain or branched hydrocarbon groups having 2 to 4 carbon atoms and a triple bond in any position, for example ethynyl, 1-propynyl, 2-propynyl (=propargyl), 1-butynyl, 2-butynyl, 3-butynyl and 1-methyl-2-propynyl;
C₂-C₆-alkynyl: straight-chain or branched hydrocarbon groups having 2 to 6 carbon atoms and a triple bond in any position, for example ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl and 4-methylpent-2-yn-5-yl;
C₂-C₄-haloalkynyl: unsaturated straight-chain or branched hydrocarbon radicals having 2 to 4 carbon atoms and a triple bond in any position (as mentioned above), where some or all of the hydrogen atoms in these groups may be replaced by halogen atoms as mentioned above, in particular by fluorine, chlorine and bromine, i.e., for example, 1,1-difluoroprop-2-yn-1-yl, 4-fluorobut-2-yn-1-yl, 4-chlorobut-2-yn-1-yl or 1,1-difluorobut-2-yn-1-yl,
C₂-C₆-haloalkynyl: C₂-C₆-alkynyl as mentioned above which is partially or fully substituted by fluorine, chlorine, bromine and/or iodine, for example the radicals mentioned under C₂-C₄-haloalkynyl;
C₁-C₄-alkoxy: OCH₃, OC₂H₅, OCH₂—C₂H₅, OCH(CH₃)₂, n-butoxy, OCH(CH₃)—C₂H₅, OCH₂—CH(CH₃)₂ or OC(CH₃)₃;
C₁-C₄-haloalkoxy: a C₁-C₄-alkoxy radical as mentioned above which is partially or fully substituted by fluorine, chlorine, bromine and/or iodine, i.e., for example, OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoro-propoxy, 2,3-difluoropropoxy, 2-chloropropoxy, 3-chloropropoxy, 2,3-dichloro-propoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;
C₃-C₆-cycloalkyl: cyclopropyl, cyclobutyl, cyclopentyl or cyclohexyl;
C₃-C₆-cycloalkyl which is unsubstituted or mono- or polysubstituted by halogen: a C₃-C₆-cycloalkyl radical as mentioned above which is unsubstituted or partially or fully substituted by fluorine, chlorine, bromine and/or iodine, i.e., for example, 1-chlorocyclopropyl, 1-fluorocyclopropyl, 2-chlorocyclopropyl, 2-fluorocyclopropyl, 4-chlorocyclohexyl, 4-bromocyclohexyl;
phenyl-C₁-C₄-alkyl: C₁-C₄-alkyl which is substituted by phenyl, for example benzyl, 1- or 2-phenylethyl, 1-, 2- or 3-phenylpropyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₁-C₄-haloalkyl: C₁-C₄-haloalkyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-alkenyl: C₂-C₄-alkenyl which is substituted by phenyl, for example 1- or 2-phenylethenyl, 1-phenylprop-2-en-1-yl, 3-phenyl-1-propen-1-yl, 3-phenyl-2-propen-1-yl, 4-phenyl-1-buten-1-yl or 4-phenyl-2-buten-1-yl; where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-haloalkenyl: C₂-C₄-haloalkenyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-alkynyl: C₂-C₄-alkynyl, which is substituted by phenyl, for example 1-phenyl-2-propyn-1-yl, 3-phenyl-1-propyn-1-yl, 3-phenyl-2-propyn-1-yl, 4-phenyl-1-butyn-1-yl or 4-phenyl-2-butyn-1-yl; where the phenyl moiety of phenyl-C₂-C₄-alkynyl may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-haloalkynyl: C₂-C₄-haloalkynyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
an at least monounsaturated heterocycle having 5 or 6 ring members: a monocyclic heterocycle which has one, two or three ring members selected from the group consisting of O, S, S(═O), S(═O)₂ and N and which is at least monounsaturated or fully unsaturated, i.e. aromatic. Examples are aromatic 5-membered radicals, for example furyl, such as 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, 6-membered aromatic radicals, for example pyridynyl, such as 2-pyridynyl, 3-pyridynyl and 4-pyridynyl, pyridazynyl, such as 3-pyridazynyl and 4-pyridazynyl, pyrimidynyl, such as 2-pyrimidynyl, 4-pyrimidynyl and 5-pyrimidynyl, 2-pyrazynyl, 1,3,5-triazin-2-yl and 1,2,4-triazin-3-yl, partially unsaturated 5- or 6-membered heterocyclic radicals such as 1,2-dihydrofuran-2-yl, 1,2-dihydrofuran-3-yl, 1,2-dihydrothiophen-2-yl, 1,2-dihydrothiophen-3-yl, 1,3-dioxolanyl, 1,3-dithiolanyl, 2,3-dihydropyran-4-yl, 2,3-dihydropyran-5-yl, 2,3-dihydropyran-6-yl, 5,6-dihydro-4H-pyran-3-yl, 2,3-dihydrothiopyran-4-yl, 2,3-dihydrothiopyran-5-yl, 2,3-dihydrothiopyran-6-yl, 5,6-dihydro-4H-thiopyran-3-yl, 5,6-dihydro-[1,4]dioxin-2-yl, 5,6-dihydro-[1,4]dithiin-2-yl or 5,6-dihydro-[1,4]oxathiin-3-yl.

With a view to the fungicidal activity of the compounds I according to the invention, preference is given to those compounds of the formula I in which A is a cyclic radical A-1 to A-6: 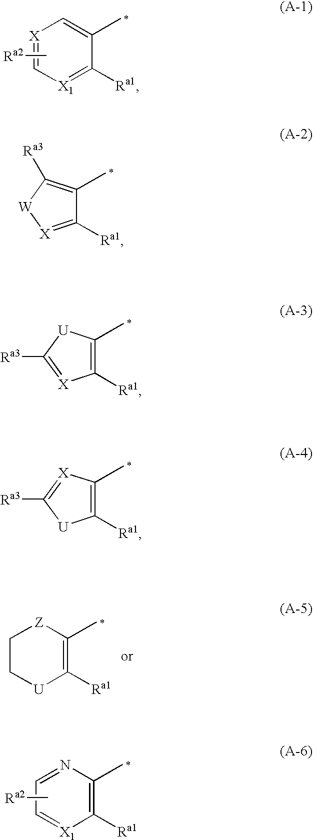
in which * denotes the point of attachment to C(═Y) and the variables are as defined below:
X, X₁ are each independently of one another N or CR^{c}, where R^{c} is H or has one of the meanings mentioned for R^{b}. In particular R^{c} is hydrogen;
W is S or N—R^{a4}, where R^{a4} is hydrogen, C₁-C₄-alkyl, C₁-C₄-haloalkyl, C₁-C₄-alkoxy, C₁-C₄-haloalkyl or phenyl which may be unsubstituted or may carry 1, 2 or 3 radicals R^{b}; R^{a4} in particular is hydrogen, C₁-C₄-alkyl or C₁-C₄-haloalkyl;
U is oxygen or sulfur;
Z is S, S(═O), S(═O)₂ or CH₂, particularly preferably S or CH₂;
R^{a1} is hydrogen, C₁-C₄-alkyl, C₁-C₄-alkoxy, C₁-C₄-haloalkyl, C₁-C₄-haloalkoxy or halogen, particularly preferably hydrogen, halogen, C₁-C₂-alkyl, C₁-C₂-alkoxy, C₁-C₂-fluoroalkoxy or C₁-C₂-fluoroalkyl;
R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, particularly preferably hydrogen, fluorine, chlorine or C₁-C₄-alkyl.

In the radicals of the formulae A-1, A-2, A-3, A-4, A-5 and A-6, the variables R^{a1}, R^{a2} and R^{a3} have in particular the following meanings:
R^{a1} is hydrogen, halogen, in particular fluorine or chlorine, C₁-C₄-alkyl or C₁-C₄-haloalkyl. In particular, R^{a1} is other than hydrogen. Particularly preferably, R^{a1} halogen, trifluoromethyl or methyl;
R^{a2} is hydrogen; and
R^{a3} is halogen, in particular fluorine or chlorine, or methyl.

In the formula A-2, W is preferably a group N—R^{a4}, where R^{a4} is as defined above and has in particular the meanings given as being preferred.

If X in the formulae A-1, A-2, A-3 or A-4 is a group C—R^{c}, R^{c} is preferably hydrogen.

In the formulae A-2, A-3 and A-4, X is in particular N. In the formula A-1, X is in particular CH.

In the formulae A-1 and A-6, X₁ is in particular N. In formula A-1, at least one of the groups X, X₂ is preferably N.

Examples for radicals A-1 are in particular: 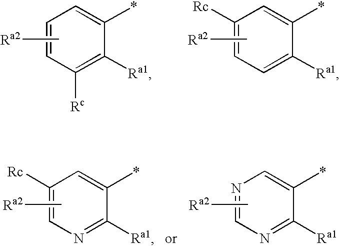
in which *, R^{a1}, R^{a2} and R^{c} have the meanings mentioned above, in particular the preferred meanings.

Examples for radicals A-2 are in particular: 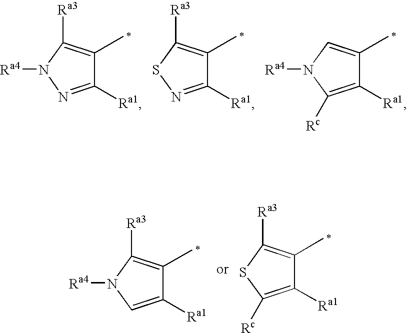
in which *, R^{a1}, R^{a3}, R^{a4} and R^{c} have the meanings mentioned above, in particular the preferred meanings.

Examples for radicals A-3 are in particular: 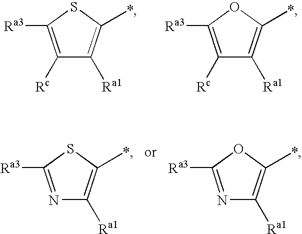
in which *, R^{a1}, R^{a3} and R^{c} have the meanings mentioned above, in particular the preferred meanings.

Examples for radicals A-4 are in particular: 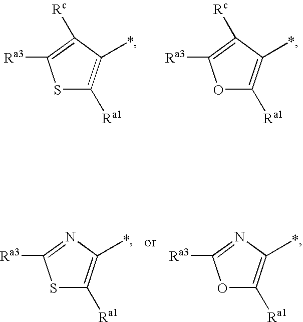
in which *, R^{a1}, R^{a3} and R^{c} have the meanings mentioned above, in particular the preferred meanings.

Examples for radicals A-5 are in particular: 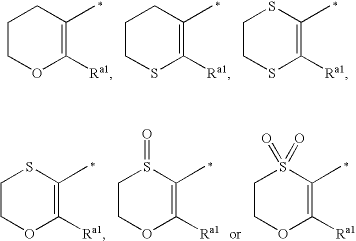
in which * and R^{a1} have the meanings mentioned above, in particular the preferred meanings.

Examples for radicals A-6 are in particular: 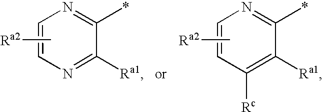
in which *, R^{a1}, R^{a2} and R^{c} have the meanings mentioned above, in particular the preferred meanings.

Examples for radicals A are: 2-chlorophenyl, 2-trifluoromethylphenyl, 2-difluoromethylphenyl, 2-methylphenyl, 2-chloropyridin-3-yl, 2-trifluoromethylpyridin-3-yl, 2-difluoromethylpyridin-3-yl, 2-methylpyridin-3-yl, 4-methylpyrimidin-5-yl, 4-trifluoromethylpyrimidin-5-yl, 4-difluoromethylpyrimidin-5-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloropyrazol-4-yl, 1-methyl-3-trifluoromethylpyrrol-4-yl, 1-methyl-3-difluoromethylpyrrol-4-yl, 2-methyl-4-trifluoromethylthiazol-5-yl, 2-methyl-4-difluoromethylthiazol-5-yl, 2,4-dimethylthiazol-5-yl, 2-methyl-5-trifluoromethylthiazol-4-yl, 2-methyl-5-difluoromethylthiazol-4-yl, 2,5-dimethylthiazol-4-yl, 2-methyl-4-trifluoromethyloxazol-5-yl, 2-methyl-4-difluoromethyloxazol-5-yl, 2,4-dimethyloxazol-5-yl, 2-trifluoromethylthiophen-3-yl, 5-methyl-2-trifluoromethylthiophen-3-yl, 2-methylthiophen-3-yl, 2,5-dimethylthiophen-3-yl, 3-trifluoromethylthiophen-2-yl, 3-methylthiophen-2-yl, 3,5-dimethylthiophen-2-yl, 5-methyl-3-trifluoromethyl-thiophen-2-yl, 2-trifluoromethylfuran-3-yl, 5-methyl-2-trifluoromethylfuran-3-yl, 2-methylfuran-3-yl, 2,5-dimethylfuran-3-yl, 2-methyl-5,6-dihydro[1,4]oxathiin-3-yl, 2-methyl-5,6-dihydro-4H-thiopyran-3-yl.

With particular preference, A is a radical A-1a, A-2a or A-3a, 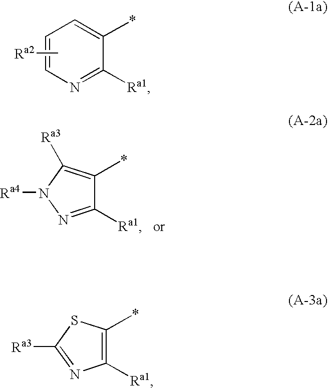
in which *, R^{a1}, R^{a2}, R^{a3} and R^{a4} have the meanings given above, in particular the preferred meanings.

Preference is given to radicals A-1a where R^{a1} is hydrogen, halogen, C₁-C₂-alkyl, C₁-C₂-alkoxy, C₁-C₂-fluoroalkoxy or C₁-C₂-fluoroalkyl; in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, and especially chlorine; where R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, especially hydrogen.

Preference is given to radicals A-2a where: R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, preferably hydrogen, halogen and C₁-C₄-alkyl, in particular halogen, hydrogen; and especially hydrogen; and R^{a4} is hydrogen, C₁-C₄-alkyl, C₁-C₄-haloalkyl, C₁-C₄-alkoxy, C₁-C₄-haloalkyl or phenyl, which may be unsubstituted or may carry 1, 2 or 3 radicals R^{b}, preferably hydrogen, C₁-C₄-alkyl or C₁-C₄-haloalkyl, especially methyl.

Preference is given to radicals A-3a where: R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, preferably hydrogen, halogen or C₁-C₄-alkyl, in particular hydrogen, methyl and especially methyl.

With particular preference, A is selected from the group consisting of:

A-1a where R^{a1}=halogen, especially chlorine and R^{a2}=hydrogen;

A-2a where R^{a1}=C₁-C₂-fluoroalkyl, especially trifluoromethyl, R^{a3}=hydrogen and R^{a4}=C₁-C₄-alkyl, especially methyl; and

A-3a where R^{a1}=C₁-C₂-fluoroalkyl, especially trifluoromethyl, and R^{a3}=C₁-C₄-alkyl, especially methyl.

With a view to their fungicidal activity, preference is given to (hetero)cyclylcarboxamides of the formula I in which the variables Y, R¹, R², R³, R⁴, R⁵ and n independently of one another and preferably in combination have the following meanings:
Y is O;
R¹ is hydrogen, OH, C₁-C₄-alkyl, in particular H, OH or methyl and particularly preferably hydrogen;
R² is hydrogen, C₁-C₄-alkyl, C₁-C₄-haloalkyl, nitro, cyano or halogen; particularly preferably hydrogen, methyl, ethyl, CF₃, nitro, cyano or halogen;
R³ is hydrogen, C₁-C₄-alkyl, C₁-C₄-haloalkyl, nitro, cyano or halogen; particularly preferably hydrogen, methyl, ethyl, CF₃, nitro, cyano or halogen;
R⁴ is C₁-C₄-alkyl, C₁-C₄-alkoxy, C₁-C₄-haloalkyl, C₁-C₄-haloalkoxy, nitro, cyano or halogen; particularly preferably C₁-C₄-alkyl, C₁-C₄-alkoxy, nitro, cyano or halogen and especially methyl, methoxy, fluorine, chlorine, bromine, nitro or cyano;
n is 0 or 1, particularly preferably 0;
R⁵ is hydrogen, halogen, CN, NO₂, NH₂, C(O)NH₂, C(S)NH₂, C₁-C₆-alkyl, C₂-C₆-alkenyl, C₁-C₆-haloalkyl, C₁-C₄-alkoxy, C₁-C₄-haloalkoxy, C₃-C₆-cycloalkyl, C₃-C₆-halocycloalkyl, C₁-C₄-alkylcarbonyl, C₁-C₄-alkoxycarbonyl, C₁-C₄-alkylamino, di-C₁-C₄-alkylamino, C₁-C₄-alkylthio, C₁-C₄-alkylsulfonyl, C₁-C₄-alkylaminocarbonyl, C₁-C₄-dialkylaminocarbonyl, phenyl, phenyl-C₁-C₄-alkyl, where phenyl in the two last-mentioned radicals may be unsubstituted or may carry one, two or three radicals R^{b}, or a group —C(R⁶)═NOR⁷, or R⁴ with R⁵ may also be methylenedioxy, dichloromethylenedioxy or difluoromethylenedioxy,
   in particular hydrogen, halogen, C₁-C₄-alkyl, C₁-C₄-haloalkyl, phenyl which may be unsubstituted or may carry one, two or three radicals R^{b}, or a group —C(R⁶)═NOR⁷;
Ar is phenyl, a six-membered heteroaromatic having 1 or 2 nitrogen atoms, such as 2-, 3- or 4-pyridinyl, 2-, 4- or 5-pyrimidinyl, 2- or 3-pyrazinyl or 3- or 4-pyridazinyl or a five-membered heteroaromatic having one nitrogen atom and optionally a further heteroatom selected from the group consisting of O, S and N, such as 3-, 4- or 5-pyrazolyl, 2-, 4- or 5-imidazolyl, 2-, 4- or 5-oxazolyl, 3-, 4- or 5-isoxazolyl, 2-, 4- or 5-thiazolyl, 3-, 4- or 5-isothiazolyl, 2- or 3-furyl, 2- or 3-thienyl, or 2- or 3-pyrrolyl. Ar is in particular phenyl, 2-, 3- or 4-pyridinyl, 2-, 4- or 5-pyrimidinyl and especially phenyl.

Furthermore, R^{b}, R⁶, R⁷, R⁸, R⁹ and R¹⁰ preferably have the following meanings:
R^{b} is halogen, nitro, CN, C₁-C₄-alkyl, especially methyl, C₁-C₄-alkoxy, especially methoxy, C₁-C₄-haloalkyl, especially trifluoromethyl, or C₁-C₄-haloalkoxy, especially difluoromethoxy,
R⁶ is hydrogen, C₁-C₆-alkyl, C₁-C₆-haloalkyl, C₃-C₆-cycloalkyl, C₃-C₆-halocycloalkyl, phenyl, phenyl-C₁-C₄-alkyl, phenyl-C₁-C₄-haloalkyl, where phenyl in the three last-mentioned radicals may be unsubstituted or may carry one, two or three radicals R^{b}; preferably hydrogen, C₁-C₄-alkyl, C₁-C₄-haloalkyl, phenyl which may be unsubstituted or may carry one, two or three radicals R^{b}; in particular, R⁶ is hydrogen or C₁-C₄-alkyl;
R⁷ is C₁-C₆-alkyl, C₁-C₆-haloalkyl, C₃-C₆-cycloalkyl, C₃-C₆-halocycloalkyl, C₃-C₆-alkenyl, C₃-C₆-haloalkenyl, C₃-C₄-alkynyl, C₃-C₄-haloalkynyl, phenyl-C₁-C₂-alkyl or phenyl, where phenyl in the two last-mentioned radicals may be unsubstituted or may carry one or two halogen groups, especially fluorine or chlorine,
   in particular C₁-C₄-alkyl, C₃-C₄-alkynyl, phenyl or benzyl;
R⁸ is hydrogen, OH, C₁-C₄-alkyl or C₁-C₄-alkoxy;
R⁹, R¹⁰ independently of one another are H or C₁-C₄-alkyl.

Particular preference is furthermore given to the (heterocyclyl)carboxamides of the formula I, in which Ar, R¹, R², R³, R⁴, R⁵ and n have the meanings mentioned above and in particular the preferred meanings, Y is oxygen and A is selected from the group consisting of:

A-1, where X and X₁ are each nitrogen, R^{a1} has the meanings mentioned above, in particular the preferred meanings, and is especially methyl, trifluoromethyl, chlorine, bromine or fluorine; R^{a2} has the meanings mentioned above and is especially hydrogen;

A-2, where X is N, W is S, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen;

A-2, where X is CH, W is N—R^{a4}, where R^{a4} is C₁-C₄-alkyl, especially methyl, R^{a1} has the meanings mentioned above, in particular the preferred meanings and is especially methyl, fluorine, chlorine, bromine or trifluoromethyl; R^{a3} has the meanings mentioned above, in particular the preferred meanings, and is especially hydrogen;

A-3, where U is O, X is N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-3, where U is S, X is CH, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-4, where U is O, X is CH or N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-4, where U is S, X is CH or N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-5, where U is oxygen, Z is CH₂, S, S(═O) or S(═O)₂ and R^{a1} has the meanings mentioned above, in particular the preferred meanings, and is especially methyl, fluorine, chlorine, bromine or trifluoromethyl;

A-6, where X₁ is nitrogen, R^{a2} has the meanings mentioned above and is especially hydrogen; R^{a1} has the meanings mentioned above, in particular the preferred meanings, and is especially methyl, fluorine, chlorine, bromine or trifluoromethyl.

In particular with a view to their use as fungicides, preference is given to the compounds of the formulae I-A and I-B, 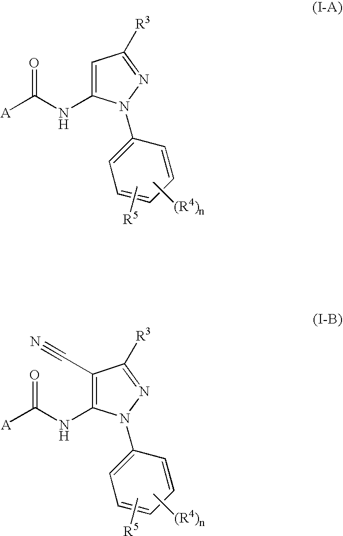
in which A, R³, R⁴, R⁵ and n are as defined above, and from among these in particular those in which:
A is a radical A-1, A-2, A-3, A-4, A-5 or A-6, in particular A-1a, A-2a or A-3a, and especially a radical selected from the group consisting of 2-chloropyridin-3-yl, 2-trifluoromethylpyridin-3-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloro-pyrazol-4-yl, 1-methyl-3-trifluoromethylpyrrol-4-yl, 2-methyl-4-trifluoromethylthiazol-5-yl, 2-methyl-4-difluoromethylthiazol-5-yl, 2,4-dimethylthiazol-5-yl, 2-methyl-5-trifluoromethylthiazol-4-yl, 2,5-dimethyl-thiazol-4-yl, 2-methyl-4-trifluoromethyloxazol-5-yl, 2-trifluoromethylthiophen-3-yl, 5-methyl-2-trifluoromethylthiophen-3-yl, 3-trifluoromethylthiophen-2-yl and 2,5-dimethylfuran-3-yl;
R³ is selected from the group consisting of H, methyl, trifluoromethyl, CN, NO₂ and halogen, especially H and trifluoromethyl;
(R⁴)ₙ is either not present (i.e. n=0) or is fluorine, chlorine, bromine, methyl, methoxy, dimethoxy, bromine+chlorine or dichloro; and
R⁵ is hydrogen, halogen, CN, NO₂, NH₂, C(O)NH₂, C(S)NH₂, C₁-C₆-alkyl, C₂-C₆-alkenyl, C₁-C₆-haloalkyl, C₁-C₄-alkoxy, C₁-C₄-haloalkoxy, C₃-C₆-cycloalkyl, C₃-C₆-halocycloalkyl, C₁-C₄-alkylcarbonyl, C₁-C₄-alkoxycarbonyl, C₁-C₄-alkylamino, di-C₁-C₄-alkylamino, C₁-C₄-alkylthio, C₁-C₄-alkylsulfonyl, C₁-C₄-alkylaminocarbonyl, C₁-C₄-dialkylaminocarbonyl, phenyl, phenyl-C₁-C₄-alkyl, where phenyl in the two last-mentioned radicals may be unsubstituted or may carry one, two or three radicals R^{b}, or a group —C(R⁶)═NOR⁷ or R⁴ with R⁵ may also be methylenedioxy, dichloromethylenedioxy or difluoromethylenedioxy,
   in particular hydrogen, halogen, C₁-C₄-alkyl, C₁-C₄-haloalkyl, phenyl which may be unsubstituted or may carry one, two or three radicals R^{b}, or a group —C(R⁶)═NOR⁷.

Examples of these are the individual compounds of the formulae I-A and I-B compiled in tables 1 to 19 below, where the variables R³, R⁵ and (R⁴)ₙ each have the meanings given in one row of table A and the variable A has the meaning given in the respective table. In the case of compounds containing double bonds this comprises both the isomerically pure E isomers, Z isomers and isomer mixtures.

TABLE A

| No. | R³ | R⁵ | (R⁴)ₙ |
|---|---|---|---|
| 1. | H | H | — |
| 2. | H | 2-F | — |
| 3. | H | 3-F | — |
| 4. | H | 4-F | — |
| 5. | H | 2-F | 3-F |
| 6. | H | 2-F | 4-F |
| 7. | H | 2-F | 5-F |
| 8. | H | 2-F | 6-F |
| 9. | H | 3-F | 4-F |
| 10. | H | 3-F | 5-F |
| 11. | H | 2-Cl | — |
| 12. | H | 3-Cl | — |
| 13. | H | 4-Cl | — |
| 14. | H | 2-Cl | 3-Cl |
| 15. | H | 2-Cl | 4-Cl |
| 16. | H | 2-Cl | 5-Cl |
| 17. | H | 2-Cl | 6-Cl |
| 18. | H | 3-Cl | 4-Cl |
| 19. | H | 3-Cl | 5-Cl |
| 20. | H | 2-Cl | 3,4-Cl₂ |
| 21. | H | 2-Cl | 3,5-Cl₂ |
| 22. | H | 2-Cl | 3,6-Cl₂ |
| 23. | H | 2-Cl | 4,5-Cl₂ |
| 24. | H | 2-Cl | 4,6-Cl₂ |
| 25. | H | 3-Cl | 4,5-Cl₂ |
| 26. | H | 2-Br | — |
| 27. | H | 3-Br | — |
| 28. | H | 4-Br | — |
| 29. | H | 2-Br | 3-Br |
| 30. | H | 2-Br | 4-Br |
| 31. | H | 2-Br | 5-Br |
| 32. | H | 2-Br | 6-Br |
| 33. | H | 3-Br | 4-Br |
| 34. | H | 3-Br | 5-Br |
| 35. | H | 2-F | 3-Cl |
| 36. | H | 2-F | 4-Cl |
| 37. | H | 2-F | 5-Cl |
| 38. | H | 2-F | 3-Br |
| 39. | H | 2-F | 4-Br |
| 40. | H | 2-F | 5-Br |
| 41. | H | 2-Cl | 3-Br |
| 42. | H | 2-Cl | 4-Br |
| 43. | H | 2-Cl | 5-Br |
| 44. | H | 3-F | 4-Cl |
| 45. | H | 3-F | 5-Cl |
| 46. | H | 3-F | 6-Cl |
| 47. | H | 3-F | 4-Br |
| 48. | H | 3-F | 5-Br |
| 49. | H | 3-F | 6-Br |
| 50. | H | 3-Cl | 4-Br |
| 51. | H | 3-Cl | 5-Br |
| 52. | H | 3-Cl | 6-Br |
| 53. | H | 4-F | 5-Cl |
| 54. | H | 4-F | 6-Cl |
| 55. | H | 4-F | 5-Br |
| 56. | H | 4-F | 6-Br |
| 57. | H | 4-Cl | 5-Br |
| 58. | H | 5-F | 6-Cl |
| 59. | H | 5-F | 6-Br |
| 60. | H | 5-Cl | 6-Br |
| 61. | H | 3-Br | 4-Cl, 5-Br |
| 62. | H | 2-CN | — |
| 63. | H | 3-CN | — |
| 64. | H | 4-CN | — |
| 65. | H | 2-NO₂ | — |
| 66. | H | 3-NO₂ | — |
| 67. | H | 4-NO₂ | — |
| 68. | H | 2-CH₃ | — |
| 69. | H | 3-CH₃ | — |
| 70. | H | 4-CH₃ | — |
| 71. | H | 2-CH₃ | 3-CH₃ |
| 72. | H | 2-CH₃ | 4-CH₃ |
| 73. | H | 2-CH₃ | 5-CH₃ |
| 74. | H | 2-CH₃ | 6-CH₃ |
| 75. | H | 3-CH₃ | 4-CH₃ |
| 76. | H | 3-CH₃ | 5-CH₃ |
| 77. | H | 2-C₂H₅ | — |
| 78. | H | 3-C₂H₅ | — |
| 79. | H | 4-C₂H₅ | — |
| 80. | H | 2-i-C₃H₇ | — |
| 81. | H | 3-i-C₃H₇ | — |
| 82. | H | 4-i-C₃H₇ | — |
| 83. | H | 3-tert-C₄H₉ | — |
| 84. | H | 4-tert-C₄H₉ | — |
| 85. | H | 2-vinyl | — |
| 86. | H | 3-vinyl | — |
| 87. | H | 4-vinyl | — |
| 88. | H | 2-allyl | — |
| 89. | H | 3-allyl | — |
| 90. | H | 4-allyl | — |
| 91. | H | 2-C₆H₅ | — |
| 92. | H | 3-C₆H₅ | — |
| 93. | H | 4-C₆H₅ | — |
| 94. | H | 5-tert-C₄H₉ | 3-CH₃ |
| 95. | H | 2-OH | — |
| 96. | H | 3-OH | — |
| 97. | H | 4-OH | — |
| 98. | H | 2-OCH₃ | — |
| 99. | H | 3-OCH₃ | — |
| 100. | H | 4-OCH₃ | — |
| 101. | H | 2-OCH₃ | 3-OCH₃ |
| 102. | H | 2-OCH₃ | 4-OCH₃ |
| 103. | H | 2-OCH₃ | 5-OCH₃ |
| 104. | H | 3-OCH₃ | 4-OCH₃ |
| 105. | H | 3-OCH₃ | 5-OCH₃ |
| 106. | H | 3-OCH₃ | 4,5-(OCH₃)₂ |
| 107. | H | 2-OC₂H₅ | — |
| 108. | H | 3-OC₂H₅ | — |
| 109. | H | 4-OC₂H₅ | — |
| 110. | H | 2-O-(n-C₃H₇) | — |
| 111. | H | 3-O-(n-C₃H₇) | — |
| 112. | H | 4-O-(n-C₃H₇) | — |
| 113. | H | 2-O-(i-C₃H₇) | — |
| 114. | H | 3-O-(i-C₃H₇) | — |
| 115. | H | 4-O-(i-C₃H₇) | — |
| 116. | H | 4-O-(n-C₄H₉) | — |
| 117. | H | 3-O-(t-C₄H₉) | — |
| 118. | H | 4-O-(t-C₄H₉) | — |
| 119. | H | 2-O-allyl | — |
| 120. | H | 3-O-allyl | — |
| 121. | H | 4-O-allyl | — |
| 122. | H | 2-CF₃ | — |
| 123. | H | 3-CF₃ | — |
| 124. | H | 4-CF₃ | — |
| 125. | H | 2-acetyl | — |
| 126. | H | 3-acetyl | — |
| 127. | H | 4-acetyl | — |
| 128. | H | 2-methoxycarbonyl | — |
| 129. | H | 3-methoxycarbonyl | — |
| 130. | H | 4-methoxycarbonyl | — |
| 131. | H | 2-aminocarbonyl | — |
| 132. | H | 3-aminocarbonyl | — |
| 133. | H | 4-aminocarbonyl | — |
| 134. | H | 2-dimethylaminocarbonyl | — |
| 135. | H | 3-dimethylaminocarbonyl | — |
| 136. | H | 4-dimethylaminocarbonyl | — |
| 137. | H | 2-(N-methylaminocarbonyl) | — |
| 138. | H | 3-(N-methylaminocarbonyl) | — |
| 139. | H | 4-(N-methylaminocarbonyl) | — |
| 140. | H | 2-H₂N | — |
| 141. | H | 3-H₂N | — |
| 142. | H | 4-H₂N | — |
| 143. | H | 2-aminothiocarbonyl | — |
| 144. | H | 3-aminothiocarbonyl | — |
| 145. | H | 4-aminothiocarbonyl | — |

| | | |
|---|---|---|
| 146. | H | 3,4-methylenedioxy |
| 147. | H | 3,4-difluoromethylenedioxy |
| 148. | H | 2,3-methylenedioxy |

| | | | |
|---|---|---|---|
| 149. | H | 2-SCH₃ | — |
| 150. | H | 3-SCH₃ | — |
| 151. | H | 4-SCH₃ | — |
| 152. | H | 2-SO₂CH₃ | — |
| 153. | H | 3-SO₂CH₃ | — |
| 154. | H | 4-SO₂CH₃ | — |
| 155. | H | 2-OCF₃ | — |
| 156. | H | 3-OCF₃ | — |
| 157. | H | 4-OCF₃ | — |
| 158. | H | 2-OCHF₂ | — |
| 159. | H | 3-OCHF₂ | — |
| 160. | H | 4-OCHF₂ | — |
| 161. | H | 3-CF₃ | 4-OCF₃ |
| 162. | H | 2-NHCH₃ | — |
| 163. | H | 3-NHCH₃ | — |
| 164. | H | 4-NHCH₃ | — |
| 165. | H | 2-N(CH₃)₂ | — |
| 166. | H | 3-N(CH₃)₂ | — |
| 167. | H | 4-N(CH₃)₂ | — |
| 168. | H | 2-ethoxycarbonyl | — |
| 169. | H | 3-ethoxycarbonyl | — |
| 170. | H | 4-ethoxycarbonyl | — |
| 171. | H | 2-CH₂CH₂F | — |
| 172. | H | 3-CH₂CH₂F | — |
| 173. | H | 4-CH₂CH₂F | — |
| 174. | H | 2-CH₂CF₃ | — |
| 175. | H | 3-CH₂CF₃ | — |
| 176. | H | 4-CH₂CF₃ | — |
| 177. | H | 2-CF₂CHF₂ | — |
| 178. | H | 3-CF₂CHF₂ | — |
| 179. | H | 4-CF₂CHF₂ | — |
| 180. | H | 2-CHF₂ | — |
| 181. | H | 3-CHF₂ | — |
| 182. | H | 4-CHF₂ | — |
| 183. | H | 2-(1′-oxo-n-prop-1-yl) | — |
| 184. | H | 3-(1′-oxo-n-prop-1-yl) | — |
| 185. | H | 4-(1′-oxo-n-prop-1-yl) | — |
| 186. | H | 2-(1′-oxoisoprop-1-yl) | — |
| 187. | H | 3-(1′-oxoisoprop-1-yl) | — |
| 188. | H | 4-(1′-oxoisoprop-1-yl) | — |
| 189. | H | 3-cyclopropyl | — |
| 190. | H | 4-cyclopropyl | — |
| 191. | H | 4-cyclohexyl | — |
| 192. | H | 2-methoxyiminomethyl | — |
| 193. | H | 3-methoxyiminomethyl | — |
| 194. | H | 4-methoxyiminomethyl | — |
| 195. | H | 2-ethoxyiminomethyl | — |
| 196. | H | 3-ethoxyiminomethyl | — |
| 197. | H | 4-ethoxyiminomethyl | — |
| 198. | H | 2-isopropyloxyiminomethyl | — |
| 199. | H | 3-isopropyloxyiminomethyl | — |
| 200. | H | 4-isopropyloxyiminomethyl | — |
| 201. | H | 2-allyloxyiminomethyl | — |
| 202. | H | 3-allyloxyiminomethyl | — |
| 203. | H | 4-allyloxyiminomethyl | — |
| 204. | H | 2-phenoxyiminomethyl | — |
| 205. | H | 3-phenoxyiminomethyl | — |
| 206. | H | 4-phenoxyiminomethyl | — |
| 207. | H | 2-benzyloxyiminomethyl | — |
| 208. | H | 3-benzyloxyiminomethyl | — |
| 209. | H | 4-benzyloxyiminomethyl | — |
| 210. | H | 2-(1-methoxyiminoeth-1-yl) | — |
| 211. | H | 3-(1-methoxyiminoeth-1-yl) | — |
| 212. | H | 4-(1-methoxyiminoeth-1-yl) | — |
| 213. | H | 2-(1-isopropyloxyiminoeth-1-yl) | — |
| 214. | H | 3-(1-isopropyloxyiminoeth-1-yl) | — |
| 215. | H | 4-(1-isopropyloxyiminoeth-1-yl) | — |
| 216. | H | 2-(1-allyloxyiminoeth-1-yl) | — |
| 217. | H | 3-(1-allyloxyiminoeth-1-yl) | — |
| 218. | H | 4-(1-allyloxyiminoeth-1-yl) | — |
| 219. | H | 2-(1-phenoxyiminoeth-1-yl) | — |
| 220. | H | 3-(1-phenoxyiminoeth-1-yl) | — |
| 221. | H | 4-(1-phenoxyiminoeth-1-yl) | — |
| 222. | H | 2-(1-benzyloxyiminoeth-1-yl) | — |
| 223. | H | 3-(1-benzyloxyiminoeth-1-yl) | — |
| 224. | H | 4-(1-benzyloxyiminoeth-1-yl) | — |
| 225. | H | 2-(1-ethoxyimino-n-prop-1-yl)- | — |
| 226. | H | 3-(1-ethoxyimino-n-prop-1-yl)- | — |
| 227. | H | 4-(1-ethoxyimino-n-prop-1-yl) | — |
| 228. | H | 2-(1-allyloxyimino-n-prop-1-yl) | — |
| 229. | H | 3-(1-allyloxyimino-n-prop-1-yl) | — |
| 230. | H | 4-(1-allyloxyimino-n-prop-1-yl) | — |
| 231. | CH₃ | H | — |
| 232. | CH₃ | 2-F | — |
| 233. | CH₃ | 3-F | — |
| 234. | CH₃ | 4-F | — |
| 235. | CH₃ | 2-F | 3-F |
| 236. | CH₃ | 2-F | 4-F |
| 237. | CH₃ | 2-F | 5-F |
| 238. | CH₃ | 2-F | 6-F |
| 239. | CH₃ | 3-F | 4-F |
| 240. | CH₃ | 3-F | 5-F |
| 241. | CH₃ | 2-Cl | — |
| 242. | CH₃ | 3-Cl | — |
| 243. | CH₃ | 4-Cl | — |
| 244. | CH₃ | 2-Cl | 3-Cl |
| 245. | CH₃ | 2-Cl | 4-Cl |
| 246. | CH₃ | 2-Cl | 5-Cl |
| 247. | CH₃ | 2-Cl | 6-Cl |
| 248. | CH₃ | 3-Cl | 4-Cl |
| 249. | CH₃ | 3-Cl | 5-Cl |
| 250. | CH₃ | 2-Cl | 3,4-Cl₂ |
| 251. | CH₃ | 2-Cl | 3,5-Cl₂ |
| 252. | CH₃ | 2-Cl | 3,6-Cl₂ |
| 253. | CH₃ | 2-Cl | 4,5-Cl₂ |
| 254. | CH₃ | 2-Cl | 4,6-Cl₂ |
| 255. | CH₃ | 3-Cl | 4,5-Cl₂ |
| 256. | CH₃ | 2-Br | — |
| 257. | CH₃ | 3-Br | — |
| 258. | CH₃ | 4-Br | — |
| 259. | CH₃ | 2-Br | 3-Br |
| 260. | CH₃ | 2-Br | 4-Br |
| 261. | CH₃ | 2-Br | 5-Br |
| 262. | CH₃ | 2-Br | 6-Br |
| 263. | CH₃ | 3-Br | 4-Br |
| 264. | CH₃ | 3-Br | 5-Br |
| 265. | CH₃ | 2-F | 3-Cl |
| 266. | CH₃ | 2-F | 4-Cl |
| 267. | CH₃ | 2-F | 5-Cl |
| 268. | CH₃ | 2-F | 3-Br |
| 269. | CH₃ | 2-F | 4-Br |
| 270. | CH₃ | 2-F | 5-Br |
| 271. | CH₃ | 2-Cl | 3-Br |
| 272. | CH₃ | 2-Cl | 4-Br |
| 273. | CH₃ | 2-Cl | 5-Br |
| 274. | CH₃ | 3-F | 4-Cl |
| 275. | CH₃ | 3-F | 5-Cl |
| 276. | CH₃ | 3-F | 6-Cl |
| 277. | CH₃ | 3-F | 4-Br |
| 278. | CH₃ | 3-F | 5-Br |
| 279. | CH₃ | 3-F | 6-Br |
| 280. | CH₃ | 3-Cl | 4-Br |
| 281. | CH₃ | 3-Cl | 5-Br |
| 282. | CH₃ | 3-Cl | 6-Br |
| 283. | CH₃ | 4-F | 5-Cl |
| 284. | CH₃ | 4-F | 6-Cl |
| 285. | CH₃ | 4-F | 5-Br |
| 286. | CH₃ | 4-F | 6-Br |
| 287. | CH₃ | 4-Cl | 5-Br |
| 288. | CH₃ | 5-F | 6-Cl |
| 289. | CH₃ | 5-Fr | 6-Br |
| 290. | CH₃ | 5-Cl | 6-Br |
| 291. | CH₃ | 3-Br | 4-Cl, 5-Br |
| 292. | CH₃ | 2-CN | — |
| 293. | CH₃ | 3-CN | — |
| 294. | CH₃ | 4-CN | — |
| 295. | CH₃ | 2-NO₂ | — |
| 296. | CH₃ | 3-NO₂ | — |
| 297. | CH₃ | 4-NO₂ | — |
| 298. | CH₃ | 2-CH₃ | — |
| 299. | CH₃ | 3-CH₃ | — |
| 300. | CH₃ | 4-CH₃ | — |
| 301. | CH₃ | 2-CH₃ | 3-CH₃ |
| 302. | CH₃ | 2-CH₃ | 4-CH₃ |
| 303. | CH₃ | 2-CH₃ | 5-CH₃ |
| 304. | CH₃ | 2-CH₃ | 6-CH₃ |
| 305. | CH₃ | 3-CH₃ | 4-CH₃ |
| 306. | CH₃ | 3-CH₃ | 5-CH₃ |
| 307. | CH₃ | 2-C₂H₅ | — |
| 308. | CH₃ | 3-C₂H₅ | — |
| 309. | CH₃ | 4-C₂H₅ | — |
| 310. | CH₃ | 2-i-C₃H₇ | — |
| 311. | CH₃ | 3-i-C₃H₇ | — |
| 312. | CH₃ | 4-i-C₃H₇ | — |
| 313. | CH₃ | 3-tert-C₄H₉ | — |
| 314. | CH₃ | 4-tert-C₄H₉ | — |
| 315. | CH₃ | 2-vinyl | — |
| 316. | CH₃ | 3-vinyl | — |
| 317. | CH₃ | 4-vinyl | — |
| 318. | CH₃ | 2-allyl | — |
| 319. | CH₃ | 3-allyl | — |
| 320. | CH₃ | 4-allyl | — |
| 321. | CH₃ | 2-C₆H₅ | — |
| 322. | CH₃ | 3-C₆H₅ | — |
| 323. | CH₃ | 4-C₆H₅ | — |
| 324. | CH₃ | 3-CH₃ | 5-tert-C₄H₉ |
| 325. | CH₃ | 2-OH | — |
| 326. | CH₃ | 3-OH | — |
| 327. | CH₃ | 4-OH | — |
| 328. | CH₃ | 2-OCH₃ | — |
| 329. | CH₃ | 3-OCH₃ | — |
| 330. | CH₃ | 4-OCH₃ | — |
| 331. | CH₃ | 2-OCH₃ | 3-OCH₃ |
| 332. | CH₃ | 2-OCH₃ | 4-OCH₃ |
| 333. | CH₃ | 2-OCH₃ | 5-OCH₃ |
| 334. | CH₃ | 3-OCH₃ | 4-OCH₃ |
| 335. | CH₃ | 3-OCH₃ | 5-OCH₃ |
| 336. | CH₃ | 3-OCH₃ | 4,5-(OCH₃)₂ |
| 337. | CH₃ | 2-OC₂H₅ | — |
| 338. | CH₃ | 3-OC₂H₅ | — |
| 339. | CH₃ | 4-OC₂H₅ | — |
| 340. | CH₃ | 2-O-(n-C₃H₇) | — |
| 341. | CH₃ | 3-O-(n-C₃H₇) | — |
| 342. | CH₃ | 4-O-(n-C₃H₇) | — |
| 343. | CH₃ | 2-O-(i-C₃H₇) | — |
| 344. | CH₃ | 3-O-(i-C₃H₇) | — |
| 345. | CH₃ | 4-O-(i-C₃H₇) | — |
| 346. | CH₃ | 4-O-(n-C₄H₉) | — |
| 347. | CH₃ | 3-O-(t-C₄H₉) | — |
| 348. | CH₃ | 4-O-(t-C₄H₉) | — |
| 349. | CH₃ | 2-O-allyl | — |
| 350. | CH₃ | 3-O-allyl | — |
| 351. | CH₃ | 4-O-allyl | — |
| 352. | CH₃ | 2-CF₃ | — |
| 353. | CH₃ | 3-CF₃ | — |
| 354. | CH₃ | 4-CF₃ | — |
| 355. | CH₃ | 2-acetyl | — |
| 356. | CH₃ | 3-acetyl | — |
| 357. | CH₃ | 4-acetyl | — |
| 358. | CH₃ | 2-methoxycarbonyl | — |
| 359. | CH₃ | 3-methoxycarbonyl | — |
| 360. | CH₃ | 4-methoxycarbonyl | — |
| 361. | CH₃ | 2-aminocarbonyl | — |
| 362. | CH₃ | 3-aminocarbonyl | — |
| 363. | CH₃ | 4-aminocarbonyl | — |
| 364. | CH₃ | 2-dimethylaminocarbonyl | — |
| 365. | CH₃ | 3-dimethylaminocarbonyl | — |
| 366. | CH₃ | 4-dimethylaminocarbonyl | — |
| 367. | CH₃ | 2-(N-methylaminocarbonyl) | — |
| 368. | CH₃ | 3-(N-methylaminocarbonyl) | — |
| 369. | CH₃ | 4-(N-methylaminocarbonyl) | — |
| 370. | CH₃ | 2-H₂N | — |
| 371. | CH₃ | 3-H₂N | — |
| 372. | CH₃ | 4-H₂N | — |
| 373. | CH₃ | 2-aminothiocarbonyl | — |
| 374. | CH₃ | 3-aminothiocarbonyl | — |
| 375. | CH₃ | 4-aminothiocarbonyl | — |

| | | |
|---|---|---|
| 376. | CH₃ | 3,4-methylenedioxy |
| 377. | CH₃ | 3,4-difluoromethylenedioxy |
| 378. | CH₃ | 2,3-methylenedioxy |

| | | | |
|---|---|---|---|
| 379. | CH₃ | 2-SCH₃ | — |
| 380. | CH₃ | 3-SCH₃ | — |
| 381. | CH₃ | 4-SCH₃ | — |
| 382. | CH₃ | 2-SO₂CH₃ | — |
| 383. | CH₃ | 3-SO₂CH₃ | — |
| 384. | CH₃ | 4-SO₂CH₃ | — |
| 385. | CH₃ | 2-OCF₃ | — |
| 386. | CH₃ | 3-OCF₃ | — |
| 387. | CH₃ | 4-OCF₃ | — |
| 388. | CH₃ | 2-OCHF₂ | — |
| 389. | CH₃ | 3-OCHF₂ | — |
| 390. | CH₃ | 4-OCHF₂ | — |
| 391. | CH₃ | 3-CF₃ | 4-OCF₃ |
| 392. | CH₃ | 2-NHCH₃ | — |
| 393. | CH₃ | 3-NHCH₃ | — |
| 394. | CH₃ | 4-NHCH₃ | — |
| 395. | CH₃ | 2-N(CH₃)₂ | — |
| 396. | CH₃ | 3-N(CH₃)₂ | — |
| 397. | CH₃ | 4-N(CH₃)₂ | — |
| 398. | CH₃ | 2-ethoxycarbonyl | — |
| 399. | CH₃ | 3-ethoxycarbonyl | — |
| 400. | CH₃ | 4-ethoxycarbonyl | — |
| 401. | CH₃ | 2-CH₂CH₂F | — |
| 402. | CH₃ | 3-CH₂CH₂F | — |
| 403. | CH₃ | 4-CH₂CH₂F | — |
| 404. | CH₃ | 2-CH₂CF₃ | — |
| 405. | CH₃ | 3-CH₂CF₃ | — |
| 406. | CH₃ | 4-CH₂CF₃ | — |
| 407. | CH₃ | 2-CF₂CHF₂ | — |
| 408. | CH₃ | 3-CF₂CHF₂ | — |
| 409. | CH₃ | 4-CF₂CHF₂ | — |
| 410. | CH₃ | 2-CHF₂ | — |
| 411. | CH₃ | 3-CHF₂ | — |
| 412. | CH₃ | 4-CHF₂ | — |
| 413. | CH₃ | 2-(1′-oxo-n-prop-1-yl) | — |
| 414. | CH₃ | 3-(1′-oxo-n-prop-1-yl) | — |
| 415. | CH₃ | 4-(1′-oxo-n-prop-1-yl) | — |
| 416. | CH₃ | 2-(1′-oxoisoprop-1-yl) | — |
| 417. | CH₃ | 3-(1′-oxoisoprop-1-yl) | — |
| 418. | CH₃ | 4-(1′-oxoisoprop-1-yl) | — |
| 419. | CH₃ | 3-cyclopropyl | — |
| 420. | CH₃ | 4-cyclopropyl | — |
| 421. | CH₃ | 4-cyclohexyl | — |
| 422. | CH₃ | 2-methoxyiminomethyl | — |
| 423. | CH₃ | 3-methoxyiminomethyl | — |
| 424. | CH₃ | 4-methoxyiminomethyl | — |
| 425. | CH₃ | 2-ethoxyiminomethyl | — |
| 426. | CH₃ | 3-ethoxyiminomethyl | — |
| 427. | CH₃ | 4-ethoxyiminomethyl | — |
| 428. | CH₃ | 2-isopropyloxyiminomethyl | — |
| 429. | CH₃ | 3-isopropyloxyiminomethyl | — |
| 430. | CH₃ | 4-isopropyloxyiminomethyl | — |
| 431. | CH₃ | 2-allyloxyiminomethyl | — |
| 432. | CH₃ | 3-allyloxyiminomethyl | — |
| 433. | CH₃ | 4-allyloxyiminomethyl | — |
| 434. | CH₃ | 2-phenoxyiminomethyl | — |
| 435. | CH₃ | 3-phenoxyiminomethyl | — |
| 436. | CH₃ | 4-phenoxyiminomethyl | — |
| 437. | CH₃ | 2-benzyloxyiminomethyl | — |
| 438. | CH₃ | 3-benzyloxyiminomethyl | — |
| 439. | CH₃ | 4-benzyloxyiminomethyl | — |
| 440. | CH₃ | 2-(1-methoxyiminoeth-1-yl) | — |
| 441. | CH₃ | 3-(1-methoxyiminoeth-1-yl) | — |
| 442. | CH₃ | 4-(1-methoxyiminoeth-1-yl) | — |
| 443. | CH₃ | 2-(1-isopropyloxyiminoeth-1-yl) | — |
| 444. | CH₃ | 3-(1-isopropyloxyiminoeth-1-yl) | — |
| 445. | CH₃ | 4-(1-isopropyloxyiminoeth-1-yl) | — |
| 446. | CH₃ | 2-(1-allyloxyiminoeth-1-yl) | — |
| 447. | CH₃ | 3-(1-allyloxyiminoeth-1-yl) | — |
| 448. | CH₃ | 4-(1-allyloxyiminoeth-1-yl) | — |
| 449. | CH₃ | 2-(1-phenoxyiminoeth-1-yl) | — |
| 450. | CH₃ | 3-(1-phenoxyiminoeth-1-yl) | — |
| 451. | CH₃ | 4-(1-phenoxyiminoeth-1-yl) | — |
| 452. | CH₃ | 2-(1-benzyloxyiminoeth-1-yl) | — |
| 453. | CH₃ | 3-(1-benzyloxyiminoeth-1-yl) | — |
| 454. | CH₃ | 4-(1-benzyloxyiminoeth-1-yl) | — |
| 455. | CH₃ | 2-(1-ethoxyimino-n-prop-1-yl)- | — |
| 456. | CH₃ | 3-(1-ethoxyimino-n-prop-1-yl)- | — |
| 457. | CH₃ | 4-(1-ethoxyimino-n-prop-1-yl) | — |
| 458. | CH₃ | 2-(1-allyloxyimino-n-prop-1-yl) | — |
| 459. | CH₃ | 3-(1-allyloxyimino-n-prop-1-yl) | — |
| 460. | CH₃ | 4-(1-allyloxyimino-n-prop-1-yl) | — |
| 461. | CF₃ | H | — |
| 462. | CF₃ | 2-F | — |
| 463. | CF₃ | 3-F | — |
| 464. | CF₃ | 4-F | — |
| 465. | CF₃ | 2-F | 3-F |
| 466. | CF₃ | 2-F | 4-F |
| 467. | CF₃ | 2-F | 5-F |
| 468. | CF₃ | 2-F | 6-F |
| 469. | CF₃ | 3-F | 4-F |
| 470. | CF₃ | 3-F | 5-F |
| 471. | CF₃ | 2-Cl | — |
| 472. | CF₃ | 3-Cl | — |
| 473. | CF₃ | 4-Cl | — |
| 474. | CF₃ | 2-Cl | 3-Cl |
| 475. | CF₃ | 2-Cl | 4-Cl |
| 476. | CF₃ | 2-Cl | 5-Cl |
| 477. | CF₃ | 2-Cl | 6-Cl |
| 478. | CF₃ | 3-Cl | 4-Cl |
| 479. | CF₃ | 3-Cl | 5-Cl |
| 480. | CF₃ | 2-Cl | 3,4-Cl₂ |
| 481. | CF₃ | 2-Cl | 3,5-Cl₂ |
| 482. | CF₃ | 2-Cl | 3,6-Cl₂ |
| 483. | CF₃ | 2-Cl | 4,5-Cl₂ |
| 484. | CF₃ | 2-Cl | 4,6-Cl₂ |
| 485. | CF₃ | 3-Cl | 4,5-Cl₂ |
| 486. | CF₃ | 2-Br | — |
| 487. | CF₃ | 3-Br | — |
| 488. | CF₃ | 4-Br | — |
| 489. | CF₃ | 2-Br | 3-Br |
| 490. | CF₃ | 2-Br | 4-Br |
| 491. | CF₃ | 2-Br | 5-Br |
| 492. | CF₃ | 2-Br | 6-Br |
| 493. | CF₃ | 3-Br | 4-Br |
| 494. | CF₃ | 3-Br | 5-Br |
| 495. | CF₃ | 2-F | 3-Cl |
| 496. | CF₃ | 2-F | 4-Cl |
| 497. | CF₃ | 2-F | 5-Cl |
| 498. | CF₃ | 2-F | 3-Br |
| 499. | CF₃ | 2-F | 4-Br |
| 500. | CF₃ | 2-F | 5-Br |
| 501. | CF₃ | 2-Cl | 3-Br |
| 502. | CF₃ | 2-Cl | 4-Br |
| 503. | CF₃ | 2-Cl | 5-Br |
| 504. | CF₃ | 3-F | 4-Cl |
| 505. | CF₃ | 3-F | 5-Cl |
| 506. | CF₃ | 3-F | 6-Cl |
| 507. | CF₃ | 3-F | 4-Br |
| 508. | CF₃ | 3-F | 5-Br |
| 509. | CF₃ | 3-F | 6-Br |
| 510. | CF₃ | 3-Cl | 4-Br |
| 511. | CF₃ | 3-Cl | 5-Br |
| 512. | CF₃ | 3-Cl | 6-Br |
| 513. | CF₃ | 4-F | 5-Cl |
| 514. | CF₃ | 4-F | 6-Cl |
| 515. | CF₃ | 4-F | 5-Br |
| 516. | CF₃ | 4-F | 6-Br |
| 517. | CF₃ | 4-Cl | 5-Br |
| 518. | CF₃ | 5-F | 6-Cl |
| 519. | CF₃ | 5-Fr | 6-Br |
| 520. | CF₃ | 5-Cl | 6-Br |
| 521. | CF₃ | 3-Br | 4-Cl, 5-Br |
| 522. | CF₃ | 2-CN | — |
| 523. | CF₃ | 3-CN | — |
| 524. | CF₃ | 4-CN | — |
| 525. | CF₃ | 2-NO₂ | — |
| 526. | CF₃ | 3-NO₂ | — |
| 527. | CF₃ | 4-NO₂ | — |
| 528. | CF₃ | 2-CH₃ | — |
| 529. | CF₃ | 3-CH₃ | — |
| 530. | CF₃ | 4-CH₃ | — |
| 531. | CF₃ | 2-CH₃ | 3-CH₃ |
| 532. | CF₃ | 2-CH₃ | 4-CH₃ |
| 533. | CF₃ | 2-CH₃ | 5-CH₃ |
| 534. | CF₃ | 2-CH₃ | 6-CH₃ |
| 535. | CF₃ | 3-CH₃ | 4-CH₃ |
| 536. | CF₃ | 3-CH₃ | 5-CH₃ |
| 537. | CF₃ | 2-C₂H₅ | — |
| 538. | CF₃ | 3-C₂H₅ | — |
| 539. | CF₃ | 4-C₂H₅ | — |
| 540. | CF₃ | 2-i-C₃H₇ | — |
| 541. | CF₃ | 3-i-C₃H₇ | — |
| 542. | CF₃ | 4-i-C₃H₇ | — |
| 543. | CF₃ | 3-tert-C₄H₉ | — |
| 544. | CF₃ | 4-tert-C₄H₉ | — |
| 545. | CF₃ | 2-vinyl | — |
| 546. | CF₃ | 3-vinyl | — |
| 547. | CF₃ | 4-vinyl | — |
| 548. | CF₃ | 2-allyl | — |
| 549. | CF₃ | 3-allyl | — |
| 550. | CF₃ | 4-allyl | — |
| 551. | CF₃ | 2-C₆H₅ | — |
| 552. | CF₃ | 3-C₆H₅ | — |
| 553. | CF₃ | 4-C₆H₅ | — |
| 554. | CF₃ | 3-CH₃ | 5-tert-C₄H₉ |
| 555. | CF₃ | 2-OH | — |
| 556. | CF₃ | 3-OH | — |
| 557. | CF₃ | 4-OH | — |
| 558. | CF₃ | 2-OCH₃ | — |
| 559. | CF₃ | 3-OCH₃ | — |
| 560. | CF₃ | 4-OCH₃ | — |
| 561. | CF₃ | 2-OCH₃ | 3-OCH₃ |
| 562. | CF₃ | 2-OCH₃ | 4-OCH₃ |
| 563. | CF₃ | 2-OCH₃ | 5-OCH₃ |
| 564. | CF₃ | 3-OCH₃ | 4-OCH₃ |
| 565. | CF₃ | 3-OCH₃ | 5-OCH₃ |
| 566. | CF₃ | 3-OCH₃ | 4,5-(OCH₃)₂ |
| 567. | CF₃ | 2-OC₂H₅ | — |
| 568. | CF₃ | 3-OC₂H₅ | — |
| 569. | CF₃ | 4-OC₂H₅ | — |
| 570. | CF₃ | 2-O-(n-C₃H₇) | — |
| 571. | CF₃ | 3-O-(n-C₃H₇) | — |
| 572. | CF₃ | 4-O-(n-C₃H₇) | — |
| 573. | CF₃ | 2-O-(i-C₃H₇) | — |
| 574. | CF₃ | 3-O-(i-C₃H₇) | — |
| 575. | CF₃ | 4-O-(i-C₃H₇) | — |
| 576. | CF₃ | 4-O-(n-C₄H₉) | — |
| 577. | CF₃ | 3-O-(t-C₄H₉) | — |
| 578. | CF₃ | 4-O-(t-C₄H₉) | — |
| 579. | CF₃ | 2-O-allyl | — |
| 580. | CF₃ | 3-O-allyl | — |
| 581. | CF₃ | 4-O-allyl | — |
| 582. | CF₃ | 2-CF₃ | — |
| 583. | CF₃ | 3-CF₃ | — |
| 584. | CF₃ | 4-CF₃ | — |
| 585. | CF₃ | 2-acetyl | — |
| 586. | CF₃ | 3-acetyl | — |
| 587. | CF₃ | 4-acetyl | — |
| 588. | CF₃ | 2-methoxycarbonyl | — |
| 589. | CF₃ | 3-methoxycarbonyl | — |
| 590. | CF₃ | 4-methoxycarbonyl | — |
| 591. | CF₃ | 2-aminocarbonyl | — |
| 592. | CF₃ | 3-aminocarbonyl | — |
| 593. | CF₃ | 4-aminocarbonyl | — |
| 594. | CF₃ | 2-dimethylaminocarbonyl | — |
| 595. | CF₃ | 3-dimethylaminocarbonyl | — |
| 596. | CF₃ | 4-dimethylaminocarbonyl | — |
| 597. | CF₃ | 2-(N-methylaminocarbonyl) | — |
| 598. | CF₃ | 3-(N-methylaminocarbonyl) | — |
| 599. | CF₃ | 4-(N-methylaminocarbonyl) | — |
| 600. | CF₃ | 2-H₂N | — |
| 601. | CF₃ | 3-H₂N | — |
| 602. | CF₃ | 4-H₂N | — |
| 603. | CF₃ | 2-aminothiocarbonyl | — |
| 604. | CF₃ | 3-aminothiocarbonyl | — |
| 605. | CF₃ | 4-aminothiocarbonyl | — |

| | | |
|---|---|---|
| 606. | CF₃ | 3,4-methylenedioxy |
| 607. | CF₃ | 3,4-difluoromethylenedioxy |
| 608. | CF₃ | 2,3-methylenedioxy |

| | | | |
|---|---|---|---|
| 609. | CF₃ | 2-SCH₃ | — |
| 610. | CF₃ | 3-SCH₃ | — |
| 611. | CF₃ | 4-SCH₃ | — |
| 612. | CF₃ | 2-SO₂CH₃ | — |
| 613. | CF₃ | 3-SO₂CH₃ | — |
| 614. | CF₃ | 4-SO₂CH₃ | — |
| 615. | CF₃ | 2-OCF₃ | — |
| 616. | CF₃ | 3-OCF₃ | — |
| 617. | CF₃ | 4-OCF₃ | — |
| 618. | CF₃ | 2-OCHF₂ | — |
| 619. | CF₃ | 3-OCHF₂ | — |
| 620. | CF₃ | 4-OCHF₂ | — |
| 621. | CF₃ | 3-CF₃ | 4-OCF₃ |
| 622. | CF₃ | 2-NHCH₃ | — |
| 623. | CF₃ | 3-NHCH₃ | — |
| 624. | CF₃ | 4-NHCH₃ | — |
| 625. | CF₃ | 2-N(CH₃)₂ | — |
| 626. | CF₃ | 3-N(CH₃)₂ | — |
| 627. | CF₃ | 4-N(CH₃)₂ | — |
| 628. | CF₃ | 2-ethoxycarbonyl | — |
| 629. | CF₃ | 3-ethoxycarbonyl | — |
| 630. | CF₃ | 4-ethoxycarbonyl | — |
| 631. | CF₃ | 2-CH₂CH₂F | — |
| 632. | CF₃ | 3-CH₂CH₂F | — |
| 633. | CF₃ | 4-CH₂CH₂F | — |
| 634. | CF₃ | 2-CH₂CF₃ | — |
| 635. | CF₃ | 3-CH₂CF₃ | — |
| 636. | CF₃ | 4-CH₂CF₃ | — |
| 637. | CF₃ | 2-CF₂CHF₂ | — |
| 638. | CF₃ | 3-CF₂CHF₂ | — |
| 639. | CF₃ | 4-CF₂CHF₂ | — |
| 640. | CF₃ | 2-CHF₂ | — |
| 641. | CF₃ | 3-CHF₂ | — |
| 642. | CF₃ | 4-CHF₂ | — |
| 643. | CF₃ | 2-(1′-oxo-n-prop-1-yl) | — |
| 644. | CF₃ | 3-(1′-oxo-n-prop-1-yl) | — |
| 645. | CF₃ | 4-(1′-oxo-n-prop-1-yl) | — |
| 646. | CF₃ | 2-(1′-oxoisoprop-1-yl) | — |
| 647. | CF₃ | 3-(1′-oxoisoprop-1-yl) | — |
| 648. | CF₃ | 4-(1′-oxoisoprop-1-yl) | — |
| 649. | CF₃ | 3-cyclopropyl | — |
| 650. | CF₃ | 4-cyclopropyl | — |
| 651. | CF₃ | 4-cyclohexyl | — |
| 652. | CF₃ | 2-methoxyiminomethyl | — |
| 653. | CF₃ | 3-methoxyiminomethyl | — |
| 654. | CF₃ | 4-methoxyiminomethyl | — |
| 655. | CF₃ | 2-ethoxyiminomethyl | — |
| 656. | CF₃ | 3-ethoxyiminomethyl | — |
| 657. | CF₃ | 4-ethoxyiminomethyl | — |
| 658. | CF₃ | 2-isopropyloxyiminomethyl | — |
| 659. | CF₃ | 3-isopropyloxyiminomethyl | — |
| 660. | CF₃ | 4-isopropyloxyiminomethyl | — |
| 661. | CF₃ | 2-allyloxyiminomethyl | — |
| 662. | CF₃ | 3-allyloxyiminomethyl | — |
| 663. | CF₃ | 4-allyloxyiminomethyl | — |
| 664. | CF₃ | 2-phenoxyiminomethyl | — |
| 665. | CF₃ | 3-phenoxyiminomethyl | — |
| 666. | CF₃ | 4-phenoxyiminomethyl | — |
| 667. | CF₃ | 2-benzyloxyiminomethyl | — |
| 668. | CF₃ | 3-benzyloxyiminomethyl | — |
| 669. | CF₃ | 4-benzyloxyiminomethyl | — |
| 670. | CF₃ | 2-(1-methoxyiminoeth-1-yl) | — |
| 671. | CF₃ | 3-(1-methoxyiminoeth-1-yl) | — |
| 672. | CF₃ | 4-(1-methoxyiminoeth-1-yl) | — |
| 673. | CF₃ | 2-(1-isopropyloxyiminoeth-1-yl) | — |
| 674. | CF₃ | 3-(1-isopropyloxyiminoeth-1-yl) | — |
| 675. | CF₃ | 4-(1-isopropyloxyiminoeth-1-yl) | — |
| 676. | CF₃ | 2-(1-allyloxyiminoeth-1-yl) | — |
| 677. | CF₃ | 3-(1-allyloxyiminoeth-1-yl) | — |
| 678. | CF₃ | 4-(1-allyloxyiminoeth-1-yl) | — |
| 679. | CF₃ | 2-(1-phenoxyiminoeth-1-yl) | — |
| 680. | CF₃ | 3-(1-phenoxyiminoeth-1-yl) | — |
| 681. | CF₃ | 4-(1-phenoxyiminoeth-1-yl) | — |
| 682. | CF₃ | 2-(1-benzyloxyiminoeth-1-yl) | — |
| 683. | CF₃ | 3-(1-benzyloxyiminoeth-1-yl) | — |
| 684. | CF₃ | 4-(1-benzyloxyiminoeth-1-yl) | — |
| 685. | CF₃ | 2-(1-ethoxyimino-n-prop-1-yl)- | — |
| 686. | CF₃ | 3-(1-ethoxyimino-n-prop-1-yl)- | — |
| 687. | CF₃ | 4-(1-ethoxyimino-n-prop-1-yl) | — |
| 688. | CF₃ | 2-(1-allyloxyimino-n-prop-1-yl) | — |
| 689. | CF₃ | 3-(1-allyloxyimino-n-prop-1-yl) | — |
| 690. | CF₃ | 4-(1-allyloxyimino-n-prop-1-yl) | — |
| 691. | CN | H | — |
| 692. | CN | 2-F | — |
| 693. | CN | 3-F | — |
| 694. | CN | 4-F | — |
| 695. | CN | 2-F | 3-F |
| 696. | CN | 2-F | 4-F |
| 697. | CN | 2-F | 5-F |
| 698. | CN | 2-F | 6-F |
| 699. | CN | 3-F | 4-F |
| 700. | CN | 3-F | 5-F |
| 701. | CN | 2-Cl | — |
| 702. | CN | 3-Cl | — |
| 703. | CN | 4-Cl | — |
| 704. | CN | 2-Cl | 3-Cl |
| 705. | CN | 2-Cl | 4-Cl |
| 706. | CN | 2-Cl | 5-Cl |
| 707. | CN | 2-Cl | 6-Cl |
| 708. | CN | 3-Cl | 4-Cl |
| 709. | CN | 3-Cl | 5-Cl |
| 710. | CN | 2-Cl | 3,4-Cl₂ |
| 711. | CN | 2-Cl | 3,5-Cl₂ |
| 712. | CN | 2-Cl | 3,6-Cl₂ |
| 713. | CN | 2-Cl | 4,5-Cl₂ |
| 714. | CN | 2-Cl | 4,6-Cl₂ |
| 715. | CN | 3-Cl | 4,5-Cl₂ |
| 716. | CN | 2-Br | — |
| 717. | CN | 3-Br | — |
| 718. | CN | 4-Br | — |
| 719. | CN | 2-Br | 3-Br |
| 720. | CN | 2-Br | 4-Br |
| 721. | CN | 2-Br | 5-Br |
| 722. | CN | 2-Br | 6-Br |
| 723. | CN | 3-Br | 4-Br |
| 724. | CN | 3-Br | 5-Br |
| 725. | CN | 2-F | 3-Cl |
| 726. | CN | 2-F | 4-Cl |
| 727. | CN | 2-F | 5-Cl |
| 728. | CN | 2-F | 3-Br |
| 729. | CN | 2-F | 4-Br |
| 730. | CN | 2-F | 5-Br |
| 731. | CN | 2-Cl | 3-Br |
| 732. | CN | 2-Cl | 4-Br |
| 733. | CN | 2-Cl | 5-Br |
| 734. | CN | 3-F | 4-Cl |
| 735. | CN | 3-F | 5-Cl |
| 736. | CN | 3-F | 6-Cl |
| 737. | CN | 3-F | 4-Br |
| 738. | CN | 3-F | 5-Br |
| 739. | CN | 3-F | 6-Br |
| 740. | CN | 3-Cl | 4-Br |
| 741. | CN | 3-Cl | 5-Br |
| 742. | CN | 3-Cl | 6-Br |
| 743. | CN | 4-F | 5-Cl |
| 744. | CN | 4-F | 6-Cl |
| 745. | CN | 4-F | 5-Br |
| 746. | CN | 4-F | 6-Br |
| 747. | CN | 4-Cl | 5-Br |
| 748. | CN | 5-F | 6-Cl |
| 749. | CN | 5-Fr | 6-Br |
| 750. | CN | 5-Cl | 6-Br |
| 751. | CN | 3-Br | 4-Cl, 5-Br |
| 752. | CN | 2-CN | — |
| 753. | CN | 3-CN | — |
| 754. | CN | 4-CN | — |
| 755. | CN | 2-NO₂ | — |
| 756. | CN | 3-NO₂ | — |
| 757. | CN | 4-NO₂ | — |
| 758. | CN | 2-CH₃ | — |
| 759. | CN | 3-CH₃ | — |
| 760. | CN | 4-CH₃ | — |
| 761. | CN | 2-CH₃ | 3-CH₃ |
| 762. | CN | 2-CH₃ | 4-CH₃ |
| 763. | CN | 2-CH₃ | 5-CH₃ |
| 764. | CN | 2-CH₃ | 6-CH₃ |
| 765. | CN | 3-CH₃ | 4-CH₃ |
| 766. | CN | 3-CH₃ | 5-CH₃ |
| 767. | CN | 2-C₂H₅ | — |
| 768. | CN | 3-C₂H₅ | — |
| 769. | CN | 4-C₂H₅ | — |
| 770. | CN | 2-i-C₃H₇ | — |
| 771. | CN | 3-i-C₃H₇ | — |
| 772. | CN | 4-i-C₃H₇ | — |
| 773. | CN | 3-tert-C₄H₉ | — |
| 774. | CN | 4-tert-C₄H₉ | — |
| 775. | CN | 2-vinyl | — |
| 776. | CN | 3-vinyl | — |
| 777. | CN | 4-vinyl | — |
| 778. | CN | 2-allyl | — |
| 779. | CN | 3-allyl | — |
| 780. | CN | 4-allyl | — |
| 781. | CN | 2-C₆H₅ | — |
| 782. | CN | 3-C₆H₅ | — |
| 783. | CN | 4-C₆H₅ | — |
| 784. | CN | 3-CH₃ | 5-tert-C₄H₉ |
| 785. | CN | 2-OH | — |
| 786. | CN | 3-OH | — |
| 787. | CN | 4-OH | — |
| 788. | CN | 2-OCH₃ | — |
| 789. | CN | 3-OCH₃ | — |
| 790. | CN | 4-OCH₃ | — |
| 791. | CN | 2-OCH₃ | 3-OCH₃ |
| 792. | CN | 2-OCH₃ | 4-OCH₃ |
| 793. | CN | 2-OCH₃ | 5-OCH₃ |
| 794. | CN | 3-OCH₃ | 4-OCH₃ |
| 795. | CN | 3-OCH₃ | 5-OCH₃ |
| 796. | CN | 3-OCH₃ | 4,5-(OCH₃)₂ |
| 797. | CN | 2-OC₂H₅ | — |
| 798. | CN | 3-OC₂H₅ | — |
| 799. | CN | 4-OC₂H₅ | — |
| 800. | CN | 2-O-(n-C₃H₇) | — |
| 801. | CN | 3-O-(n-C₃H₇) | — |
| 802. | CN | 4-O-(n-C₃H₇) | — |
| 803. | CN | 2-O-(i-C₃H₇) | — |
| 804. | CN | 3-O-(i-C₃H₇) | — |
| 805. | CN | 4-O-(i-C₃H₇) | — |
| 806. | CN | 4-O-(n-C₄H₉) | — |
| 807. | CN | 3-O-(t-C₄H₉) | — |
| 808. | CN | 4-O-(t-C₄H₉) | — |
| 809. | CN | 2-O-allyl | — |
| 810. | CN | 3-O-allyl | — |
| 811. | CN | 4-O-allyl | — |
| 812. | CN | 2-CF₃ | — |
| 813. | CN | 3-CF₃ | — |
| 814. | CN | 4-CF₃ | — |
| 815. | CN | 2-acetyl | — |
| 816. | CN | 3-acetyl | — |
| 817. | CN | 4-acetyl | — |
| 818. | CN | 2-methoxycarbonyl | — |
| 819. | CN | 3-methoxycarbonyl | — |
| 820. | CN | 4-methoxycarbonyl | — |
| 821. | CN | 2-aminocarbonyl | — |
| 822. | CN | 3-aminocarbonyl | — |
| 823. | CN | 4-aminocarbonyl | — |
| 824. | CN | 2-dimethylaminocarbonyl | — |
| 825. | CN | 3-dimethylaminocarbonyl | — |
| 826. | CN | 4-dimethylaminocarbonyl | — |
| 827. | CN | 2-(N-methylaminocarbonyl) | — |
| 828. | CN | 3-(N-methylaminocarbonyl) | — |
| 829. | CN | 4-(N-methylaminocarbonyl) | — |
| 830. | CN | 2-H₂N | — |
| 831. | CN | 3-H₂N | — |
| 832. | CN | 4-H₂N | — |
| 833. | CN | 2-aminothiocarbonyl | — |
| 834. | CN | 3-aminothiocarbonyl | — |
| 835. | CN | 4-aminothiocarbonyl | — |

| | | |
|---|---|---|
| 836. | CN | 3,4-methylenedioxy |
| 837. | CN | 3,4-difluoromethylenedioxy |
| 838. | CN | 2,3-methylenedioxy |

| | | | |
|---|---|---|---|
| 839. | CN | 2-SCH₃ | — |
| 840. | CN | 3-SCH₃ | — |
| 841. | CN | 4-SCH₃ | — |
| 842. | CN | 2-SO₂CH₃ | — |
| 843. | CN | 3-SO₂CH₃ | — |
| 844. | CN | 4-SO₂CH₃ | — |
| 845. | CN | 2-OCF₃ | — |
| 846. | CN | 3-OCF₃ | — |
| 847. | CN | 4-OCF₃ | — |
| 848. | CN | 2-OCHF₂ | — |
| 849. | CN | 3-OCHF₂ | — |
| 850. | CN | 4-OCHF₂ | — |
| 851. | CN | 3-CF₃ | 4-OCF₃ |
| 852. | CN | 2-NHCH₃ | — |
| 853. | CN | 3-NHCH₃ | — |
| 854. | CN | 4-NHCH₃ | — |
| 855. | CN | 2-N(CH₃)₂ | — |
| 856. | CN | 3-N(CH₃)₂ | — |
| 857. | CN | 4-N(CH₃)₂ | — |
| 858. | CN | 2-ethoxycarbonyl | — |
| 859. | CN | 3-ethoxycarbonyl | — |
| 860. | CN | 4-ethoxycarbonyl | — |
| 861. | CN | 2-CH₂CH₂F | — |
| 862. | CN | 3-CH₂CH₂F | — |
| 863. | CN | 4-CH₂CH₂F | — |
| 864. | CN | 2-CH₂CF₃ | — |
| 865. | CN | 3-CH₂CF₃ | — |
| 866. | CN | 4-CH₂CF₃ | — |
| 867. | CN | 2-CF₂CHF₂ | — |
| 868. | CN | 3-CF₂CHF₂ | — |
| 869. | CN | 4-CF₂CHF₂ | — |
| 870. | CN | 2-CHF₂ | — |
| 871. | CN | 3-CHF₂ | — |
| 872. | CN | 4-CHF₂ | — |
| 873. | CN | 2-(1′-oxo-n-prop-1-yl) | — |
| 874. | CN | 3-(1′-oxo-n-prop-1-yl) | — |
| 875. | CN | 4-(1′-oxo-n-prop-1-yl) | — |
| 876. | CN | 2-(1′-oxoisoprop-1-yl) | — |
| 877. | CN | 3-(1′-oxoisoprop-1-yl) | — |
| 878. | CN | 4-(1′-oxoisoprop-1-yl) | — |
| 879. | CN | 3-cyclopropyl | — |
| 880. | CN | 4-cyclopropyl | — |
| 881. | CN | 4-cyclohexyl | — |
| 882. | CN | 2-methoxyiminomethyl | — |
| 883. | CN | 3-methoxyiminomethyl | — |
| 884. | CN | 4-methoxyiminomethyl | — |
| 885. | CN | 2-ethoxyiminomethyl | — |
| 886. | CN | 3-ethoxyiminomethyl | — |
| 887. | CN | 4-ethoxyiminomethyl | — |
| 888. | CN | 2-isopropyloxyiminomethyl | — |
| 889. | CN | 3-isopropyloxyiminomethyl | — |
| 890. | CN | 4-isopropyloxyiminomethyl | — |
| 891. | CN | 2-allyloxyiminomethyl | — |
| 892. | CN | 3-allyloxyiminomethyl | — |
| 893. | CN | 4-allyloxyiminomethyl | — |
| 894. | CN | 2-phenoxyiminomethyl | — |
| 895. | CN | 3-phenoxyiminomethyl | — |
| 896. | CN | 4-phenoxyiminomethyl | — |
| 897. | CN | 2-benzyloxyiminomethyl | — |
| 898. | CN | 3-benzyloxyiminomethyl | — |
| 899. | CN | 4-benzyloxyiminomethyl | — |
| 900. | CN | 2-(1-methoxyiminoeth-1-yl) | — |
| 901. | CN | 3-(1-methoxyiminoeth-1-yl) | — |
| 902. | CN | 4-(1-methoxyiminoeth-1-yl) | — |
| 903. | CN | 2-(1-isopropyloxyiminoeth-1-yl) | — |
| 904. | CN | 3-(1-isopropyloxyiminoeth-1-yl) | — |
| 905. | CN | 4-(1-isopropyloxyiminoeth-1-yl) | — |
| 906. | CN | 2-(1-allyloxyiminoeth-1-yl) | — |
| 907. | CN | 3-(1-allyloxyiminoeth-1-yl) | — |
| 908. | CN | 4-(1-allyloxyiminoeth-1-yl) | — |
| 909. | CN | 2-(1-phenoxyiminoeth-1-yl) | — |
| 910. | CN | 3-(1-phenoxyiminoeth-1-yl) | — |
| 911. | CN | 4-(1-phenoxyiminoeth-1-yl) | — |
| 912. | CN | 2-(1-benzyloxyiminoeth-1-yl) | — |
| 913. | CN | 3-(1-benzyloxyiminoeth-1-yl) | — |
| 914. | CN | 4-(1-benzyloxyiminoeth-1-yl) | — |
| 915. | CN | 2-(1-ethoxyimino-n-prop-1-yl)- | — |
| 916. | CN | 3-(1-ethoxyimino-n-prop-1-yl)- | — |
| 917. | CN | 4-(1-ethoxyimino-n-prop-1-yl) | — |
| 918. | CN | 2-(1-allyloxyimino-n-prop-1-yl) | — |
| 919. | CN | 3-(1-allyloxyimino-n-prop-1-yl) | — |
| 920. | CN | 4-(1-allyloxyimino-n-prop-1-yl) | — |
| 921. | NO₂ | H | — |
| 922. | NO₂ | 2-F | — |
| 923. | NO₂ | 3-F | — |
| 924. | NO₂ | 4-F | — |
| 925. | NO₂ | 2-F | 3-F |
| 926. | NO₂ | 2-F | 4-F |
| 927. | NO₂ | 2-F | 5-F |
| 928. | NO₂ | 2-F | 6-F |
| 929. | NO₂ | 3-F | 4-F |
| 930. | NO₂ | 3-F | 5-F |
| 931. | NO₂ | 2-Cl | — |
| 932. | NO₂ | 3-Cl | — |
| 933. | NO₂ | 4-Cl | — |
| 934. | NO₂ | 2-Cl | 3-Cl |
| 935. | NO₂ | 2-Cl | 4-Cl |
| 936. | NO₂ | 2-Cl | 5-Cl |
| 937. | NO₂ | 2-Cl | 6-Cl |
| 938. | NO₂ | 3-Cl | 4-Cl |
| 939. | NO₂ | 3-Cl | 5-Cl |
| 940. | NO₂ | 2-Cl | 3,4-Cl₂ |
| 941. | NO₂ | 2-Cl | 3,5-Cl₂ |
| 942. | NO₂ | 2-Cl | 3,6-Cl₂ |
| 943. | NO₂ | 2-Cl | 4,5-Cl₂ |
| 944. | NO₂ | 2-Cl | 4,6-Cl₂ |
| 945. | NO₂ | 3-Cl | 4,5-Cl₂ |
| 946. | NO₂ | 2-Br | — |
| 947. | NO₂ | 3-Br | — |
| 948. | NO₂ | 4-Br | — |
| 949. | NO₂ | 2-Br | 3-Br |
| 950. | NO₂ | 2-Br | 4-Br |
| 951. | NO₂ | 2-Br | 5-Br |
| 952. | NO₂ | 2-Br | 6-Br |
| 953. | NO₂ | 3-Br | 4-Br |
| 954. | NO₂ | 3-Br | 5-Br |
| 955. | NO₂ | 2-F | 3-Cl |
| 956. | NO₂ | 2-F | 4-Cl |
| 957. | NO₂ | 2-F | 5-Cl |
| 958. | NO₂ | 2-F | 3-Br |
| 959. | NO₂ | 2-F | 4-Br |
| 960. | NO₂ | 2-F | 5-Br |
| 961. | NO₂ | 2-Cl | 3-Br |
| 962. | NO₂ | 2-Cl | 4-Br |
| 963. | NO₂ | 2-Cl | 5-Br |
| 964. | NO₂ | 3-F | 4-Cl |
| 965. | NO₂ | 3-F | 5-Cl |
| 966. | NO₂ | 3-F | 6-Cl |
| 967. | NO₂ | 3-F | 4-Br |
| 968. | NO₂ | 3-F | 5-Br |
| 969. | NO₂ | 3-F | 6-Br |
| 970. | NO₂ | 3-Cl | 4-Br |
| 971. | NO₂ | 3-Cl | 5-Br |
| 972. | NO₂ | 3-Cl | 6-Br |
| 973. | NO₂ | 4-F | 5-Cl |
| 974. | NO₂ | 4-F | 6-Cl |
| 975. | NO₂ | 4-F | 5-Br |
| 976. | NO₂ | 4-F | 6-Br |
| 977. | NO₂ | 4-Cl | 5-Br |
| 978. | NO₂ | 5-F | 6-Cl |
| 979. | NO₂ | 5-Fr | 6-Br |
| 980. | NO₂ | 5-Cl | 6-Br |
| 981. | NO₂ | 3-Br | 4-Cl, 5-Br |
| 982. | NO₂ | 2-CN | — |
| 983. | NO₂ | 3-CN | — |
| 984. | NO₂ | 4-CN | — |
| 985. | NO₂ | 2-NO₂ | — |
| 986. | NO₂ | 3-NO₂ | — |
| 987. | NO₂ | 4-NO₂ | — |
| 988. | NO₂ | 2-CH₃ | — |
| 989. | NO₂ | 3-CH₃ | — |
| 990. | NO₂ | 4-CH₃ | — |
| 991. | NO₂ | 2-CH₃ | 3-CH₃ |
| 992. | NO₂ | 2-CH₃ | 4-CH₃ |
| 993. | NO₂ | 2-CH₃ | 5-CH₃ |
| 994. | NO₂ | 2-CH₃ | 6-CH₃ |
| 995. | NO₂ | 3-CH₃ | 4-CH₃ |
| 996. | NO₂ | 3-CH₃ | 5-CH₃ |
| 997. | NO₂ | 2-C₂H₅ | — |
| 998. | NO₂ | 3-C₂H₅ | — |
| 999. | NO₂ | 4-C₂H₅ | — |
| 1000. | NO₂ | 2-i-C₃H₇ | — |
| 1001. | NO₂ | 3-i-C₃H₇ | — |
| 1002. | NO₂ | 4-i-C₃H₇ | — |
| 1003. | NO₂ | 3-tert-C₄H₉ | — |
| 1004. | NO₂ | 4-tert-C₄H₉ | — |
| 1005. | NO₂ | 2-vinyl | — |
| 1006. | NO₂ | 3-vinyl | — |
| 1007. | NO₂ | 4-vinyl | — |
| 1008. | NO₂ | 2-allyl | — |
| 1009. | NO₂ | 3-allyl | — |
| 1010. | NO₂ | 4-allyl | — |
| 1011. | NO₂ | 2-C₆H₅ | — |
| 1012. | NO₂ | 3-C₆H₅ | — |
| 1013. | NO₂ | 4-C₆H₅ | — |
| 1014. | NO₂ | 3-CH₃ | 5-tert-C₄H₉ |
| 1015. | NO₂ | 2-OH | — |
| 1016. | NO₂ | 3-OH | — |
| 1017. | NO₂ | 4-OH | — |
| 1018. | NO₂ | 2-OCH₃ | — |
| 1019. | NO₂ | 3-OCH₃ | — |
| 1020. | NO₂ | 4-OCH₃ | — |
| 1021. | NO₂ | 2-OCH₃ | 3-OCH₃ |
| 1022. | NO₂ | 2-OCH₃ | 4-OCH₃ |
| 1023. | NO₂ | 2-OCH₃ | 5-OCH₃ |
| 1024. | NO₂ | 3-OCH₃ | 4-OCH₃ |
| 1025. | NO₂ | 3-OCH₃ | 5-OCH₃ |
| 1026. | NO₂ | 3-OCH₃ | 4,5-(OCH₃)₂ |
| 1027. | NO₂ | 2-OC₂H₅ | — |
| 1028. | NO₂ | 3-OC₂H₅ | — |
| 1029. | NO₂ | 4-OC₂H₅ | — |
| 1030. | NO₂ | 2-O-(n-C₃H₇) | — |
| 1031. | NO₂ | 3-O-(n-C₃H₇) | — |
| 1032. | NO₂ | 4-O-(n-C₃H₇) | — |
| 1033. | NO₂ | 2-O-(i-C₃H₇) | — |
| 1034. | NO₂ | 3-O-(i-C₃H₇) | — |
| 1035. | NO₂ | 4-O-(i-C₃H₇) | — |
| 1036. | NO₂ | 4-O-(n-C₄H₉) | — |
| 1037. | NO₂ | 3-O-(t-C₄H₉) | — |
| 1038. | NO₂ | 4-O-(t-C₄H₉) | — |
| 1039. | NO₂ | 2-O-allyl | — |
| 1040. | NO₂ | 3-O-allyl | — |
| 1041. | NO₂ | 4-O-allyl | — |
| 1042. | NO₂ | 2-CF₃ | — |
| 1043. | NO₂ | 3-CF₃ | — |
| 1044. | NO₂ | 4-CF₃ | — |
| 1045. | NO₂ | 2-acetyl | — |
| 1046. | NO₂ | 3-acetyl | — |
| 1047. | NO₂ | 4-acetyl | — |
| 1048. | NO₂ | 2-methoxycarbonyl | — |
| 1049. | NO₂ | 3-methoxycarbonyl | — |
| 1050. | NO₂ | 4-methoxycarbonyl | — |
| 1051. | NO₂ | 2-aminocarbonyl | — |
| 1052. | NO₂ | 3-aminocarbonyl | — |
| 1053. | NO₂ | 4-aminocarbonyl | — |
| 1054. | NO₂ | 2-dimethylaminocarbonyl | — |
| 1055. | NO₂ | 3-dimethylaminocarbonyl | — |
| 1056. | NO₂ | 4-dimethylaminocarbonyl | — |
| 1057. | NO₂ | 2-(N-methylaminocarbonyl) | — |
| 1058. | NO₂ | 3-(N-methylaminocarbonyl) | — |
| 1059. | NO₂ | 4-(N-methylaminocarbonyl) | — |
| 1060. | NO₂ | 2-H₂N | — |
| 1061. | NO₂ | 3-H₂N | — |
| 1062. | NO₂ | 4-H₂N | — |
| 1063. | NO₂ | 2-aminothiocarbonyl | — |
| 1064. | NO₂ | 3-aminothiocarbonyl | — |
| 1065. | NO₂ | 4-aminothiocarbonyl | — |

| | | |
|---|---|---|
| 1066. | NO₂ | 3,4-methylenedioxy |
| 1067. | NO₂ | 3,4-difluoromethylenedioxy |
| 1068. | NO₂ | 2,3-methylenedioxy |

| | | | |
|---|---|---|---|
| 1069. | NO₂ | 2-SCH₃ | — |
| 1070. | NO₂ | 3-SCH₃ | — |
| 1071. | NO₂ | 4-SCH₃ | — |
| 1072. | NO₂ | 2-SO₂CH₃ | — |
| 1073. | NO₂ | 3-SO₂CH₃ | — |
| 1074. | NO₂ | 4-SO₂CH₃ | — |
| 1075. | NO₂ | 2-OCF₃ | — |
| 1076. | NO₂ | 3-OCF₃ | — |
| 1077. | NO₂ | 4-OCF₃ | — |
| 1078. | NO₂ | 2-OCHF₂ | — |
| 1079. | NO₂ | 3-OCHF₂ | — |
| 1080. | NO₂ | 4-OCHF₂ | — |
| 1081. | NO₂ | 3-CF₃ | 4-OCF₃ |
| 1082. | NO₂ | 2-NHCH₃ | — |
| 1083. | NO₂ | 3-NHCH₃ | — |
| 1084. | NO₂ | 4-NHCH₃ | — |
| 1085. | NO₂ | 2-N(CH₃)₂ | — |
| 1086. | NO₂ | 3-N(CH₃)₂ | — |
| 1087. | NO₂ | 4-N(CH₃)₂ | — |
| 1088. | NO₂ | 2-ethoxycarbonyl | — |
| 1089. | NO₂ | 3-ethoxycarbonyl | — |
| 1090. | NO₂ | 4-ethoxycarbonyl | — |
| 1091. | NO₂ | 2-CH₂CH₂F | — |
| 1092. | NO₂ | 3-CH₂CH₂F | — |
| 1093. | NO₂ | 4-CH₂CH₂F | — |
| 1094. | NO₂ | 2-CH₂CF₃ | — |
| 1095. | NO₂ | 3-CH₂CF₃ | — |
| 1096. | NO₂ | 4-CH₂CF₃ | — |
| 1097. | NO₂ | 2-CF₂CHF₂ | — |
| 1098. | NO₂ | 3-CF₂CHF₂ | — |
| 1099. | NO₂ | 4-CF₂CHF₂ | — |
| 1100. | NO₂ | 2-CHF₂ | — |
| 1101. | NO₂ | 3-CHF₂ | — |
| 1102. | NO₂ | 4-CHF₂ | — |
| 1103. | NO₂ | 2-(1′-oxo-n-prop-1-yl) | — |
| 1104. | NO₂ | 3-(1′-oxo-n-prop-1-yl) | — |
| 1105. | NO₂ | 4-(1′-oxo-n-prop-1-yl) | — |
| 1106. | NO₂ | 2-(1′-oxoisoprop-1-yl) | — |
| 1107. | NO₂ | 3-(1′-oxoisoprop-1-yl) | — |
| 1108. | NO₂ | 4-(1′-oxoisoprop-1-yl) | — |
| 1109. | NO₂ | 3-cyclopropyl | — |
| 1110. | NO₂ | 4-cyclopropyl | — |
| 1111. | NO₂ | 4-cyclohexyl | — |
| 1112. | NO₂ | 2-methoxyiminomethyl | — |
| 1113. | NO₂ | 3-methoxyiminomethyl | — |
| 1114. | NO₂ | 4-methoxyiminomethyl | — |
| 1115. | NO₂ | 2-ethoxyiminomethyl | — |
| 1116. | NO₂ | 3-ethoxyiminomethyl | — |
| 1117. | NO₂ | 4-ethoxyiminomethyl | — |
| 1118. | NO₂ | 2-isopropyloxyiminomethyl | — |
| 1119. | NO₂ | 3-isopropyloxyiminomethyl | — |
| 1120. | NO₂ | 4-isopropyloxyiminomethyl | — |
| 1121. | NO₂ | 2-allyloxyiminomethyl | — |
| 1122. | NO₂ | 3-allyloxyiminomethyl | — |
| 1123. | NO₂ | 4-allyloxyiminomethyl | — |
| 1124. | NO₂ | 2-phenoxyiminomethyl | — |
| 1125. | NO₂ | 3-phenoxyiminomethyl | — |
| 1126. | NO₂ | 4-phenoxyiminomethyl | — |
| 1127. | NO₂ | 2-benzyloxyiminomethyl | — |
| 1128. | NO₂ | 3-benzyloxyiminomethyl | — |
| 1129. | NO₂ | 4-benzyloxyiminomethyl | — |
| 1130. | NO₂ | 2-(1-methoxyiminoeth-1-yl) | — |
| 1131. | NO₂ | 3-(1-methoxyiminoeth-1-yl) | — |
| 1132. | NO₂ | 4-(1-methoxyiminoeth-1-yl) | — |
| 1133. | NO₂ | 2-(1-isopropyloxyiminoeth-1-yl) | — |
| 1134. | NO₂ | 3-(1-isopropyloxyiminoeth-1-yl) | — |
| 1135. | NO₂ | 4-(1-isopropyloxyiminoeth-1-yl) | — |
| 1136. | NO₂ | 2-(1-allyloxyiminoeth-1-yl) | — |
| 1137. | NO₂ | 3-(1-allyloxyiminoeth-1-yl) | — |
| 1138. | NO₂ | 4-(1-allyloxyiminoeth-1-yl) | — |
| 1139. | NO₂ | 2-(1-phenoxyiminoeth-1-yl) | — |
| 1140. | NO₂ | 3-(1-phenoxyiminoeth-1-yl) | — |
| 1141. | NO₂ | 4-(1-phenoxyiminoeth-1-yl) | — |
| 1142. | NO₂ | 2-(1-benzyloxyiminoeth-1-yl) | — |
| 1143. | NO₂ | 3-(1-benzyloxyiminoeth-1-yl) | — |
| 1144. | NO₂ | 4-(1-benzyloxyiminoeth-1-yl) | — |
| 1145. | NO₂ | 2-(1-ethoxyimino-n-prop-1-yl)- | — |
| 1146. | NO₂ | 3-(1-ethoxyimino-n-prop-1-yl)- | — |
| 1147. | NO₂ | 4-(1-ethoxyimino-n-prop-1-yl) | — |
| 1148. | NO₂ | 2-(1-allyloxyimino-n-prop-1-yl) | — |
| 1149. | NO₂ | 3-(1-allyloxyimino-n-prop-1-yl) | — |
| 1150. | NO₂ | 4-(1-allyloxyimino-n-prop-1-yl) | — |
| 1151. | Cl | H | — |
| 1152. | Cl | 2-F | — |
| 1153. | Cl | 3-F | — |
| 1154. | Cl | 4-F | — |
| 1155. | Cl | 2-F | 3-F |
| 1156. | Cl | 2-F | 4-F |
| 1157. | Cl | 2-F | 5-F |
| 1158. | Cl | 2-F | 6-F |
| 1159. | Cl | 3-F | 4-F |
| 1160. | Cl | 3-F | 5-F |
| 1161. | Cl | 2-Cl | — |
| 1162. | Cl | 3-Cl | — |
| 1163. | Cl | 4-Cl | — |
| 1164. | Cl | 2-Cl | 3-Cl |
| 1165. | Cl | 2-Cl | 4-Cl |
| 1166. | Cl | 2-Cl | 5-Cl |
| 1167. | Cl | 2-Cl | 6-Cl |
| 1168. | Cl | 3-Cl | 4-Cl |
| 1169. | Cl | 3-Cl | 5-Cl |
| 1170. | Cl | 2-Cl | 3,4-Cl₂ |
| 1171. | Cl | 2-Cl | 3,5-Cl₂ |
| 1172. | Cl | 2-Cl | 3,6-Cl₂ |
| 1173. | Cl | 2-Cl | 4,5-Cl₂ |
| 1174. | Cl | 2-Cl | 4,6-Cl₂ |
| 1175. | Cl | 3-Cl | 4,5-Cl₂ |
| 1176. | Cl | 2-Br | — |
| 1177. | Cl | 3-Br | — |
| 1178. | Cl | 4-Br | — |
| 1179. | Cl | 2-Br | 3-Br |
| 1180. | Cl | 2-Br | 4-Br |
| 1181. | Cl | 2-Br | 5-Br |
| 1182. | Cl | 2-Br | 6-Br |
| 1183. | Cl | 3-Br | 4-Br |
| 1184. | Cl | 3-Br | 5-Br |
| 1185. | Cl | 2-F | 3-Cl |
| 1186. | Cl | 2-F | 4-Cl |
| 1187. | Cl | 2-F | 5-Cl |
| 1188. | Cl | 2-F | 3-Br |
| 1189. | Cl | 2-F | 4-Br |
| 1190. | Cl | 2-F | 5-Br |
| 1191. | Cl | 2-Cl | 3-Br |
| 1192. | Cl | 2-Cl | 4-Br |
| 1193. | Cl | 2-Cl | 5-Br |
| 1194. | Cl | 3-F | 4-Cl |
| 1195. | Cl | 3-F | 5-Cl |
| 1196. | Cl | 3-F | 6-Cl |
| 1197. | Cl | 3-F | 4-Br |
| 1198. | Cl | 3-F | 5-Br |
| 1199. | Cl | 3-F | 6-Br |
| 1200. | Cl | 3-Cl | 4-Br |
| 1201. | Cl | 3-Cl | 5-Br |
| 1202. | Cl | 3-Cl | 6-Br |
| 1203. | Cl | 4-F | 5-Cl |
| 1204. | Cl | 4-F | 6-Cl |
| 1205. | Cl | 4-F | 5-Br |
| 1206. | Cl | 4-F | 6-Br |
| 1207. | Cl | 4-Cl | 5-Br |
| 1208. | Cl | 5-F | 6-Cl |
| 1209. | Cl | 5-Fr | 6-Br |
| 1210. | Cl | 5-Cl | 6-Br |
| 1211. | Cl | 3-Br | 4-Cl, 5-Br |
| 1212. | Cl | 2-CN | — |
| 1213. | Cl | 3-CN | — |
| 1214. | Cl | 4-CN | — |
| 1215. | Cl | 2-NO₂ | — |
| 1216. | Cl | 3-NO₂ | — |
| 1217. | Cl | 4-NO₂ | — |
| 1218. | Cl | 2-CH₃ | — |
| 1219. | Cl | 3-CH₃ | — |
| 1220. | Cl | 4-CH₃ | — |
| 1221. | Cl | 2-CH₃ | 3-CH₃ |
| 1222. | Cl | 2-CH₃ | 4-CH₃ |
| 1223. | Cl | 2-CH₃ | 5-CH₃ |
| 1224. | Cl | 2-CH₃ | 6-CH₃ |
| 1225. | Cl | 3-CH₃ | 4-CH₃ |
| 1226. | Cl | 3-CH₃ | 5-CH₃ |
| 1227. | Cl | 2-C₂H₅ | — |
| 1228. | Cl | 3-C₂H₅ | — |
| 1229. | Cl | 4-C₂H₅ | — |
| 1230. | Cl | 2-i-C₃H₇ | — |
| 1231. | Cl | 3-i-C₃H₇ | — |
| 1232. | Cl | 4-i-C₃H₇ | — |
| 1233. | Cl | 3-tert-C₄H₉ | — |
| 1234. | Cl | 4-tert-C₄H₉ | — |
| 1235. | Cl | 2-vinyl | — |
| 1236. | Cl | 3-vinyl | — |
| 1237. | Cl | 4-vinyl | — |
| 1238. | Cl | 2-allyl | — |
| 1239. | Cl | 3-allyl | — |
| 1240. | Cl | 4-allyl | — |
| 1241. | Cl | 2-C₆H₅ | — |
| 1242. | Cl | 3-C₆H₅ | — |
| 1243. | Cl | 4-C₆H₅ | — |
| 1244. | Cl | 3-CH₃ | 5-tert-C₄H₉ |
| 1245. | Cl | 2-OH | — |
| 1246. | Cl | 3-OH | — |
| 1247. | Cl | 4-OH | — |
| 1248. | Cl | 2-OCH₃ | — |
| 1249. | Cl | 3-OCH₃ | — |
| 1250. | Cl | 4-OCH₃ | — |
| 1251. | Cl | 2-OCH₃ | 3-OCH₃ |
| 1252. | Cl | 2-OCH₃ | 4-OCH₃ |
| 1253. | Cl | 2-OCH₃ | 5-OCH₃ |
| 1254. | Cl | 3-OCH₃ | 4-OCH₃ |
| 1255. | Cl | 3-OCH₃ | 5-OCH₃ |
| 1256. | Cl | 3-OCH₃ | 4,5-(OCH₃)₂ |
| 1257. | Cl | 2-OC₂H₅ | — |
| 1258. | Cl | 3-OC₂H₅ | — |
| 1259. | Cl | 4-OC₂H₅ | — |
| 1260. | Cl | 2-O-(n-C₃H₇) | — |
| 1261. | Cl | 3-O-(n-C₃H₇) | — |
| 1262. | Cl | 4-O-(n-C₃H₇) | — |
| 1263. | Cl | 2-O-(i-C₃H₇) | — |
| 1264. | Cl | 3-O-(i-C₃H₇) | — |
| 1265. | Cl | 4-O-(i-C₃H₇) | — |
| 1266. | Cl | 4-O-(n-C₄H₉) | — |
| 1267. | Cl | 3-O-(t-C₄H₉) | — |
| 1268. | Cl | 4-O-(t-C₄H₉) | — |
| 1269. | Cl | 2-O-allyl | — |
| 1270. | Cl | 3-O-allyl | — |
| 1271. | Cl | 4-O-allyl | — |
| 1272. | Cl | 2-CF₃ | — |
| 1273. | Cl | 3-CF₃ | — |
| 1274. | Cl | 4-CF₃ | — |
| 1275. | Cl | 2-acetyl | — |
| 1276. | Cl | 3-acetyl | — |
| 1277. | Cl | 4-acetyl | — |
| 1278. | Cl | 2-methoxycarbonyl | — |
| 1279. | Cl | 3-methoxycarbonyl | — |
| 1280. | Cl | 4-methoxycarbonyl | — |
| 1281. | Cl | 2-aminocarbonyl | — |
| 1282. | Cl | 3-aminocarbonyl | — |
| 1283. | Cl | 4-aminocarbonyl | — |
| 1284. | Cl | 2-dimethylaminocarbonyl | — |
| 1285. | Cl | 3-dimethylaminocarbonyl | — |
| 1286. | Cl | 4-dimethylaminocarbonyl | — |
| 1287. | Cl | 2-(N-methylaminocarbonyl) | — |
| 1288. | Cl | 3-(N-methylaminocarbonyl) | — |
| 1289. | Cl | 4-(N-methylaminocarbonyl) | — |
| 1290. | Cl | 2-H₂N | — |
| 1291. | Cl | 3-H₂N | — |
| 1292. | Cl | 4-H₂N | — |
| 1293. | Cl | 2-aminothiocarbonyl | — |
| 1294. | Cl | 3-aminothiocarbonyl | — |
| 1295. | Cl | 4-aminothiocarbonyl | — |

| | | |
|---|---|---|
| 1296. | Cl | 3,4-methylenedioxy |
| 1297. | Cl | 3,4-difluoromethylenedioxy |
| 1298. | Cl | 2,3-methylenedioxy |

| | | | |
|---|---|---|---|
| 1299. | Cl | 2-SCH₃ | — |
| 1300. | Cl | 3-SCH₃ | — |
| 1301. | Cl | 4-SCH₃ | — |
| 1302. | Cl | 2-SO₂CH₃ | — |
| 1303. | Cl | 3-SO₂CH₃ | — |
| 1304. | Cl | 4-SO₂CH₃ | — |
| 1305. | Cl | 2-OCF₃ | — |
| 1306. | Cl | 3-OCF₃ | — |
| 1307. | Cl | 4-OCF₃ | — |
| 1308. | Cl | 2-OCHF₂ | — |
| 1309. | Cl | 3-OCHF₂ | — |
| 1310. | Cl | 4-OCHF₂ | — |
| 1311. | Cl | 3-CF₃ | 4-OCF₃ |
| 1312. | Cl | 2-NHCH₃ | — |
| 1313. | Cl | 3-NHCH₃ | — |
| 1314. | Cl | 4-NHCH₃ | — |
| 1315. | Cl | 2-N(CH₃)₂ | — |
| 1316. | Cl | 3-N(CH₃)₂ | — |
| 1317. | Cl | 4-N(CH₃)₂ | — |
| 1318. | Cl | 2-ethoxycarbonyl | — |
| 1319. | Cl | 3-ethoxycarbonyl | — |
| 1320. | Cl | 4-ethoxycarbonyl | — |
| 1321. | Cl | 2-CH₂CH₂F | — |
| 1322. | Cl | 3-CH₂CH₂F | — |
| 1323. | Cl | 4-CH₂CH₂F | — |
| 1324. | Cl | 2-CH₂CF₃ | — |
| 1325. | Cl | 3-CH₂CF₃ | — |
| 1326. | Cl | 4-CH₂CF₃ | — |
| 1327. | Cl | 2-CF₂CHF₂ | — |
| 1328. | Cl | 3-CF₂CHF₂ | — |
| 1329. | Cl | 4-CF₂CHF₂ | — |
| 1330. | Cl | 2-CHF₂ | — |
| 1331. | Cl | 3-CHF₂ | — |
| 1332. | Cl | 4-CHF₂ | — |
| 1333. | Cl | 2-(1′-oxo-n-prop-1-yl) | — |
| 1334. | Cl | 3-(1′-oxo-n-prop-1-yl) | — |
| 1335. | Cl | 4-(1′-oxo-n-prop-1-yl) | — |
| 1336. | Cl | 2-(1′-oxoisoprop-1-yl) | — |
| 1337. | Cl | 3-(1′-oxoisoprop-1-yl) | — |
| 1338. | Cl | 4-(1′-oxoisoprop-1-yl) | — |
| 1339. | Cl | 3-cyclopropyl | — |
| 1340. | Cl | 4-cyclopropyl | — |
| 1341. | Cl | 4-cyclohexyl | — |
| 1342. | Cl | 2-methoxyiminomethyl | — |
| 1343. | Cl | 3-methoxyiminomethyl | — |
| 1344. | Cl | 4-methoxyiminomethyl | — |
| 1345. | Cl | 2-ethoxyiminomethyl | — |
| 1346. | Cl | 3-ethoxyiminomethyl | — |
| 1347. | Cl | 4-ethoxyiminomethyl | — |
| 1348. | Cl | 2-isopropyloxyiminomethyl | — |
| 1349. | Cl | 3-isopropyloxyiminomethyl | — |
| 1350. | Cl | 4-isopropyloxyiminomethyl | — |
| 1351. | Cl | 2-allyloxyiminomethyl | — |
| 1352. | Cl | 3-allyloxyiminomethyl | — |
| 1353. | Cl | 4-allyloxyiminomethyl | — |
| 1354. | Cl | 2-phenoxyiminomethyl | — |
| 1355. | Cl | 3-phenoxyiminomethyl | — |
| 1356. | Cl | 4-phenoxyiminomethyl | — |
| 1357. | Cl | 2-benzyloxyiminomethyl | — |
| 1358. | Cl | 3-benzyloxyiminomethyl | — |
| 1359. | Cl | 4-benzyloxyiminomethyl | — |
| 1360. | Cl | 2-(1-methoxyiminoeth-1-yl) | — |
| 1361. | Cl | 3-(1-methoxyiminoeth-1-yl) | — |
| 1362. | Cl | 4-(1-methoxyiminoeth-1-yl) | — |
| 1363. | Cl | 2-(1-isopropyloxyiminoeth-1-yl) | — |
| 1364. | Cl | 3-(1-isopropyloxyiminoeth-1-yl) | — |
| 1365. | Cl | 4-(1-isopropyloxyiminoeth-1-yl) | — |
| 1366. | Cl | 2-(1-allyloxyiminoeth-1-yl) | — |
| 1367. | Cl | 3-(1-allyloxyiminoeth-1-yl) | — |
| 1368. | Cl | 4-(1-allyloxyiminoeth-1-yl) | — |
| 1369. | Cl | 2-(1-phenoxyiminoeth-1-yl) | — |
| 1370. | Cl | 3-(1-phenoxyiminoeth-1-yl) | — |
| 1371. | Cl | 4-(1-phenoxyiminoeth-1-yl) | — |
| 1372. | Cl | 2-(1-benzyloxyiminoeth-1-yl) | — |
| 1373. | Cl | 3-(1-benzyloxyiminoeth-1-yl) | — |
| 1374. | Cl | 4-(1-benzyloxyiminoeth-1-yl) | — |
| 1375. | Cl | 2-(1-ethoxyimino-n-prop-1-yl)- | — |
| 1376. | Cl | 3-(1-ethoxyimino-n-prop-1-yl)- | — |
| 1377. | Cl | 4-(1-ethoxyimino-n-prop-1-yl) | — |
| 1378. | Cl | 2-(1-allyloxyimino-n-prop-1-yl) | — |
| 1379. | Cl | 3-(1-allyloxyimino-n-prop-1-yl) | — |
| 1380. | Cl | 4-(1-allyloxyimino-n-prop-1-yl) | — |
| 1381. | Br | H | — |
| 1382. | Br | 2-F | — |
| 1383. | Br | 3-F | — |
| 1384. | Br | 4-F | — |
| 1385. | Br | 2-F | 3-F |
| 1386. | Br | 2-F | 4-F |
| 1387. | Br | 2-F | 5-F |
| 1388. | Br | 2-F | 6-F |
| 1389. | Br | 3-F | 4-F |
| 1390. | Br | 3-F | 5-F |
| 1391. | Br | 2-Cl | — |
| 1392. | Br | 3-Cl | — |
| 1393. | Br | 4-Cl | — |
| 1394. | Br | 2-Cl | 3-Cl |
| 1395. | Br | 2-Cl | 4-Cl |
| 1396. | Br | 2-Cl | 5-Cl |
| 1397. | Br | 2-Cl | 6-Cl |
| 1398. | Br | 3-Cl | 4-Cl |
| 1399. | Br | 3-Cl | 5-Cl |
| 1400. | Br | 2-Cl | 3,4-Cl₂ |
| 1401. | Br | 2-Cl | 3,5-Cl₂ |
| 1402. | Br | 2-Cl | 3,6-Cl₂ |
| 1403. | Br | 2-Cl | 4,5-Cl₂ |
| 1404. | Br | 2-Cl | 4,6-Cl₂ |
| 1405. | Br | 3-Cl | 4,5-Cl₂ |
| 1406. | Br | 2-Br | — |
| 1407. | Br | 3-Br | — |
| 1408. | Br | 4-Br | — |
| 1409. | Br | 2-Br | 3-Br |
| 1410. | Br | 2-Br | 4-Br |
| 1411. | Br | 2-Br | 5-Br |
| 1412. | Br | 2-Br | 6-Br |
| 1413. | Br | 3-Br | 4-Br |
| 1414. | Br | 3-Br | 5-Br |
| 1415. | Br | 2-F | 3-Cl |
| 1416. | Br | 2-F | 4-Cl |
| 1417. | Br | 2-F | 5-Cl |
| 1418. | Br | 2-F | 3-Br |
| 1419. | Br | 2-F | 4-Br |
| 1420. | Br | 2-F | 5-Br |
| 1421. | Br | 2-Cl | 3-Br |
| 1422. | Br | 2-Cl | 4-Br |
| 1423. | Br | 2-Cl | 5-Br |
| 1424. | Br | 3-F | 4-Cl |
| 1425. | Br | 3-F | 5-Cl |
| 1426. | Br | 3-F | 6-Cl |
| 1427. | Br | 3-F | 4-Br |
| 1428. | Br | 3-F | 5-Br |
| 1429. | Br | 3-F | 6-Br |
| 1430. | Br | 3-Cl | 4-Br |
| 1431. | Br | 3-Cl | 5-Br |
| 1432. | Br | 3-Cl | 6-Br |
| 1433. | Br | 4-F | 5-Cl |
| 1434. | Br | 4-F | 6-Cl |
| 1435. | Br | 4-F | 5-Br |
| 1436. | Br | 4-F | 6-Br |
| 1437. | Br | 4-Cl | 5-Br |
| 1438. | Br | 5-F | 6-Cl |
| 1439. | Br | 5-Fr | 6-Br |
| 1440. | Br | 5-Cl | 6-Br |
| 1441. | Br | 3-Br | 4-Cl, 5-Br |
| 1442. | Br | 2-CN | — |
| 1443. | Br | 3-CN | — |
| 1444. | Br | 4-CN | — |
| 1445. | Br | 2-NO₂ | — |
| 1446. | Br | 3-NO₂ | — |
| 1447. | Br | 4-NO₂ | — |
| 1448. | Br | 2-CH₃ | — |
| 1449. | Br | 3-CH₃ | — |
| 1450. | Br | 4-CH₃ | — |
| 1451. | Br | 2-CH₃ | 3-CH₃ |
| 1452. | Br | 2-CH₃ | 4-CH₃ |
| 1453. | Br | 2-CH₃ | 5-CH₃ |
| 1454. | Br | 2-CH₃ | 6-CH₃ |
| 1455. | Br | 3-CH₃ | 4-CH₃ |
| 1456. | Br | 3-CH₃ | 5-CH₃ |
| 1457. | Br | 2-C₂H₅ | — |
| 1458. | Br | 3-C₂H₅ | — |
| 1459. | Br | 4-C₂H₅ | — |
| 1460. | Br | 2-i-C₃H₇ | — |
| 1461. | Br | 3-i-C₃H₇ | — |
| 1462. | Br | 4-i-C₃H₇ | — |
| 1463. | Br | 3-tert-C₄H₉ | — |
| 1464. | Br | 4-tert-C₄H₉ | — |
| 1465. | Br | 2-vinyl | — |
| 1466. | Br | 3-vinyl | — |
| 1467. | Br | 4-vinyl | — |
| 1468. | Br | 2-allyl | — |
| 1469. | Br | 3-allyl | — |
| 1470. | Br | 4-allyl | — |
| 1471. | Br | 2-C₆H₅ | — |
| 1472. | Br | 3-C₆H₅ | — |
| 1473. | Br | 4-C₆H₅ | — |
| 1474. | Br | 3-CH₃ | 5-tert-C₄H₉ |
| 1475. | Br | 2-OH | — |
| 1476. | Br | 3-OH | — |
| 1477. | Br | 4-OH | — |
| 1478. | Br | 2-OCH₃ | — |
| 1479. | Br | 3-OCH₃ | — |
| 1480. | Br | 4-OCH₃ | — |
| 1481. | Br | 2-OCH₃ | 3-OCH₃ |
| 1482. | Br | 2-OCH₃ | 4-OCH₃ |
| 1483. | Br | 2-OCH₃ | 5-OCH₃ |
| 1484. | Br | 3-OCH₃ | 4-OCH₃ |
| 1485. | Br | 3-OCH₃ | 5-OCH₃ |
| 1486. | Br | 3-OCH₃ | 4,5-(OCH₃)₂ |
| 1487. | Br | 2-OC₂H₅ | — |
| 1488. | Br | 3-OC₂H₅ | — |
| 1489. | Br | 4-OC₂H₅ | — |
| 1490. | Br | 2-O-(n-C₃H₇) | — |
| 1491. | Br | 3-O-(n-C₃H₇) | — |
| 1492. | Br | 4-O-(n-C₃H₇) | — |
| 1493. | Br | 2-O-(i-C₃H₇) | — |
| 1494. | Br | 3-O-(i-C₃H₇) | — |
| 1495. | Br | 4-O-(i-C₃H₇) | — |
| 1496. | Br | 4-O-(n-C₄H₉) | — |
| 1497. | Br | 3-O-(t-C₄H₉) | — |
| 1498. | Br | 4-O-(t-C₄H₉) | — |
| 1499. | Br | 2-O-allyl | — |
| 1500. | Br | 3-O-allyl | — |
| 1501. | Br | 4-O-allyl | — |
| 1502. | Br | 2-CF₃ | — |
| 1503. | Br | 3-CF₃ | — |
| 1504. | Br | 4-CF₃ | — |
| 1505. | Br | 2-acetyl | — |
| 1506. | Br | 3-acetyl | — |
| 1507. | Br | 4-acetyl | — |
| 1508. | Br | 2-methoxycarbonyl | — |
| 1509. | Br | 3-methoxycarbonyl | — |
| 1510. | Br | 4-methoxycarbonyl | — |
| 1511. | Br | 2-aminocarbonyl | — |
| 1512. | Br | 3-aminocarbonyl | — |
| 1513. | Br | 4-aminocarbonyl | — |
| 1514. | Br | 2-dimethylaminocarbonyl | — |
| 1515. | Br | 3-dimethylaminocarbonyl | — |
| 1516. | Br | 4-dimethylaminocarbonyl | — |
| 1517. | Br | 2-(N-methylaminocarbonyl) | — |
| 1518. | Br | 3-(N-methylaminocarbonyl) | — |
| 1519. | Br | 4-(N-methylaminocarbonyl) | — |
| 1520. | Br | 2-H₂N | — |
| 1521. | Br | 3-H₂N | — |
| 1522. | Br | 4-H₂N | — |
| 1523. | Br | 2-aminothiocarbonyl | — |
| 1524. | Br | 3-aminothiocarbonyl | — |
| 1525. | Br | 4-aminothiocarbonyl | — |

| | | |
|---|---|---|
| 1526. | Br | 3,4-methylenedioxy |
| 1527. | Br | 3,4-difluoromethylenedioxy |
| 1528. | Br | 2,3-methylenedioxy |

| | | | |
|---|---|---|---|
| 1529. | Br | 2-SCH₃ | — |
| 1530. | Br | 3-SCH₃ | — |
| 1531. | Br | 4-SCH₃ | — |
| 1532. | Br | 2-SO₂CH₃ | — |
| 1533. | Br | 3-SO₂CH₃ | — |
| 1534. | Br | 4-SO₂CH₃ | — |
| 1535. | Br | 2-OCF₃ | — |
| 1536. | Br | 3-OCF₃ | — |
| 1537. | Br | 4-OCF₃ | — |
| 1538. | Br | 2-OCHF₂ | — |
| 1539. | Br | 3-OCHF₂ | — |
| 1540. | Br | 4-OCHF₂ | — |
| 1541. | Br | 3-CF₃ | 4-OCF₃ |
| 1542. | Br | 2-NHCH₃ | — |
| 1543. | Br | 3-NHCH₃ | — |
| 1544. | Br | 4-NHCH₃ | — |
| 1545. | Br | 2-N(CH₃)₂ | — |
| 1546. | Br | 3-N(CH₃)₂ | — |
| 1547. | Br | 4-N(CH₃)₂ | — |
| 1548. | Br | 2-ethoxycarbonyl | — |
| 1549. | Br | 3-ethoxycarbonyl | — |
| 1550. | Br | 4-ethoxycarbonyl | — |
| 1551. | Br | 2-CH₂CH₂F | — |
| 1552. | Br | 3-CH₂CH₂F | — |
| 1553. | Br | 4-CH₂CH₂F | — |
| 1554. | Br | 2-CH₂CF₃ | — |
| 1555. | Br | 3-CH₂CF₃ | — |
| 1556. | Br | 4-CH₂CF₃ | — |
| 1557. | Br | 2-CF₂CHF₂ | — |
| 1558. | Br | 3-CF₂CHF₂ | — |
| 1559. | Br | 4-CF₂CHF₂ | — |
| 1560. | Br | 2-CHF₂ | — |
| 1561. | Br | 3-CHF₂ | — |
| 1562. | Br | 4-CHF₂ | — |
| 1563. | Br | 2-(1′-oxo-n-prop-1-yl) | — |
| 1564. | Br | 3-(1′-oxo-n-prop-1-yl) | — |
| 1565. | Br | 4-(1′-oxo-n-prop-1-yl) | — |
| 1566. | Br | 2-(1′-oxoisoprop-1-yl) | — |
| 1567. | Br | 3-(1′-oxoisoprop-1-yl) | — |
| 1568. | Br | 4-(1′-oxoisoprop-1-yl) | — |
| 1569. | Br | 3-cyclopropyl | — |
| 1570. | Br | 4-cyclopropyl | — |
| 1571. | Br | 4-cyclohexyl | — |
| 1572. | Br | 2-methoxyiminomethyl | — |
| 1573. | Br | 3-methoxyiminomethyl | — |
| 1574. | Br | 4-methoxyiminomethyl | — |
| 1575. | Br | 2-ethoxyiminomethyl | — |
| 1576. | Br | 3-ethoxyiminomethyl | — |
| 1577. | Br | 4-ethoxyiminomethyl | — |
| 1578. | Br | 2-isopropyloxyiminomethyl | — |
| 1579. | Br | 3-isopropyloxyiminomethyl | — |
| 1580. | Br | 4-isopropyloxyiminomethyl | — |
| 1581. | Br | 2-allyloxyiminomethyl | — |
| 1582. | Br | 3-allyloxyiminomethyl | — |
| 1583. | Br | 4-allyloxyiminomethyl | — |
| 1584. | Br | 2-phenoxyiminomethyl | — |
| 1585. | Br | 3-phenoxyiminomethyl | — |
| 1586. | Br | 4-phenoxyiminomethyl | — |
| 1587. | Br | 2-benzyloxyiminomethyl | — |
| 1588. | Br | 3-benzyloxyiminomethyl | — |
| 1589. | Br | 4-benzyloxyiminomethyl | — |
| 1590. | Br | 2-(1-methoxyiminoeth-1-yl) | — |
| 1591. | Br | 3-(1-methoxyiminoeth-1-yl) | — |
| 1592. | Br | 4-(1-methoxyiminoeth-1-yl) | — |
| 1593. | Br | 2-(1-isopropyloxyiminoeth-1-yl) | — |
| 1594. | Br | 3-(1-isopropyloxyiminoeth-1-yl) | — |
| 1595. | Br | 4-(1-isopropyloxyiminoeth-1-yl) | — |
| 1596. | Br | 2-(1-allyloxyiminoeth-1-yl) | — |
| 1597. | Br | 3-(1-allyloxyiminoeth-1-yl) | — |
| 1598. | Br | 4-(1-allyloxyiminoeth-1-yl) | — |
| 1599. | Br | 2-(1-phenoxyiminoeth-1-yl) | — |
| 1600. | Br | 3-(1-phenoxyiminoeth-1-yl) | — |
| 1601. | Br | 4-(1-phenoxyiminoeth-1-yl) | — |
| 1602. | Br | 2-(1-benzyloxyiminoeth-1-yl) | — |
| 1603. | Br | 3-(1-benzyloxyiminoeth-1-yl) | — |
| 1604. | Br | 4-(1-benzyloxyiminoeth-1-yl) | — |
| 1605. | Br | 2-(1-ethoxyimino-n-prop-1-yl)- | — |
| 1606. | Br | 3-(1-ethoxyimino-n-prop-1-yl)- | — |
| 1607. | Br | 4-(1-ethoxyimino-n-prop-1-yl) | — |
| 1608. | Br | 2-(1-allyloxyimino-n-prop-1-yl) | — |
| 1609. | Br | 3-(1-allyloxyimino-n-prop-1-yl) | — |
| 1610. | Br | 4-(1-allyloxyimino-n-prop-1-yl) | — |
| 1611. | I | H | — |
| 1612. | I | 2-F | — |
| 1613. | I | 3-F | — |
| 1614. | I | 4-F | — |
| 1615. | I | 2-F | 3-F |
| 1616. | I | 2-F | 4-F |
| 1617. | I | 2-F | 5-F |
| 1618. | I | 2-F | 6-F |
| 1619. | I | 3-F | 4-F |
| 1620. | I | 3-F | 5-F |
| 1621. | I | 2-Cl | — |
| 1622. | I | 3-Cl | — |
| 1623. | I | 4-Cl | — |
| 1624. | I | 2-Cl | 3-Cl |
| 1625. | I | 2-Cl | 4-Cl |
| 1626. | I | 2-Cl | 5-Cl |
| 1627. | I | 2-Cl | 6-Cl |
| 1628. | I | 3-Cl | 4-Cl |
| 1629. | I | 3-Cl | 5-Cl |
| 1630. | I | 2-Cl | 3,4-Cl₂ |
| 1631. | I | 2-Cl | 3,5-Cl₂ |
| 1632. | I | 2-Cl | 3,6-Cl₂ |
| 1633. | I | 2-Cl | 4,5-Cl₂ |
| 1634. | I | 2-Cl | 4,6-Cl₂ |
| 1635. | I | 3-Cl | 4,5-Cl₂ |
| 1636. | I | 2-Br | — |
| 1637. | I | 3-Br | — |
| 1638. | I | 4-Br | — |
| 1639. | I | 2-Br | 3-Br |
| 1640. | I | 2-Br | 4-Br |
| 1641. | I | 2-Br | 5-Br |
| 1642. | I | 2-Br | 6-Br |
| 1643. | I | 3-Br | 4-Br |
| 1644. | I | 3-Br | 5-Br |
| 1645. | I | 2-F | 3-Cl |
| 1646. | I | 2-F | 4-Cl |
| 1647. | I | 2-F | 5-Cl |
| 1648. | I | 2-F | 3-Br |
| 1649. | I | 2-F | 4-Br |
| 1650. | I | 2-F | 5-Br |
| 1651. | I | 2-Cl | 3-Br |
| 1652. | I | 2-Cl | 4-Br |
| 1653. | I | 2-Cl | 5-Br |
| 1654. | I | 3-F | 4-Cl |
| 1655. | I | 3-F | 5-Cl |
| 1656. | I | 3-F | 6-Cl |
| 1657. | I | 3-F | 4-Br |
| 1658. | I | 3-F | 5-Br |
| 1659. | I | 3-F | 6-Br |
| 1660. | I | 3-Cl | 4-Br |
| 1661. | I | 3-Cl | 5-Br |
| 1662. | I | 3-Cl | 6-Br |
| 1663. | I | 4-F | 5-Cl |
| 1664. | I | 4-F | 6-Cl |
| 1665. | I | 4-F | 5-Br |
| 1666. | I | 4-F | 6-Br |
| 1667. | I | 4-Cl | 5-Br |
| 1668. | I | 5-F | 6-Cl |
| 1669. | I | 5-Fr | 6-Br |
| 1670. | I | 5-Cl | 6-Br |
| 1671. | I | 3-Br | 4-Cl, 5-Br |
| 1672. | I | 2-CN | — |
| 1673. | I | 3-CN | — |
| 1674. | I | 4-CN | — |
| 1675. | I | 2-NO₂ | — |
| 1676. | I | 3-NO₂ | — |
| 1677. | I | 4-NO₂ | — |
| 1678. | I | 2-CH₃ | — |
| 1679. | I | 3-CH₃ | — |
| 1680. | I | 4-CH₃ | — |
| 1681. | I | 2-CH₃ | 3-CH₃ |
| 1682. | I | 2-CH₃ | 4-CH₃ |
| 1683. | I | 2-CH₃ | 5-CH₃ |
| 1684. | I | 2-CH₃ | 6-CH₃ |
| 1685. | I | 3-CH₃ | 4-CH₃ |
| 1686. | I | 3-CH₃ | 5-CH₃ |
| 1687. | I | 2-C₂H₅ | — |
| 1688. | I | 3-C₂H₅ | — |
| 1689. | I | 4-C₂H₅ | — |
| 1690. | I | 2-i-C₃H₇ | — |
| 1691. | I | 3-i-C₃H₇ | — |
| 1692. | I | 4-i-C₃H₇ | — |
| 1693. | I | 3-tert-C₄H₉ | — |
| 1694. | I | 4-tert-C₄H₉ | — |
| 1695. | I | 2-vinyl | — |
| 1696. | I | 3-vinyl | — |
| 1697. | I | 4-vinyl | — |
| 1698. | I | 2-allyl | — |
| 1699. | I | 3-allyl | — |
| 1700. | I | 4-allyl | — |
| 1701. | I | 2-C₆H₅ | — |
| 1702. | I | 3-C₆H₅ | — |
| 1703. | I | 4-C₆H₅ | — |
| 1704. | I | 3-CH₃ | 5-tert-C₄H₉ |
| 1705. | I | 2-OH | — |
| 1706. | I | 3-OH | — |
| 1707. | I | 4-OH | — |
| 1708. | I | 2-OCH₃ | — |
| 1709. | I | 3-OCH₃ | — |
| 1710. | I | 4-OCH₃ | — |
| 1711. | I | 2-OCH₃ | 3-OCH₃ |
| 1712. | I | 2-OCH₃ | 4-OCH₃ |
| 1713. | I | 2-OCH₃ | 5-OCH₃ |
| 1714. | I | 3-OCH₃ | 4-OCH₃ |
| 1715. | I | 3-OCH₃ | 5-OCH₃ |
| 1716. | I | 3-OCH₃ | 4,5-(OCH₃)₂ |
| 1717. | I | 2-OC₂H₅ | — |
| 1718. | I | 3-OC₂H₅ | — |
| 1719. | I | 4-OC₂H₅ | — |
| 1720. | I | 2-O-(n-C₃H₇) | — |
| 1721. | I | 3-O-(n-C₃H₇) | — |
| 1722. | I | 4-O-(n-C₃H₇) | — |
| 1723. | I | 2-O-(i-C₃H₇) | — |
| 1724. | I | 3-O-(i-C₃H₇) | — |
| 1725. | I | 4-O-(i-C₃H₇) | — |
| 1726. | I | 4-O-(n-C₄H₉) | — |
| 1727. | I | 3-O-(t-C₄H₉) | — |
| 1728. | I | 4-O-(t-C₄H₉) | — |
| 1729. | I | 2-O-allyl | — |
| 1730. | I | 3-O-allyl | — |
| 1731. | I | 4-O-allyl | — |
| 1732. | I | 2-CF₃ | — |
| 1733. | I | 3-CF₃ | — |
| 1734. | I | 4-CF₃ | — |
| 1735. | I | 2-acetyl | — |
| 1736. | I | 3-acetyl | — |
| 1737. | I | 4-acetyl | — |
| 1738. | I | 2-methoxycarbonyl | — |
| 1739. | I | 3-methoxycarbonyl | — |
| 1740. | I | 4-methoxycarbonyl | — |
| 1741. | I | 2-aminocarbonyl | — |
| 1742. | I | 3-aminocarbonyl | — |
| 1743. | I | 4-aminocarbonyl | — |
| 1744. | I | 2-dimethylaminocarbonyl | — |
| 1745. | I | 3-dimethylaminocarbonyl | — |
| 1746. | I | 4-dimethylaminocarbonyl | — |
| 1747. | I | 2-(N-methylaminocarbonyl) | — |
| 1748. | I | 3-(N-methylaminocarbonyl) | — |
| 1749. | I | 4-(N-methylaminocarbonyl) | — |
| 1750. | I | 2-H₂N | — |
| 1751. | I | 3-H₂N | — |
| 1752. | I | 4-H₂N | — |
| 1753. | I | 2-aminothiocarbonyl | — |
| 1754. | I | 3-aminothiocarbonyl | — |
| 1755. | I | 4-aminothiocarbonyl | — |

| | | |
|---|---|---|
| 1756. | I | 3,4-methylenedioxy |
| 1757. | I | 3,4-difluoromethylenedioxy |
| 1758. | I | 2,3-methylenedioxy |

| | | | |
|---|---|---|---|
| 1759. | I | 2-SCH₃ | — |
| 1760. | I | 3-SCH₃ | — |
| 1761. | I | 4-SCH₃ | — |
| 1762. | I | 2-SO₂CH₃ | — |
| 1763. | I | 3-SO₂CH₃ | — |
| 1764. | I | 4-SO₂CH₃ | — |
| 1765. | I | 2-OCF₃ | — |
| 1766. | I | 3-OCF₃ | — |
| 1767. | I | 4-OCF₃ | — |
| 1768. | I | 2-OCHF₂ | — |
| 1769. | I | 3-OCHF₂ | — |
| 1770. | I | 4-OCHF₂ | — |
| 1771. | I | 3-CF₃ | 4-OCF₃ |
| 1772. | I | 2-NHCH₃ | — |
| 1773. | I | 3-NHCH₃ | — |
| 1774. | I | 4-NHCH₃ | — |
| 1775. | I | 2-N(CH₃)₂ | — |
| 1776. | I | 3-N(CH₃)₂ | — |
| 1777. | I | 4-N(CH₃)₂ | — |
| 1778. | I | 2-ethoxycarbonyl | — |
| 1779. | I | 3-ethoxycarbonyl | — |
| 1780. | I | 4-ethoxycarbonyl | — |
| 1781. | I | 2-CH₂CH₂F | — |
| 1782. | I | 3-CH₂CH₂F | — |
| 1783. | I | 4-CH₂CH₂F | — |
| 1784. | I | 2-CH₂CF₃ | — |
| 1785. | I | 3-CH₂CF₃ | — |
| 1786. | I | 4-CH₂CF₃ | — |
| 1787. | I | 2-CF₂CHF₂ | — |
| 1788. | I | 3-CF₂CHF₂ | — |
| 1789. | I | 4-CF₂CHF₂ | — |
| 1790. | I | 2-CHF₂ | — |
| 1791. | I | 3-CHF₂ | — |
| 1792. | I | 4-CHF₂ | — |
| 1793. | I | 2-(1′-oxo-n-prop-1-yl) | — |
| 1794. | I | 3-(1′-oxo-n-prop-1-yl) | — |
| 1795. | I | 4-(1′-oxo-n-prop-1-yl) | — |
| 1796. | I | 2-(1′-oxoisoprop-1-yl) | — |
| 1797. | I | 3-(1′-oxoisoprop-1-yl) | — |
| 1798. | I | 4-(1′-oxoisoprop-1-yl) | — |
| 1799. | I | 3-cyclopropyl | — |
| 1800. | I | 4-cyclopropyl | — |
| 1801. | I | 4-cyclohexyl | — |
| 1802. | I | 2-methoxyiminomethyl | — |
| 1803. | I | 3-methoxyiminomethyl | — |
| 1804. | I | 4-methoxyiminomethyl | — |
| 1805. | I | 2-ethoxyiminomethyl | — |
| 1806. | I | 3-ethoxyiminomethyl | — |
| 1807. | I | 4-ethoxyiminomethyl | — |
| 1808. | I | 2-isopropyloxyiminomethyl | — |
| 1809. | I | 3-isopropyloxyiminomethyl | — |
| 1810. | I | 4-isopropyloxyiminomethyl | — |
| 1811. | I | 2-allyloxyiminomethyl | — |
| 1812. | I | 3-allyloxyiminomethyl | — |
| 1813. | I | 4-allyloxyiminomethyl | — |
| 1814. | I | 2-phenoxyiminomethyl | — |
| 1815. | I | 3-phenoxyiminomethyl | — |
| 1816. | I | 4-phenoxyiminomethyl | — |
| 1817. | I | 2-benzyloxyiminomethyl | — |
| 1818. | I | 3-benzyloxyiminomethyl | — |
| 1819. | I | 4-benzyloxyiminomethyl | — |
| 1820. | I | 2-(1-methoxyiminoeth-1-yl) | — |
| 1821. | I | 3-(1-methoxyiminoeth-1-yl) | — |
| 1822. | I | 4-(1-methoxyiminoeth-1-yl) | — |
| 1823. | I | 2-(1-isopropyloxyiminoeth-1-yl) | — |
| 1824. | I | 3-(1-isopropyloxyiminoeth-1-yl) | — |
| 1825. | I | 4-(1-isopropyloxyiminoeth-1-yl) | — |
| 1826. | I | 2-(1-allyloxyiminoeth-1-yl) | — |
| 1827. | I | 3-(1-allyloxyiminoeth-1-yl) | — |
| 1828. | I | 4-(1-allyloxyiminoeth-1-yl) | — |
| 1829. | I | 2-(1-phenoxyiminoeth-1-yl) | — |
| 1830. | I | 3-(1-phenoxyiminoeth-1-yl) | — |
| 1831. | I | 4-(1-phenoxyiminoeth-1-yl) | — |
| 1832. | I | 2-(1-benzyloxyiminoeth-1-yl) | — |
| 1833. | I | 3-(1-benzyloxyiminoeth-1-yl) | — |
| 1834. | I | 4-(1-benzyloxyiminoeth-1-yl) | — |
| 1835. | I | 2-(1-ethoxyimino-n-prop-1-yl)- | — |
| 1836. | I | 3-(1-ethoxyimino-n-prop-1-yl)- | — |
| 1837. | I | 4-(1-ethoxyimino-n-prop-1-yl) | — |
| 1838. | I | 2-(1-allyloxyimino-n-prop-1-yl) | — |
| 1839. | I | 3-(1-allyloxyimino-n-prop-1-yl) | — |
| 1840. | I | 4-(1-allyloxyimino-n-prop-1-yl) | — |

s-C₄H₉: —CH(CH₃)(C₂H₅);
i-C₄H₉: CH₂CH(CH₃)₂;
allyl: —CH₂CH═CH₂;
propargyl: —CH₂C≡CH;

Table 1:

Compounds of the formula I-A and I-B in which A is 2-chloropyridin-3-yl and R³, R⁵ and (R⁴)ₙ for each individual compound correspond in each case to one row of table A.

Table 2:

Compounds of the formula I-A and I-B in which A is 2-trifluoromethylpyridin-3-yl and R³, R⁵ and (R⁴)ₙ for each individual compound correspond in each case to one row of table A.

Table 3:

Compounds of the formula I-A and I-B in which A is 1-methyl-3-trifluoromethylpyrazol-4-yl and R³, R⁵ and (R⁴)ₙ for each individual compound correspond in each case to one row of table A.

Table 4:

Compounds of the formula I-A and I-B in which A is 1-methyl-3-difluoromethylpyrazol-4-yl and R³, R⁵ and (R⁴)ₙ for each individual compound correspond in each case to one row of table A.

Table 5:

Compounds of the formula I-A and I-B in which A is 1,3-dimethylpyrazol-4-yl and R³, R⁵ and (R⁴)ₙ for each individual compound correspond in each case to one row of table A.

Table 6:

Compounds of the formula I-A and I-B in which A is 1-methyl-3-trifluoromethyl-5-fluoropyrazol-4-yl and R³, R⁵ and (R⁴)ₙ for each individual compound correspond in each case to one row of table A.

Table 7:

Compounds of the formula I-A and I-B in which A is 1-methyl-3-difluoromethyl-5-fluoropyrazol-4-yl and R³, R⁵ and (R⁴)ₙ for each individual compound correspond in each case to one row of table A.

Table 8:

Compounds of the formula I-A and I-B in which A is 1-methyl-3-trifluoromethyl-5-chloropyrazol-4-yl and R³, R⁵ and (R⁴)ₙ for each individual compound correspond in each case to one row of table A.

Table 9:

Compounds of the formula I-A and I-B in which A is 1-methyl-3-trifluoromethylpyrrol-4-yl and R³, R⁵ and (R⁴)ₙ for each individual compound correspond in each case to one row of table A.

Table 10:

Compounds of the formula I-A and I-B in which A is 2-methyl-4-trifluoromethylthiazol-5-yl and R³, R⁵ and (R⁴)ₙ for each individual compound correspond in each case to one row of table A.

Table 11:

Compounds of the formula I-A and I-B in which A is 2-methyl-4-difluoromethylthiazol-5-yl and R³, R⁵ and (R⁴)ₙ for each individual compound correspond in each case to one row of table A.

Table 12:

Compounds of the formula I-A and I-B in which A is 2,4-dimethylthiazol-5-yl and R³, R⁵ and (R⁴)ₙ for each individual compound correspond in each case to one row of table A.

Table 13:

Compounds of the formula I-A and I-B in which A is 2-methyl-5-trifluoromethylthiazol-4-yl and R³, R⁵ and (R⁴)ₙ for each individual compound correspond in each case to one row of table A.

Table 14:

Compounds of the formula I-A and I-B in which A is 2,5-dimethylthiazol-4-yl and R³, R⁵ and (R⁴)ₙ for each individual compound correspond in each case to one row of table A.

Table 15:

Compounds of the formula I-A and I-B in which A is 2-methyl-4-trifluoromethyloxazol-5-yl and R³, R⁵ and (R⁴)ₙ for each individual compound correspond in each case to one row of table A.

Table 16:

Compounds of the formula I-A and I-B in which A is 2-trifluoromethylthiophen-3-yl and R³, R⁵ and (R⁴)ₙ for each individual compound correspond in each case to one row of table A.

Table 17:

Compounds of the formula I-A and I-B in which A is 5-methyl-2-trifluoromethylthiophen-3-yl and R³, R⁵ and (R⁴)ₙ for each individual compound correspond in each case to one row of table A.

Table 18:

Compounds of the formula I-A and I-B in which A is 3-trifluoromethylthiophen-2-yl and R³, R⁵ and (R⁴)ₙ for each individual compound correspond in each case to one row of table A.

Table 19:

Compounds of the formula I-A and I-B in which A is 2,5-dimethylfuran-3-yl and R³, R⁵ and (R⁴)ₙ for each individual compound correspond in each case to one row of table A.

Also suitable, in particular with a view to their use as fungicides and active compounds for controlling pests, are the compounds of the formulae I-C, I-D, I-E, I-F and I-G 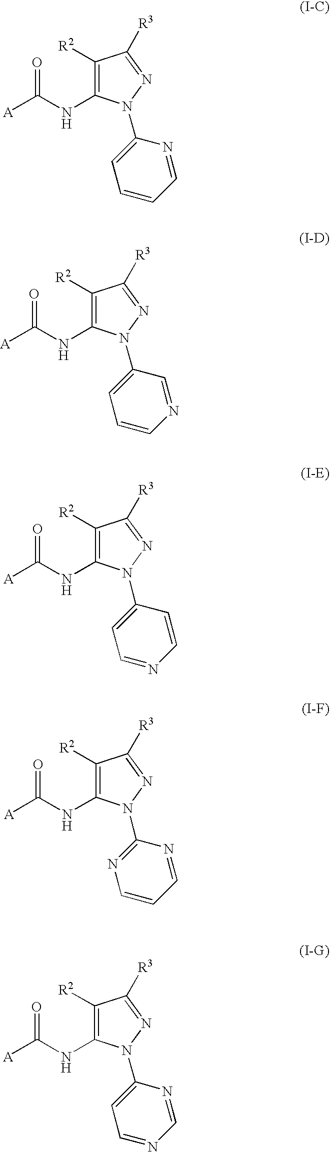
in which A, R² and R³ are as defined above, and from among these in particular those in which:
A is a radical A-1, A-2, A-3, A-4, A-5 or A-6, in particular A-1a, A-2a or A-3a, and especially a radical selected from the group consisting of 2-chloropyridin-3-yl, 2-trifluoromethylpyridin-3-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloropyrazol-4-yl, 1-methyl-3-trifluoromethylpyrrol-4-yl, 2-methyl-4-trifluoromethylthiazol-5-yl, 2-methyl-4-difluoromethylthiazol-5-yl, 2,4-dimethylthiazol-5-yl, 2-methyl-5-trifluoromethylthiazol-4-yl, 2,5-dimethyl-thiazol-4-yl, 2-methyl-4-trifluoromethyloxazol-5-yl, 2-trifluoromethylthiophen-3-yl, 5-methyl-2-trifluoromethylthiophen-3-yl, 3-trifluoromethylthiophen-2-yl and 2,5-dimethylfuran-3-yl;
R² is H or CN and
R³ is selected from the group consisting of H, methyl, trifluoromethyl, CN, NO₂ and halogen.

Examples of these are the individual compounds of the formulae I-C, I-D, I-E, I-F and I-G compiled in tables 20 to 38 below, where the variables R² and R³ each have the meanings given in one row of table B and the variable A has the meaning given in the respective tables.

TABLE B

| No. | R² | R³ |
|---|---|---|
| 1 | H | H |
| 2 | H | CN |
| 3 | H | NO₂ |
| 4 | H | CH₃ |
| 5 | H | CF₃ |
| 6 | H | Cl |
| 7 | H | Br |
| 8 | H | I |
| 9 | CN | H |
| 10 | CN | CN |
| 11 | CN | NO₂ |
| 12 | CN | CH₃ |
| 13 | CN | CF₃ |
| 14 | CN | Cl |
| 15 | CN | Br |
| 16 | CN | I |

Table 20:

Compounds of the formulae I-C, I-D, I-E, I-F and I-G in which A is 2-chloropyridin-3-yl and R² and R³ for each individual compound correspond in each case to one row of table B.

Table 21:

Compounds of the formulae I-C, I-D, I-E, I-F and I-G in which A is 2-trifluoromethyl-pyridin-3-yl and R² and R³ for each individual compound correspond in each case to one row of table B.

Table 22:

Compounds of the formulae I-C, I-D, I-E, I-F and I-G in which A is 1-methyl-3-trifluoromethylpyrazol-4-yl and R² and R³ for each individual compound correspond in each case to one row of table B.

Table 23:

Compounds of the formulae I-C, I-D, I-E, I-F and I-G in which A is 1-methyl-3-difluoromethylpyrazol-4-yl and R² and R³ for each individual compound correspond in each case to one row of table B.

Table 24:

Compounds of the formulae I-C, I-D, I-E, I-F and I-G in which A is 1,3-dimethylpyrazol-4-yl and R² and R³ for each individual compound correspond in each case to one row of table B.

Table 25:

Compounds of the formulae I-C, I-D, I-E, I-F and I-G in which A is 1-methyl-3-trifluoromethyl-5-fluoropyrazol-4-yl and R² and R³ for each individual compound correspond in each case to one row of table B.

Table 26:

Compounds of the formulae I-C, I-D, I-E, I-F and I-G in which A is 1-methyl-3-difluoromethyl-5-fluoropyrazol-4-yl and R² and R³ for each individual compound correspond in each case to one row of table B.

Table 27:

Compounds of the formulae I-C, I-D, I-E, I-F and I-G in which A is 1-methyl-3-trifluoromethyl-5-chloropyrazol-4-yl and R² and R³ for each individual compound correspond in each case to one row of table B.

Table 28:

Compounds of the formulae I-C, I-D, I-E, I-F and I-G in which A is 1-methyl-3-trifluoromethylpyrrol-4-yl and R² and R³ for each individual compound correspond in each case to one row of table B.

Table 29:

Compounds of the formulae I-C, I-D, I-E, I-F and I-G in which A is 2-methyl-4-trifluoromethylthiazol-5-yl and R² and R³ for each individual compound correspond in each case to one row of table B.

Table 30:

Compounds of the formulae I-C, I-D, I-E, I-F and I-G in which A is 2-methyl-4-difluoromethylthiazol-5-yl and R² and R³ for each individual compound correspond in each case to one row of table B.

Table 31:

Compounds of the formulae I-C, I-D, I-E, I-F and I-G in which A is 2,4-dimethylthiazol-5-yl and R² and R³ for each individual compound correspond in each case to one row of table B.

Table 32:

Compounds of the formulae I-C, I-D, I-E, I-F and I-G in which A is 2-methyl-5-trifluoromethylthiazol-4-yl and R² and R³ for each individual compound correspond in each case to one row of table B.

Table 33:

Compounds of the formulae I-C, I-D, I-E, I-F and I-G in which A is 2,5-dimethylthiazol-4-yl and R² and R³ for each individual compound correspond in each case to one row of table B.

Table 34:

Compounds of the formulae I-C, I-D, I-E, I-F and I-G in which A is 2-methyl-4-trifluoromethyloxazol-5-yl and R² and R³ for each individual compound correspond in each case to one row of table B.

Table 35:

Compounds of the formulae I-C, I-D, I-E, I-F and I-G in which A is 2-trifluoromethyl-thiophen-3-yl and R² and R³ for each individual compound correspond in each case to one row of table B.

Table 36:

Compounds of the formulae I-C, I-D, I-E, I-F and I-G in which A is 5-methyl-2-trifluoromethylthiophen-3-yl and R² and R³ for each individual compound correspond in each case to one row of table B.

Table 37:

Compounds of the formulae I-C, I-D, I-E, I-F and I-G in which A is 3-trifluoro-methylthiophen-2-yl and R² and R³ for each individual compound correspond in each case to one row of table B.

Table 38:

Compounds of the formulae I-C, I-D, I-E, I-F and I-G in which A is 2,5-dimethylfuran-3-yl and R² and R³ for each individual compound correspond in each case to one row of table B.

The compounds of the formula I according to the invention can be prepared analogously to processes known per se from the prior art, for example in accordance with scheme 1 by reacting activated (heterocyclyl)carboxylic acid derivatives II with a 5-amino-1-arylpyrazole of the formula III [Houben-Weyl: “Methoden der organ. Chemie” [Methods of organic chemistry], Georg-Thieme-Verlag, Stuttgart, New York, 1985, Volume E5, pp. 941-1045]. Activated carboxylic acid derivatives II are, for example, halides, activated esters, anhydrides, azides, e.g. chlorides, fluorides, bromides, para-nitrophenyl esters, pentafluorophenyl esters, N-hydroxysuccinimide esters, hydroxybenzotriazol-1-yl esters. In scheme 1, the radicals A, Y, R¹, R², R³, R⁴, R⁵ and n have the meanings given above, in particular the meanings mentioned as being preferred, and X is halogen, N₃, a radical derived from an activated ester, for example para-nitrophenyloxy, pentafluorophenyloxy, succinimidyloxy, benzotriazol-1-yloxy, or the radical of an aliphatic carboxylic acid, such as formyloxy, acetyloxy, etc.

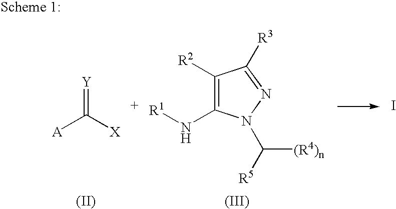

The active compounds I can also be prepared, for example, by reacting the acids IV with a 5-amino-1-arylpyrazole of the formula III in the presence of a coupling agent in accordance with scheme 2. In scheme 2, the radicals A, Y, R¹, R², R³, R⁴, R⁵, n have the meanings mentioned above and in particular the meanings mentioned as being preferred.

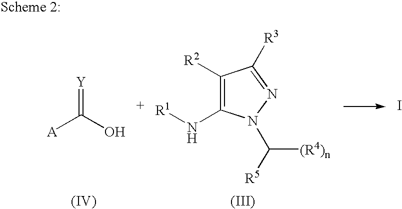

Suitable coupling agents are, for example:
coupling agents based on carbodiimide, for example N,N′-dicyclohexyl-carbodiimide [J. C. Sheehan, G. P. Hess, J. Am. Chem. Soc. 1955, 77, 1067], N-(3-dimethylaminopropyl)-N′-ethylcarbodiimide;
coupling agents which form mixed anhydrides with carbonic esters, for example 2-ethoxy-1-ethoxycarbonyl-1,2-dihydroquinoline [B. Belleau, G. Malek, J. Amer. Chem. Soc. 1968, 90, 1651.], 2-isobutyloxy-1-isobutyloxycarbonyl-1,2-dihydro-quinoline [Y. Kiso, H. Yajima, J. Chem. Soc., Chem. Commun. 1972, 942.];
phosphonium-based coupling agents, for example (benzotriazol-1-yloxy)-tris(dimethylamino)phosphonium hexafluorophosphate [B. Castro, J. R. Domoy, G. Evin, C. Selve, Tetrahedron Lett. 1975, 14, 1219.], (benzotriazol-1-yl-oxy)tripyrrolidinophosphonium hexafluorophosphate [J. Coste et. al., Tetrahedron Lett. 1990, 31, 205.];
uronium-based coupling agents or coupling agents having a guanidinium N-oxide structure, for example N,N,N′,N′-tetramethyl-O-(1H-benzotriazol-1-yl)-uronium hexafluorophosphate [R. Knorr, A. Trzeciak, W. Bannwarth, D. Gillessen, Tetrahedron Lett. 1989, 30, 1927.], N,N,N′,N′-tetramethyl-O-(benzotriazol-1-yl)uronium tetrafluoroborate, (benzotriazol-1-yloxy)-dipiperidinocarbenium hexafluorophosphate [S. Chen, J. Xu, Tetrahedron Lett. 1992, 33, 647.];
coupling agents which form acid chlorides, for example bis(2-oxo-3-oxazolidinyl)-phosphinic chloride [J. Diago-Mesequer, Synthesis 1980, 547.].

Compounds of the formula I in which R¹ is a radical different from hydrogen, for example optionally halogen-substituted alkyl or optionally substituted cycloalkyl, can also be prepared by alkylation of the amides I (where R¹ is hydrogen and which are obtainable according to scheme 1 or 2) using suitable alkylating agents in the presence of bases, see scheme 3.

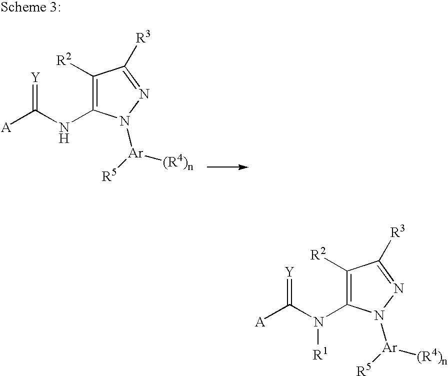

The required reaction conditions are known to the person skilled in the art, for example from J. March, Advanced Organic Synthesis.

The (heterocyclyl)carboxylic acids IV can be prepared by methods known from the literature, and they can be used to prepare, by methods known from the literature, the (heterocyclyl)carboxylic acid derivatives II [for example EP 0589313, EP 915868, U.S. Pat. No. 4,877,441].

The 5-amino-1-arylpyrazoles of the formula III are known or can be prepared, for example, in accordance with the process shown in scheme 4. In scheme 4, the radicals R², R³, R⁴, R⁵ and n have the meanings given above and in particular the meanings given as being preferred. The 1-arylhydrazines of the formula V and the 2,3-dichloropropionitriles of the formula VI are known from the literature or can be prepared by methods known from the literature.

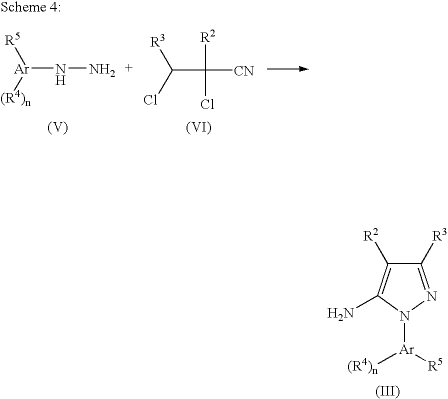

The reaction of V with VI can be carried out analogously to methods known from the literature, as described, for example, by Dorn et al., J. Prakt. Chem. 321, (1979), p. 93.

Instead of 2,3-dichloropropionitriles, it is also possible to use 2-chloroacrylonitriles of the formula VII, which gives pyrazoles of the formula III in which R² is hydrogen. The reaction of V with VII can be carried out analogously to methods known from the literature, as described, for example, by P. Schmidt et al., Helv. Chim. Acta, 41 (1958), p. 306, Dorn et al., Org. Synth. 48 (1968), p. 8; Fabron et al., J. Fluorine Chem., 37 (1987), p. 371.

Instead of 2,3-dichloropropionitriles, it is also possible to use 3,3-dicyanoacrylic esters of the formula VIII, which gives pyrazoles of the formula III in which R³ is CN. The reaction of V with VIII can be carried out analogously to methods known from the literature, as described, for example, by Ferrooni et al., Arzneim.-Forsch. 40 (1990), pp. 1328-1331; N. P. Peet et al., J. Heterocycl. Chem., 20 (1983), p. 511.

Instead of 2,3-dichloropropionitriles, it is also possible to use 3-aminopropionitriles of the formula IX, which gives pyrazoles of the formula III. The reaction of V with IX can be carried out analogously to methods known from the literature, as described, for example, by A. Ganesan et al., J. Heterocycl. Chem., 15 (1978), p. 715.

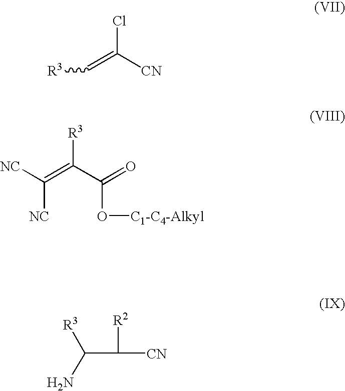

The compounds I are suitable for use as fungicides. They are distinguished by an outstanding effectiveness against a broad spectrum of phytopathogenic fungi, especially from the classes of the Ascomycetes, Deuteromycetes, Phycomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Botrytis cinerea (gray mold) on strawberries, vegetables, ornamental plants and grapevines,
Cercospora arachidicola on groundnuts,
Erysiphe cichoracearum and Sphaerotheca fuliginea on cucurbits,
Erysiphe graminis (powdery mildew) on cereals,
Fusarium and Verticillium species on various plants,
Helminthosporium species on cereals,
Mycosphaerella species on bananas and groundnuts,
Phakopsora species on soybean,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria nodorum on wheat,
Sphaerotheca fuliginea (mildew of cucumber) on cucumbers,
Uncinula necator on grapevines,
Ustilago species on cereals and sugar cane,
Venturia species (scab) on apples and pears,
Septoria tritici,
Pyrenophora species,
Leptosphaeria nodorum,
Rhynchosporium species and
Typhula species.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should in any case guarantee a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic ores (e.g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutylnaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ethers, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Petroleum fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, combinations for broadcasting and dusts can be prepared by mixing or mutually grinding the active substances with a solid carrier.

Granules, e.g. coated granules, impregnated granules and homogeneous granules, can be prepared by binding the active compounds to solid carriers. Solid carriers are, e.g., mineral earths, such as silica gel, silicic acids, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for formulations are:
I. 5 parts by weight of a compound according to the invention are intimately mixed with 95 parts by weight of finely divided kaolin. In this way, a dust comprising 5% by weight of the active compound is obtained.
II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.
III. 10 parts by weight of a compound according to the invention are dissolved in a mixture consisting of 90 parts by weight of xylene, 6 parts by weight of the addition product of 8 to 10 mol of ethylene oxide with 1 mol of the N-mono-ethanolamide of oleic acid, 2 parts by weight of the calcium salt of dodecyl-benzenesulfonic acid and 2 parts by weight of the addition product of 40 mol of ethylene oxide with 1 mol of castor oil (active compound content 9% by weight).
IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).
V. 80 parts by weight of a compound according to the invention are intimately mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).
VI. 90 parts by weight of a compound according to the invention are mixed with 10 parts by weight of N-methyl-α-pyrrolidone and a solution is obtained which is suitable for use in the form of very small drops (active compound content 90% by weight).
VII. 20 parts by weight of a compound according to the invention are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. By running the solution into 100 000 parts by weight of water and finely dispersing it therein, an aqueous dispersion is obtained comprising 0.02% by weight of the active compound.
VIII. 20 parts by weight of a compound according to the invention are intimately mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.
IX. 10 parts by weight of the compound according to the invention are dissolved in 63 parts by weight of cyclohexanone, 27 parts by weight of dispersing agent (for example a mixture of 50 parts by weight of the adduct of 7 mol of ethylene oxide to 1 mol of isooctylphenol and 50 parts by weight of the adduct of 40 mol of ethylene oxide to 1 mol of castor oil). The stock solution is then diluted to the desired concentration, for example a concentration in the range from 1 to 100 ppm, by distribution in water.

The active compounds can be used as such, in the form of their formulations or of the application forms prepared therefrom, e.g. in the form of directly sprayable solutions, powders, suspensions or dispersions, emulsions, oil dispersions, pastes, dusts, compositions for broadcasting or granules, by spraying, atomizing, dusting, broad-casting or watering. The application forms depend entirely on the intended uses; they should in any case guarantee the finest possible dispersion of the active compounds according to the invention.

Aqueous application forms can be prepared from emulsion concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-to-use preparations can be varied within relatively wide ranges. In general, they are between 0.0001 and 10%. Often even small amounts of active compound I are sufficient in the ready-to-use preparation, for example 2 to 200 ppm. Ready-to-use preparations with concentrations of active compound in the range from 0.01 to 1% are also preferred.

The active compounds can also be used with great success in the ultra low volume (ULV) process, it being possible to apply formulations with more than 95% by weight of active compound or even the active compound without additives.

Oils of various types, herbicides, fungicides, other pesticides and bactericides can be added to the active compounds, if need be also not until immediately before use (tank mix). These agents can be added to the compositions according to the invention in a weight ratio of 1:10 to 10:1.

The compositions according to the invention can, in the application form as fungicides, also be present together with other active compounds, e.g. with herbicides, insecticides, growth regulators, fungicides or also with fertilizers. On mixing the compounds I or the compositions comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but does not limit them:
sulfur, dithiocarbamates and their derivatives, such as iron(III) dimethyldithiocarbamate, zinc dimethyldithiocarbamate, zinc ethylenebisdithiocarbamate, manganese ethylenebisdithiocarbamate, manganese zinc ethylenediaminebis-dithiocarbamate, tetramethylthiuram disulfide, ammonia complex of zinc (N,N′-ethylenebisdithiocarbamate), ammonia complex of zinc (N,N′-propylene-bisdithiocarbamate), zinc (N,N′-propylenebisdithiocarbamate) or N,N′-poly-propylenebis(thiocarbamoyl)disulfide;
nitro derivatives, such as dinitro(1-methylheptyl)phenyl crotonate, 2-sec-butyl-4,6-dinitrophenyl 3,3-dimethylacrylate, 2-sec-butyl-4,6-dinitrophenyl isopropyl carbonate or diisopropyl 5-nitroisophthalate;
heterocyclic substances, such as 2-heptadecyl-2-imidazoline acetate, 2,4-dichloro-6-(o-chloroanilino)-s-triazine, O,O-diethyl phthalimidophosphono-thioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-(methoxycarbonylamino)benzimidazole, 2-(2-furyl)benzimidazole, 2-(4-thiazolyl)benzimidazole, N-(1,1,2,2-tetrachloroethylthio)tetrahydrophthalimide, N-(trichloromethylthio)tetrahydrophthalimide or N-(trichloromethylthio)phthalimide,
N-dichlorofluoromethylthio-N′,N′-dimethyl-N-phenylsulfdionic acid diamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, 2-thiopyridine 1-oxide, 8-hydroxyquinoline or its copper salt, 2,3-dihydro-5-carboxanilido-6-methyl-1,4-oxathiin, 2,3-dihydro-5-carboxanilido-6-methyl-1,4-oxathiin 4,4-dioxide, 2-methyl-5,6-dihydro-4H-pyran-3-carboxanilide, 2-methylfuran-3-carboxanilide, 2,5-dimethylfuran-3-carboxanilide, 2,4,5-trimethyl-furan-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine 2,2,2-trichloroethyl acetal, piperazine-1,4-diylbis-1-(2,2,2-trichloroethyl)formamide, 1-(3,4-dichloroanilino)-1-formyl-amino-2,2,2-trichloroethane, 2,6-dimethyl-N-tridecylmorpholine or its salts, 2,6-dimethyl-N-cyclododecylmorpholine or its salts, N-[3-(p-(tert-butyl)phenyl)-2-methylpropyl]-cis-2,6-dimethylmorpholine, N-[3-(p-(tert-butyl)phenyl)-2-methyl-propyl]piperidine, 1-[2-(2,4-dichlorophenyl)-4-ethyl-1,3-dioxolan-2-ylethyl]-1H-1,2,4-triazole, 1-[2-(2,4-dichlorophenyl)-4-(n-propyl)-1,3-dioxolan-2-ylethyl]-1H-1,2,4-triazole, N-(n-propyl)-N-(2,4,6-trichlorophenoxyethyl)-N′-imidazolylurea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)oxiran-2-ylmethyl]-1H-1,2,4-triazole, α-(2-chlorophenyl)-α-(4-chlorophenyl)-5-pyrimidine methanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene or 1,2-bis(3-methoxycarbonyl-2-thioureido)benzene,
strobilurins, such as methyl E-methoxyimino[α-(o-tolyloxy)-o-tolyl]acetate, methyl E-2-{2-[6-(2-cyanophenoxy)pyrimidin-4-yloxy]phenyl}-3-methoxyacrylate, methyl E-methoxyimino-[α-(2-phenoxyphenyl)]acetamide, methyl E-methoxyimino-[α-(2,5-dimethylphenoxy)-o-tolyl]acetamide,
anilinopyrimidines, such as N-(4,6-dimethylpyrimidin-2-yl)aniline, N-[4-methyl-6-(1-propynyl)pyrimidin-2-yl]aniline or N-[4-methyl-6-cyclopropylpyrimidin-2-yl]-aniline,
phenylpyrroles, such as 4-(2,2-difluoro-1,3-benzodioxol-4-yl)pyrrole-3-carbonitrile,
cinnamamides, such as 3-(4-chlorophenyl)-3-(3,4-dimethoxyphenyl)acryloyl-morpholine,
and various fungicides, such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, N-(2,6-dimethylphenyl)-N-(2′-methoxy-acetyl)-DL-alanine methyl ester, N-(2,6-dimethylphenyl)-N-chloroacetyl-D,L-2-aminobutyrolactone, N-(2,6-dimethylphenyl)-N-(phenylacetyl)-DL-alanine methyl ester, 5-methyl-5-vinyl-3-(3,5-dichlorophenyl)-2,4-dioxo-1,3-oxazolidine, 3-(3,5-dichlorophenyl)-5-methyl-5-methoxymethyl-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-N-(ethylaminocarbonyl)-2-[methoxyimino]acetamide, 1-[2-(2,4-dichlorophenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### PREPARATION EXAMPLES

### Example 1

### 2-Chloro-N-(2-(2,4-dichlorophenyl)-2H-pyrazol-3-yl)nicotinamide

### 1.1 3-Amino-2-(2,4-dichlorophenyl)-2H-pyrazole

A solution of 6.2 g of 2,4-dichlorophenylhydrazine, 0.01 g of Titriplex III and 9.4 g of 2-chloroacrylonitrile in 50 ml of methanol was stirred at 70° C. for 6 h. 5.5 g of concentrated sulfuric acid was then added, and the mixture was stirred for 1 h and subsequently concentrated under reduced pressure. 50 ml of water and 11.1 g of sodium carbonate were added to the residue, and the mixture was extracted twice with methyl tert-butyl ether. The combined organic phases were washed with water and aqueous sodium chloride solution, dried over sodium sulfate, concentrated and subsequently subjected to chromatographic work-up. This gave 5.8 g of the title compound.

### 1.2 2-Chloro-N-(2-(2,4-dichlorophenyl)-2H-pyrazol-3-yl)nicotinamide

A solution of 0.39 g of 2-chloronicotinoyl chloride, 0.45 g of triethylamine and 0.50 g of 3-amino-2-(2,4-dichlorophenyl)-2H-pyrazole in 10 ml of dichloromethane was stirred at 41° C. for 6 hours. The reaction mixture was washed with 5% strength aqueous hydrochloric acid, diluted with aqueous sodium hydroxide solution and water, dried over sodium sulfate, filtered and then evaporated to dryness. Chromatographic purification gave 0.28 g of the title compound in the form of colorless crystals of melting point 162-164° C.

The compounds of the formula I-A listed in table C were prepared in an analogous manner.

TABLE C

| | |
|---|---|
| | (I-A) |
| 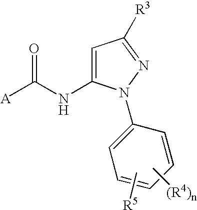 | |

| | | | | | m.p.[° C.]¹⁾; | Spectroscopic |
|---|---|---|---|---|---|---|
| No. | A | R³ | (R⁴)ₙ | R⁵ | consistency | data²⁾ |
| 1 | 2-chloropyridin-3-yl | H | 2-Cl | 4-Cl | 162-164 | — |
| 2 | 2-chloropyridin-3-yl | CH₃ | — | 4-CH₃ | 167-169 | — |
| 3 | 2-chloropyridin-3-yl | CF₃ | — | 4-I | 181-184 | — |
| 4 | 2-chloropyridin-3-yl | CF₃ | — | 4-F | 177-179 | — |
| 5 | 2-chloropyridin-3-yl | CF₃ | — | 4-Br | 160-162 | — |
| 6 | 2-chloropyridin-3-yl | CF₃ | — | 4-Cl | 170-173 | — |
| 7 | 2-methyl-4-trifluoro- | CF₃ | — | 4-F | 150-154 | — |
| | methylthiazol-5-yl | | | | | |
| 8 | 2-methyl-4-trifluoro- | CF₃ | — | 4-I | 180-183 | — |
| | methylthiazol-5-yl | | | | | |
| 9 | 2-methyl-4-trifluoro- | CF₃ | — | 4-Br | 160-163 | — |
| | methylthiazol-5-yl | | | | | |
| 10 | 1,3-dimethylpyrazol- | CF₃ | — | H | 224-226 | — |
| | 4-yl | | | | | |
| 11 | 1,3-dimethylpyrazol- | CF₃ | — | 4-F | 210-212 | — |
| | 4-yl | | | | | |
| 12 | 1,3-dimethylpyrazol- | CF₃ | — | 4-Cl | 203-205 | — |
| | 4-yl | | | | | |
| 13 | 1,3-dimethylpyrazol- | CF₃ | — | 4-Br | 200-203 | — |
| | 4-yl | | | | | |
| 14 | 1,3-dimethylpyrazol- | CF₃ | — | 4-I | 203-205 | — |
| | 4-yl | | | | | |
| 15 | 3-methylthiophen-2- | CF₃ | — | H | 142-143 | — |
| | yl | | | | | |
| 16 | 3-methylthiophen-2- | CF₃ | — | 4-F | 158-160 | — |
| | yl | | | | | |
| 17 | 3-methylthiophen-2- | CF₃ | — | 4-Cl | 161-163 | — |
| | yl | | | | | |
| 18 | 3-methylthiophen-2- | CF₃ | — | 4-Br | 182-184 | — |
| | yl | | | | | |
| 19 | 3-methylthiophen-2- | CF₃ | — | 4-I | 209-211 | — |
| | yl | | | | | |
| 20 | 1-methyl-3-trifluoro- | CF₃ | — | H | 210-212 | — |
| | methylpyrazol-4-yl | | | | | |
| 21 | 1-methyl-3-trifluoro- | CF₃ | — | 4-F | 178-180 | — |
| | methylpyrazol-4-yl | | | | | |
| 22 | 1-methyl-3-trifluoro- | CF₃ | — | 4-Cl | 180-184 | — |
| | methylpyrazol-4-yl | | | | | |
| 23 | 1-methyl-3-trifluoro- | CF₃ | — | 4-Br | 196-200 | — |
| | methylpyrazol-4-yl | | | | | |
| 24 | 1-methyl-3-trifluoro- | CF₃ | — | 4-I | 209-212 | — |
| | methylpyrazol-4-yl | | | | | |
| 25 | 4-methylthiazol-5-yl | CF₃ | — | H | 148-150 | — |
| 26 | 4-methylthiazol-5-yl | CF₃ | — | 4-F | 158-161 | — |
| 27 | 4-methylthiazol-5-yl | CF₃ | — | 4-Cl | 145-147 | — |
| 28 | 4-methylthiazol-5-yl | CF₃ | — | 4-Br | 163-166 | — |
| 29 | 4-methylthiazol-5-yl | CF₃ | — | 4-I | 175-179 | — |
| 30 | 2-chloropyridin-3-yl | CF₃ | — | H | 174-176 | — |
| 31 | 2-chloropyridin-3-yl | H | — | 4-CH₃ | oil | — |
| 32 | 1-methyl-3-trifluoro- | H | — | 4-CH₃ | oil | — |
| | methylpyrazol-4-yl | | | | | |
| 33 | 2-methyl-4- | H | — | 4-CH₃ | 153-156 | — |
| | trifluoromethyl- | | | | | |
| | thiazol-5-yl | | | | | |
| 34 | 2-chloropyridin-3-yl | H | — | 4-Cl | oil | ¹H-NMR |
| | | | | | | (DMSO-D₆): δ |
| | | | | | | [ppm]: 6.61-6.63 |
| | | | | | | (m, 1H), 7.77-8.09 |
| | | | | | | (m, 7H), |
| | | | | | | 8.55-8.58(m, |
| | | | | | | 1H), 10.80(br s, |
| | | | | | | 1H) |
| 35 | 1-methyl-3-trifluoro- | H | — | 4-Cl | oil | ¹H-NMR |
| | methylpyrazol-4-yl | | | | | (CDCl₃): δ |
| | | | | | | [ppm]: 3.95(s, |
| | | | | | | 3H), 6.75-6.79 |
| | | | | | | (m, 1H), 7.50-7.79 |
| | | | | | | (m, 6H), |
| | | | | | | 8.39(br s, 1H) |
| 36 | 2-methyl-4-trifluoro- | H | — | 4-Cl | oil | ¹H-NMR |
| | methylthiazol-5-yl | | | | | (CDCl₃): δ |
| | | | | | | [ppm]: 2.62(s, |
| | | | | | | 3H), 6.43(br s, |
| | | | | | | 1H), 7.15-7.22 |
| | | | | | | (m, 5H), 8.92(br |
| | | | | | | s, 1H) |
| 37 | 1-methyl-3-trifluoro- | H | 2-Cl | 4-Cl | 158-163 | — |
| | methylpyrazol-4-yl | | | | | |
| 38 | 2-methyl-4-trifluoro- | H | 2-Cl | 4-Cl | 178-179 | — |
| | methylthiazol-5-yl | | | | | |
| 39 | 2-chloropyridin-3-yl | H | — | 4-Br | 162-165 | — |
| 40 | 1-methyl-3-trifluoro- | H | — | 4-Br | 142-144 | — |
| | methylpyrazol-4-yl | | | | | |
| 41 | 2-methyl-4-trifluoro- | H | — | 4-Br | 155-157 | — |
| | methylthiazol-5-yl | | | | | |
| 42 | 2-chloropyridin-3-yl | H | — | 4-phenyl | 150-152 | — |
| 43 | 1-methyl-3-trifluoro- | H | — | 4-phenyl | oil | ¹H-NMR |
| | methylpyrazol-4-yl | | | | | (CDCl₃): δ |
| | | | | | | [ppm]: 3.88(s, |
| | | | | | | 3H), 6.48(s, |
| | | | | | | 1H), 7.30-7.70 |
| | | | | | | (m, 10H), 8.15 |
| | | | | | | (br s, 1H) |
| 44 | 2-methyl-4-trifluoro- | H | — | 4-phenyl | oil | ¹H-NMR |
| | methylthiazol-5-yl | | | | | (CDCl₃): δ |
| | | | | | | [ppm]: 2.63(s, |
| | | | | | | 3H), 6.72(br s, |
| | | | | | | 1H), 7.35-7.78 |
| | | | | | | (m, 10H), 8.32 |
| | | | | | | (br s, 1H) |
| 45 | 2-chloropyridin-3-yl | H | — | 4-(4-Cl- | 169-171 | — |
| | | | | phenyl) | | |
| 46 | 1-methyl-3-trifluoro- | H | — | 4-(4-Cl- | 203-205 | — |
| | methylpyrazol-4-yl | | | phenyl) | | |

¹⁾m.p. = melting point
²⁾s: singlet; m: multiplet; br.s. broad singlet

### USE EXAMPLES

For the use examples 1 to 3, the active compounds were prepared as a stock solution comprising 0.25% by weight of active compound in acetone or dimethyl sulfoxide (DMSO). 1% by weight of the emulsifier Uniperol® EL (wetting agent having emulsifying and dispersant action based on ethoxylated alkylphenols) was added to this solution, and the mixture was diluted with water to the desired concentration.

For the use examples 4 to 7, the active compounds were prepared as a stock solution comprising 25 mg of active compound which was made up with a mixture of acetone and/or DMSO and the emulsifier Uniperol® EL in a volume ratio of solvent/emulsifier of 99:1 to 10 ml. The mixture was then made up to 100 ml with water. This stock solution was diluted to the active compound concentration stated below using the solvent/emulsifier/water mixture described.

### Use Example 1

### Activity Against Peronospora of Grapevines caused by Plasmopara viticola

Leaves of potted grapevines were sprayed to runoff point with an aqueous suspension having the concentration of active compounds stated below. The next day, the undersides of the leaves were inoculated with an aqueous sporangia suspension of Plasmopara viticola. The grapevines were then initially placed in a water vapor-saturated chamber at 24° C. for 48 hours and then in a greenhouse at temperatures between 20 and 30° C. for 5 days. After this time, the plants were again placed in a humid chamber for 16 hours to promote sporangiophore eruption. The extent of the development of the infection on the undersides of the leaves was then determined visually.

In this test, the plants which had been treated with 250 ppm of the active compound from example 5, example 6, example 7, example 9, example 10, example 11, example 19, example 21, example 27, example 28 or example 29 of table C showed an infection of at most 10%, the plants which had been treated with 250 ppm of the active compound from example 18 of table C showed an infection of at most 20%, whereas the untreated plants were 80% infected.

### Use Example 2

### Curative Activity Against Brown Rust of Wheat Caused by Puccinia recondita

Leaves of potted wheat seedlings of the cultivar “Kanzler” were inoculated with spores of brown rust (Puccinia recondite). The pots were then placed in a chamber with high atmospheric humidity (90 to 95%) at 20 to 22° C. for 24 hours. During this time, the spores germinated and the germ tubes penetrated into the leaf tissue. The next day, the infected plants were sprayed to runoff point with an aqueous suspension having the concentration of active compounds stated below. The suspensions or emulsions were prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

In this test, the plants which had been treated with 250 ppm of the active compound from example 3, example 4, example 5, example 6, example 21, example 23 or example 30 of table C showed an infection of at most 10%, whereas the untreated plants were 90% infected.

### Use Example 3

### Activity Against Early Blight of Tomato Caused by Alternaria solani

Leaves of potted plants of the cultivar “Goldene Königin” were sprayed to runoff point with an aqueous suspension having the concentration of active compound stated below. The next day, the leaves were infected with an aqueous spore suspension of Alternaria solani in a 2% strength biomalt solution having a density of 0.17×10⁶ spores/ml. The plants were then placed in a water vapor-saturated chamber at temperatures between 20 and 22° C. After 5 days, the disease on the untreated but infected control plants had developed to such an extent that the infection could be determined visually in %.

In this test, the plants which had been treated with 250 ppm of the active compound from example 42, example 44 or example 46 of table C showed an infection of at most 5%, the plants which had been treated with 250 ppm of the active compound from example 41 of table C showed an infection of at most 20%, whereas the untreated plants were 90% infected. Plants which had been treated with 300 ppm of the active compound from example 34 or from example 36 of table C showed an infection of at most 20%, whereas the untreated plants were 80% infected.

### Use Example 4

### Activity Against Net Blotch of Barley Caused by Pyrenophora teres, 1 Day Protective Application

Leaves of potted barley seedlings were sprayed to runoff point with an aqueous suspension having the concentration of active compounds stated below. 24 hours after the spray coating had dried on, the plants were inoculated with an aqueous spore suspension of Pyrenophora [syn. Drechslera] teres, the net blotch pathogen. The test plants were then placed in a greenhouse at temperatures between 20 and 24° C. and at 95 to 100% relative atmospheric humidity. After 6 days, the extent of the development of the disease was determined visually by the infected leaf area in %.

In this test, the plants which had been treated with 250 ppm of the active compound from example 3, example 4, example 6, example 15 or example 35 of table C showed an infection of at most 10%, whereas the untreated plants were 90% affected.

### Use Example 5

### Activity Against Peronospora of Grapevines Caused by Plasmopara viticola, 7 Day Protective Application

Leaves of potted grapevines were sprayed to runoff point with an aqueous suspension having the concentration of active compound stated below. To be able to assess the persistency of the substances, the plants were, after the spray coating had dried on, placed in a greenhouse for 7 days. The undersides of the leaves were then inoculated with an aqueous sporangia suspension of Plasmopara viticola. The grapevines were then placed initially for 48 hours in a water vapor-saturated chamber at 24° C. and then for 5 days in the greenhouse at temperatures between 20 and 30° C. After this time, the plants were again placed in a humid chamber for 16 hours, to accelerate sporangiophore eruption. The extent of the development of the infection on the undersides of the leaves was then determined visually.

In this test, the plants which had been infected with 250 ppm of the active compound from example 8, example 9 or example 11 of table C showed an infection of at most 10%, the plants which had been treated with 250 ppm of the active compound from example 10 of table C showed an infection of at most 20%, whereas the untreated plants were 50% infected.

### Use Example 6

### Activity Against Gray Mold on Bell Pepper Leaves Caused by Botrytis cinerea, Protective Application

Bell pepper leaves of the cultivar “Neusiedler Ideal Elite” were, after 2 to 3 leaves were well developed, sprayed to runoff point with an aqueous suspension having the concentration of active compound stated below. The next day, the treated plants were inoculated with an aqueous spore suspension of Botrytis cinerea in a 2% strength aqueous biomold solution having a density of 1.7×10⁶ spores/ml. The plants were then placed in a dark acclimatized chamber at temperatures between 22 and 24° C. and high atmospheric humidity. After 5 days, the extent of the fungal infection could be determined visually by the infection of the leaf area.

In this test, the plants which had been treated with 250 ppm of the active compound from example 42 or example 44 of table C showed an infection of at most 5%, the plants which had been treated with 63 ppm of the active compound from example 43 of table C showed an infection of at most 10%, whereas the untreated plants were 90% affected.

### Use Example 7

### Activity Against Mildew of Wheat Caused by Erysiphe [syn. Blumeria] graminis forma specialis. tritici

Leaves of potted wheat seedlings of the cultivar “Kanzler” were sprayed to runoff point with an aqueous suspension having the concentration of active compound stated below. 24 h after the spray coating had dried on, the leaves were dusted with spores of mildew of wheat (Erysiphe [syn. Blumeria] graminis forma specialis. tritici). The plants were then placed in a greenhouse at temperatures between 20 and 24° C. and 60 to 90% relative atmospheric humidity. After 7 days, the extent of the fungal infection was determined visually by the infection of the leaf area.

In this test, the plants which had been treated with 250 ppm of the active compound from example 31 of table C showed an infection of at most 15%, whereas the untreated plants were 70% infected.

1. A method for controlling harmful fungi, which comprises treating the harmful fungi, their habitat or the plants, areas, materials or spaces to be kept free from them with a fungicidally effective amount of at least one (hetero)cyclylcarboxamide of the formula I, 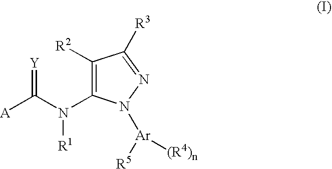
in which the variables are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries 1, 2 or 3 radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R², R³ independently of one another are hydrogen, halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁴ is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁵ is hydrogen, halogen, nitro, CN, OH, C₁-C₆-alkyl, C₃-C₆-cycloalkyl, C₂-C₄-alkenyl, C₂-C₄-alkynyl, C₁-C₄-alkoxy, C₂-C₄-alkenyloxy, C₂-C₄-alkynyloxy, C₁-C₄-alkoxy-C₁-C₄-alkoxy, C₁-C₆-haloalkyl, C₃-C₆-halocycloalkyl, C₂-C₄-haloalkenyl, C₂-C₄-haloalkynyl, C₁-C₄-haloalkoxy; C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, —(CR⁶)═NOR⁷, —C(O)R⁸, NR⁹R¹⁰, —C(O)NR⁹R¹⁰, —C(S)NR⁹R¹⁰, phenyl or phenyl-C₁-C₄-alkyl, where the phenyl ring in the two last-mentioned radicals may optionally have 1, 2, 3 or 4 of the radicals mentioned under R⁴, where
R⁶ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b};
R⁷ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl and the phenyl group in phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl and phenyl-C₂-C₄-haloalkynyl may be unsubstituted or may carry one, two or three radicals R^{b};
R⁸ is hydrogen, OH, C₁-C₄-alkyl, C₁-C₄-alkoxy, C₂-C₄-alkenyl, C₂-C₄-alkynyl, C₂-C₄-alkenyloxy, C₂-C₄-alkynyloxy, C₁-C₄-alkoxy-C₁-C₄-alkoxy, where some or all of the hydrogen atoms in the 7 last-mentioned groups may be replaced by halogen; and
R⁹, R¹⁰ independently of one another are hydrogen, C₁-C₄-alkyl, C₂-C₄-alkenyl, C₂-C₄-alkynyl, where some or all of the hydrogen atoms in these groups may be replaced by halogen;
and where two radicals R⁴ and R⁵ attached to adjacent carbon atoms may also be an alkylene chain having 3 to 5 members in which 1 or 2 non-adjacent CH₂ groups may also be replaced by oxygen or sulfur and in which some or all hydrogens may be replaced by halogen;
Ar is phenyl, naphthyl or a 5- or 6-membered heteroaromatic radical having 1, 2 or 3 heteroatoms selected from the group consisting of N, O and S as ring members, which radical may, if appropriate, also carry a fused-on benzene ring,
n is 0, 1, 2, 3 or 4;
and/or at least one agriculturally useful salt thereof.

2. The method according to claim 1 in which A in formula I is a radical of the formulae 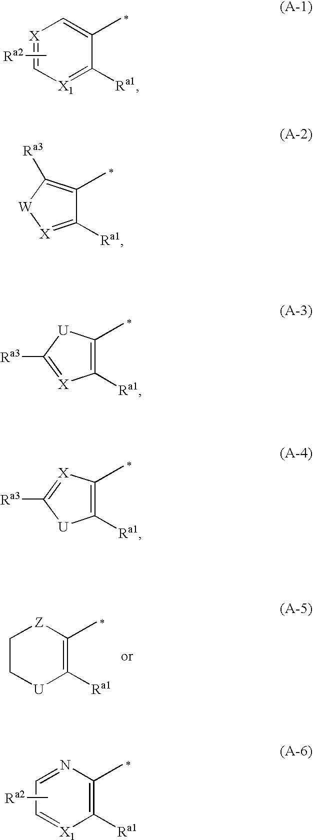
where * is the point of attachment to C(═Y) and the variables are as defined below:
X, X₁ are each independently of one another N or CR^{c}, where R^{c} is H or has one of the meanings mentioned for R^{b};
W is S or N—R^{a4}, where R^{a4} is hydrogen, C₁-C₄-alkyl, C₁-C₄-haloalkyl, C₁-C₄-alkoxy, C₁-C₄-haloalkoxy or phenyl which may be unsubstituted or may carry 1, 2 or 3 radicals R^{b};
U is oxygen or sulfur;
Z is S, S(═O), S(═O)₂ or CH₂,
R^{a1} is hydrogen, C₁-C₄-alkyl, C₁-C₄-alkoxy, C₁-C₄-haloalkyl, C₁-C₄-haloalkoxy or halogen;
R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen.

3. The method according to claim 2 in which R^{a1} is hydrogen, halogen, C₁-C₂-alkyl, C₁-C₂-alkoxy or C₁-C₂-fluoroalkyl.

4. The method according to claim 2 in which A in formula I is a radical of the formula A-1a, A-2a or A-3a, 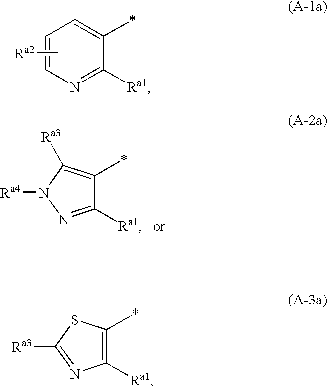
in which R^{a1}, R^{a2}, R^{a3} and R^{a4} are as defined in claim 2 or 3.

5. The method according to claim 4 in which A in formula I is a radical A-1a where R^{a1}=halogen and R^{a2}=hydrogen, or is a radical A-2a where R^{a1}=C₁-C₂-fluoroalkyl, R^{a3}=hydrogen and R^{a4}=C₁-C₄-alkyl or is a radical A-3a where R^{a1}=C₁-C₂-fluoroalkyl and R^{a3}=C₁-C₄-alkyl.

6. The method according to claim 1 in which R¹ in formula I is hydrogen.

7. The method according to claim 1 in which R² in formula I is hydrogen, methyl, ethyl, CF₃, nitro, cyano or halogen.

8. The method according to claim 1 in which R³ in formula I is hydrogen, methyl, ethyl, CF₃, nitro, cyano or halogen.

9. The method according to claim 1 in which n in formula I is 0 or 1.

10. The method according to claim 1 in which Ar in formula I is phenyl, a six-membered heteroaromatic having 1 or 2 nitrogen atoms or a five-membered heteroaromatic having a nitrogen atom and optionally a further heteroatom selected from the group consisting of O, S and N.

11. The method according to claim 10 in which Ar in formula I is phenyl, pyridinyl, pyrimidinyl, pyrazolyl, imidazolyl, oxazolyl, isoxazolyl, thiazolyl, isothiazolyl, furyl, thienyl, or pyrrolyl.

12. The method according to claim 11 in which Ar in formula I is phenyl.

13. The method according to claim 1 in which Y in formula I is oxygen.

14. The use of (hetero)cyclylcarboxamides of the formula I according to claim 1 and of agriculturally useful salts thereof for controlling harmful fungi.

15. A (hetero)cyclylcarboxamide of the formula I 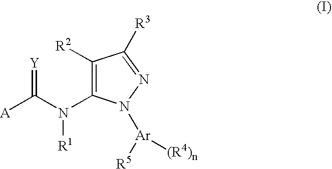
in which the variables n, Y, Ar, R¹, R², R³, R⁴ and R⁵ are as defined in claim 1:
R³ is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy,
and in which
A is an at least monounsaturated 5- or 6-membered heterocycle having 1, 2 or 3 heteroatoms selected from the group consisting of N, O, S, S(═O) and S(═O)₂ as ring members, which heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
or an agriculturally useful salt thereof,
except for (hetero)cyclylcarboxamides of the formula I in which A is 1-phenylpyrazol-5-yl, 1-phenyl-3-methylpyrazol-5-yl, 1,3-dimethyl-4-chloropyrazol-5-yl, 5-nitropyrazol-3-yl, 1-ethyl-3-methyl-4-nitropyrazol-5-yl, 5-methyl-4-nitropyrazol-3-yl, 1-(4-chlorophenyl)-5-trifluoromethylpyrazol-4-yl, 2-thienyl, 3-methylthiophen-2-yl, 2-furyl, 3-furyl, pyrazin-2-yl, 2,5-dihydropyrrol-2-yl, 2,3-dihydro-5-methyl-1,4-thioxin-6-yl or 5-methylisoxazol-3-yl, and Ar is phenyl,
furthermore except for N-[3-methyl-1-(phthalazinyl)pyrazol-5-yl]nicotinamide and N-[3-methyl-1-(phthalazinyl)pyrazol-5-yl]isonicotinamide.

16. A (hetero)cyclylcarboxamide of the formula I′, 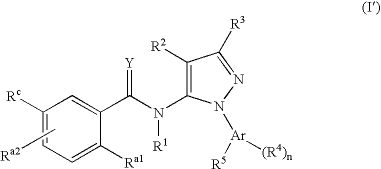
in which the variables n, Y, Ar, R¹, R², R³, R⁴ and R⁵ are as defined in claim 1, with the proviso that R⁵ is different from hydrogen, and in which
R^{a} is C₁-C₄-alkyl, C₁-C₄-alkoxy, C₁-C₄-haloalkyl, C₁-C₄-haloalkoxy or halogen; and
R^{a2} is hydrogen, halogen, nitro, CN, C₁-C₄-alkyl, C₃-C₆-cycloalkyl, C₂-C₄-alkenyl, C₂-C₄-alkynyl or C₁-C₄-alkoxy, where the 5 last-mentioned groups may be substituted by halogen; and
R^{c} is selected from the group consisting of hydrogen, halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy,
or an agriculturally useful salt thereof.

17. A crop protection composition, comprising at least one (hetero)cyclyl-carboxamide of the formula I or I′ according to claim 15 or an agriculturally useful salt thereof.

18. A (hetero)cyclylcarboxamide of the formula I-A 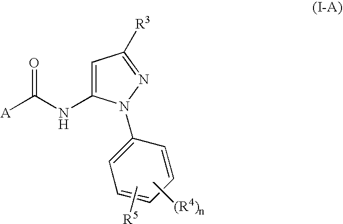
in which
A represents a radical selected from the group consisting of 2-trifluoromethylpyridin-3-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloropyrazol-4-yl, 1-methyl-3-trifluoromethylpyrrol-4-yl, 2-methyl-4-trifluoromethylthiazol-5-yl, 2-methyl-4-difluoromethylthiazol-5-yl, 2-methyl-5-trifluoromethylthiazol-4-yl, 2,5-dimethylthiazol-4-yl, 2-methyl-4-trifluoromethyloxazol-5-yl, 2-trifluoromethylthiophen-3-yl, 5-methyl-2-trifluoromethylthiophen-3-yl, 3-trifluoromethylthiophen-2-yl and 2,5-dimethylfuran-3-yl;
R³ is selected from the group consisting of H, methyl, trifluoromethyl, CN, NO₂ and halogen;
(R⁴)ₙ is either not present or is fluorine, chlorine, bromine, methyl, methoxy, dimethoxy, bromine+chlorine or dichloro; and
R⁵ is hydrogen, halogen, CN, NO₂, NH₂, C(O)NH₂, C(S)NH₂, C₁-C₆-alkyl, C₂-C₆-alkenyl, C₁-C₆-haloalkyl, C₁-C₄-alkoxy, C₁-C₄-haloalkoxy, C₃-C₆-cycloalkyl, C₃-C₆-halocycloalkyl, C₁-C₄-alkylcarbonyl, C₁-C₄-alkoxycarbonyl, C₁-C₄-alkylamino, di-C₁-C₄-alkylamino, C₁-C₄-alkylthio, C₁-C₄-alkylsulfonyl, C₁-C₄-alkylaminocarbonyl, C₁-C₄-dialkylaminocarbonyl, phenyl, phenyl-C₁-C₄-alkyl, where phenyl in the two last-mentioned radicals may be unsubstituted or may carry one, two or three radicals R^{b}, or a group —C(R⁶)═NOR⁷, or R⁴ and R⁵ may also be methylenedioxy, dichloromethylenedioxy or difluoromethylenedioxy, where R⁶, R⁷ and R^{b} are as defined below:
R⁶ is hydrogen, C₁-C₆-alkyl, C₃-C₆-cycloalkyl, C₂-C₆-alkenyl, C₂-C₆-alkynyl, C₁-C₆-halo alkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b};
R⁷ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl and the phenyl group in phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl and phenyl-C₂-C₄-haloalkynyl may be unsubstituted or may carry one, two or three radicals R^{b};
R^{b} is selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
or an agriculturally useful salt thereof.

19. A (hetero)cyclylcarboxamide of the formula I-B 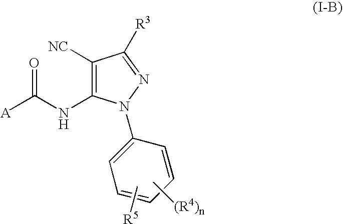
in which the variables n, R³, R⁴ and R⁵ are as defined in claim 1,
A is an at least monounsaturated 5- or 6-membered heterocycle having 1, 2 or 3 heteroatoms selected from the group consisting of N, O, S, S(═O) and S(═O)₂ as ring members, which heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
or an agriculturally useful salt thereof,
except for (hetero)cyclylcarboxamides of the formula I in which A is 1-phenylpyrazol-5-yl, 1-phenyl-3-methylpyrazol-5-yl, 1,3-dimethyl-4-chloropyrazol-5-yl, 5-nitropyrazol-3-yl, 1-ethyl-3-methyl-4-nitropyrazol-5-yl, 5-methyl-4-nitropyrazol-3-yl, 1-(4-chlorophenyl)-5-trifluoromethylpyrazol-4-yl, 2-thienyl, 3-methylthiophen-2-yl, 2-furyl, 3-furyl, pyrazin-2-yl, 2,5-dihydropyrrol-2-yl, 2,3-dihydro-5-methyl-1,4-thioxin-6-yl or 5-methylisoxazol-3-yl.

20. A (hetero)cyclylcarboxamide of the formula I-C, I-D or I-E 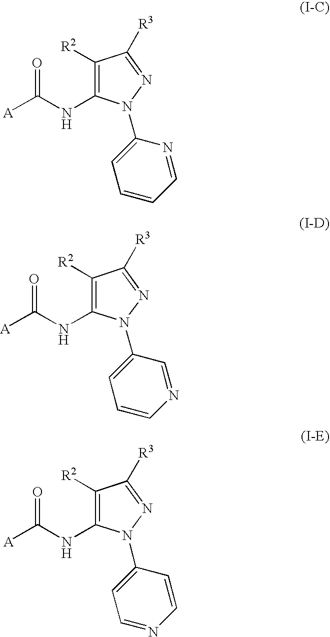
in which
R² is H or CN,
R³ is selected from the group consisting of H, methyl, trifluoromethyl, CN, NO₂ and halogen,
A is a radical A-1a, A-2a or A-3a 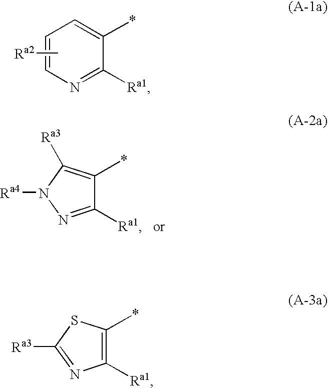
where R^{a1}, R^{a2}, R^{a3} and R^{a4} are as defined below:
R^{a1} is hydrogen, C₁-C₄-alkyl, C₁-C₄-alkoxy, C₁-C₄-haloalkyl, C₁-C₄-haloalkoxy or halogen;
R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen; and
R^{a3} is hydrogen, halogen, nitro, CN, C₁-C₄-alkyl, C₃-C₆-cycloalkyl; and
R^{a4} is hydrogen, C₁-C₄-alkyl or C₁-C₄-haloalkyl;
or an agriculturally useful salt thereof.

21. A (hetero)cyclylcarboxamide of the formula I-F or I-G 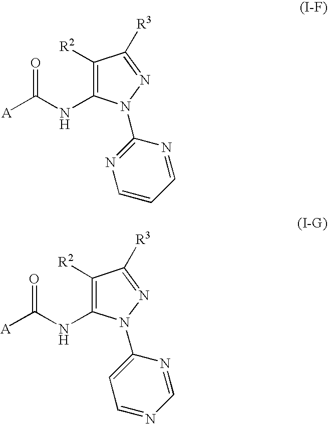
in which the variables n, R² and R³ are as defined in claim 1,
A is an at least monounsaturated 5- or 6-membered heterocycle having 1, 2 or 3 heteroatoms selected from the group consisting of N, O, S, S(═O) and S(═O)₂ as ring members, which heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
or an agriculturally useful salt thereof.
